SEQUENCE LISTING (35 sequences)

SEQ ID NO: 1 - protein, 17 aa, Artificial Sequence
  Description of Artificial Sequence Synthetic peptide
GGGGSGGGGSGGGGSLE

SEQ ID NO: 2 - protein, 20 aa, Artificial Sequence
  Description of Artificial Sequence Synthetic peptide
AAAGGGGSGGGGSGGGGSLE

SEQ ID NO: 3 - protein, 30 aa, Artificial Sequence
  Description of Artificial Sequence Synthetic polypeptide
AAAIEVMYPPPYLGGGGSGGGGSGGGGSLE

SEQ ID NO: 4 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 5 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 6 - protein, 172 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKS

SEQ ID NO: 7 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 8 - protein, 280 aa, Homo sapiens
MAGTWLLLLLALGCPALPTGVGGTPFPSLAPPIMLLVDGKQQMVVVCLVLDVAPPGLDSPIWFSAGNGSALDAFTYGPSPATDGTWTNLAHLSLPSEELASWEPLVCHTGPGAEGHSRSTQPMHLSGEASTARTCPQEPLRGTPGGALWLGVLRLLLFKLLLFDLLLTCSCLCDPAGPLPSPATTTRLRALGSHRLHPATETGGREATSSPRPQPRDRRWGDTPPGRKPGSPVWGEGSYLSSYPTCPAQAWCSRSALRAPSSSLGAFFAGDLPPPLQAGA

SEQ ID NO: 9 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 139 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAAKGAGTASKLTFGTGTRLQVTL

SEQ ID NO: 11 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 133 aa, Homo sapiens
MGTSLLCWMALCLLGADHADTGVSQNPRHNITKRGQNVTFRCDPISEHNRLYWYRQTLGQGPEFLTYFQNEAQLEKSRLLSDRFSAERPKGSFSTLEIQRTEQGDSAMYLCASSLAGLNQPQHFGDGTRLSIL

SEQ ID NO: 13 - protein, 135 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSFSTCSANYGYTFGSGTRLTVV

SEQ ID NO: 14 - DNA, 8791 bp, Homo sapiens
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgatactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagattctagagccgccaccatgcttctcctggtgacaagccttctgctctgtgagttaccacacccagcattcctcctgatcccagacattcagcaggtccagctccagcagtctggccctgaactcgaaaaacctggcgctagcgtgaaaatttcctgtaaagcctccggctactcttttactggctacacaatgaattgggtgaaacagtctcacggcaaatccctcgaatggatcggactcatcacaccctacaatggcgcctcttcctacaaccagaaattccggggcaaggcaacactcactgtggacaaatcatcctctaccgcctacatggatctgctctccctcacatctgaggactccgctgtctacttttgtgcccgaggaggatacgacggacgaggattcgattactggggacagggaacaactgtgaccgtgtctagtggcggcggagggagtggaggcggaggatcttctggcgggggatccgatattgaactcacacagtctcccgctatcatgtctgcttctcccggcgagaaagtgactatgacttgctctgcttcctcttctgtgtcctacatgcactggtaccagcagaaatctggcacatcccctaaacggtggatctacgatactagcaaactggcatccggcgtgcctgggcgattctctggctctggctctggcaactcttactctctcacaatctcatctgtcgaggctgaggacgatgccacatactactgtcagcagtggtctaaacacccactcacattcggcgctggcactaaactggaaataaaagcggccgcaggtggcggcggttctggtggcggcggttctggtggcggcggttctctcgaggatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgataagaattcgatccgcggccgcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 15 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - protein, 111 aa, Artificial Sequence
  Description of Artificial Sequence Synthetic polypeptide
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 17 - protein, 119 aa, Artificial Sequence
  Description of Artificial Sequence Synthetic polypeptide
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 18 - protein, 116 aa, Artificial Sequence
  Description of Artificial Sequence Synthetic polypeptide
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 19 - protein, 10 aa, Artificial Sequence
  Description of Artificial Sequence Synthetic peptide
GGGGSGGGGS

SEQ ID NO: 20 - DNA, 30 bp, Artificial Sequence
  Description of Artificial Sequence Synthetic oligonucleotide
ggtggcggaggttctggaggtggaggttcc

SEQ ID NO: 21 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 22 - protein, 20 aa, Artificial Sequence
  Description of Artificial Sequence Synthetic peptide
  MISC_FEATURE (1)..(20): This sequence may encompass 1-4 “Gly Gly Gly Gly Ser” repeating units
GGGGSGGGGSGGGGSGGGGS

SEQ ID NO: 23 - protein, 20 aa, Artificial Sequence
  Description of Artificial Sequence Synthetic peptide
  MISC_FEATURE (1)..(20): This sequence may encompass 2-4 “Gly Gly Gly Gly Ser” repeating units
GGGGSGGGGSGGGGSGGGGS

SEQ ID NO: 24 - protein, 15 aa, Artificial Sequence
  Description of Artificial Sequence Synthetic peptide
  MISC_FEATURE (1)..(15): This sequence may encompass 1-3 “Gly Gly Gly Gly Ser” repeating units
GGGGSGGGGSGGGGS

SEQ ID NO: 25 - protein, 4 aa, Artificial Sequence
  Description of Artificial Sequence Synthetic peptide
  See specification as filed for detailed description of substitutions and preferred embodiments
GGGS

SEQ ID NO: 26 - protein, 20 aa, Artificial Sequence
  Description of Artificial Sequence Synthetic peptide
GGGGSGGGGSGGGGSGGGGS

SEQ ID NO: 27 - protein, 15 aa, Artificial Sequence
  Description of Artificial Sequence Synthetic peptide
GGGGSGGGGSGGGGS

SEQ ID NO: 28 - protein, 4 aa, Artificial Sequence
  Description of Artificial Sequence Synthetic peptide
GGGS

SEQ ID NO: 29 - RNA, 5000 nt, Artificial Sequence
  Description of Artificial Sequence Synthetic polynucleotide
  misc_feature (1)..(5000): This sequence may encompass 50-5000 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - protein, 30 aa, Artificial Sequence
  Description of Artificial Sequence Synthetic polypeptide
  MISC_FEATURE (1)..(30): This sequence may encompass 1-6 “Gly Gly Gly Gly Ser” repeating units
  See specification as filed for detailed description of substitutions and preferred embodiments
GGGGSGGGGSGGGGSGGGGSGGGGSGGGGS

SEQ ID NO: 31 - RNA, 2000 nt, Artificial Sequence
  Description of Artificial Sequence Synthetic polynucleotide
  misc_feature (1)..(2000): This sequence may encompass 50-2000 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - DNA, 100 bp, Artificial Sequence
  Description of Artificial Sequence Synthetic polynucleotide
tttttttttttttttttttttttttttttttttttttttttttttttttttttttttttttttttttttttttttttttttttttttttttttttttttt

SEQ ID NO: 33 - DNA, 5000 bp, Artificial Sequence
  Description of Artificial Sequence Synthetic polynucleotide
  misc_feature (1)..(5000): This sequence may encompass 50-5000 nucleotides
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 34 - RNA, 5000 nt, Artificial Sequence; identical to SEQ ID NO: 29
  Description of Artificial Sequence Synthetic polynucleotide
  misc_feature (1)..(5000): This sequence may encompass 100-5000 nucleotides

SEQ ID NO: 35 - RNA, 400 nt, Artificial Sequence
  Description of Artificial Sequence Synthetic polynucleotide
  misc_feature (1)..(400): This sequence may encompass 100-400 nucleotides
  See specification as filed for detailed description of substitutions and preferred embodiments
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2022/0347214 A1
(43) Pub. Date: Nov. 3, 2022

(54) COMPOSITIONS AND METHODS FOR TCR REPROGRAMMING USING FUSION PROTEINS

(71) Applicant: TCR2 THERAPEUTICS INC., Cambridge, MA (US)

(72) Inventor: Alfonso QUINTAS-CARDAMA, Newton, MA (US)

(21) Appl. No.: 17/441,606

(22) PCT Filed: Mar. 20, 2020
(86) PCT No.: PCT/US2020/023928

§ 371 Date: Sep. 21, 2021

Related U.S. Application Data
(60) Provisional application No. 62/822,683, filed on Mar. 22, 2019.

(51) Int. Cl. A61K 35/17; A61K 31/7076; A61K 31/664; A61K 39/395; A61P 35/00

(52) U.S. Cl. CPC A61K 35/17; A61K 31/7076; A61K 31/664; A61K 39/3955; A61P 35/00

(57) ABSTRACT

Disclosed herein are dosage and administration methods for treatment of a mesothelin-expressing cancer in a human comprising administration of, e.g., a plurality of anti-mesothelin T cell receptor fusion protein-expressing T cells.

### CROSS-REFERENCE

This application claims the benefit of U.S. Provisional Application No. 62/822,683, filed on Mar. 22, 2019, which is incorporated herein by reference in its entirety.

### BACKGROUND

Most patients with late-stage solid tumors are incurable with standard therapy. In addition, traditional treatment options often have serious side effects. Numerous attempts have been made to engage a patient's immune system for rejecting cancerous cells, an approach collectively referred to as cancer immunotherapy. However, several obstacles make it rather difficult to achieve clinical effectiveness. Although hundreds of so-called tumor antigens have been identified, these are often derived from self and thus can direct the cancer immunotherapy against healthy tissue, or are poorly immunogenic. Furthermore, cancer cells use multiple mechanisms to render themselves invisible or hostile to the initiation and propagation of an immune attack by cancer immunotherapies.

Recent developments using chimeric antigen receptor (CAR) modified autologous T cell therapy, which relies on redirecting genetically engineered T cells to a suitable cell-surface molecule on cancer cells, show promising results in harnessing the power of the immune system to treat B cell malignancies (see, e.g., Sadelain et al., Cancer Discovery 3:388-398 (2013)). The clinical results with CD-19-specific CAR T cells (called CTL019) have shown complete remissions in patients suffering from chronic lymphocytic leukemia (CLL) as well as in childhood acute lymphoblastic leukemia (ALL) (see, e.g., Kalos et al., Sci Transl Med 3:95ra73 (2011), Porter et al., NEJM 365:725-733 (2011), Grupp et al., NEJM 368:1509-1518 (2013)). An alternative approach is the use of T cell receptor (TCR) alpha and beta chains selected for a tumor-associated peptide antigen for genetically engineering autologous T cells. These TCR chains will form complete TCR complexes and provide the T cells with a TCR for a second defined specificity. Encouraging results were obtained with engineered autologous T cells expressing NY-ESO-1-specific TCR alpha and beta chains in patients with synovial carcinoma.

Besides the ability of genetically modified T cells expressing a CAR or a second TCR to recognize and destroy respective target cells in vitro/ex vivo, successful patient therapy with engineered T cells requires the T cells to be capable of strong activation, expansion, persistence over time, and, in case of relapsing disease, to enable a ‘memory’ response. High and manageable clinical efficacy of CAR T cells is currently limited to BCMA- and CD-19-positive B cell malignancies and to NY-ESO-1-peptide expressing synovial sarcoma patients expressing HLA-A2. There is a clear need to improve genetically engineered T cells to more broadly act against various human malignancies. Described herein are novel fusion proteins of TCR subunits, including CD3 epsilon, CD3gamma and CD3 delta, and of TCR alpha and TCR beta chains with binding domains specific for cell surface antigens that have the potential to overcome limitations of existing approaches.

### SUMMARY

Disclosed herein are compositions and methods for the treatment of a human having a cancer, e.g., a cancer comprising cells that express mesothelin. In one aspect is a method for the treatment of a human patient diagnosed with a mesothelin (MSLN)-expressing unresectable, metastatic, or recurrent cancer, the method comprising administering to the patient a first dose comprising an amount of transduced anti-MSLN T cell receptor fusion protein (TFP) T cells, and further comprising administering one or more additional doses, wherein the first dose and each additional doses comprise from about 5×10⁷ to about 1×10⁹ transduced cells/m2. In one embodiment, the cancer comprises malignant pleural mesothelioma (MPM), non-small cell lung cancer (NSCLC), serous ovarian adenocarcinoma, or cholangiocarcinoma.

In one embodiment, the method comprises administering one, two, three, or more than three additional doses of anti-MSLN TFP T cells in evenly spaced increments. In another embodiment, the method comprises administering four doses of anti-MSLN TFP T cells in evenly spaced increments, comprising a first dose, a second dose, a third dose, and a fourth dose. In one embodiment, the anti-MSLN TFP T cells are administered via intravenous infusion.

In another embodiment, the anti-MSLN TFP T cells are administered as a single agent. In one embodiment, the dose of anti-MSLN TFP T cells is about 5×10⁷/m². In another embodiment, the dose of anti-MSLN TFP T cells is about 1×10⁸/m². In another embodiment, the dose of anti-MSLN TFP T cells is about 5×10⁸/m². In another embodiment, the dose of anti-MSLN TFP T cells is about 1×10⁹/m². In another embodiment, a dose range of ±15% of the target dose may be administered. the second dose of anti-MSLN TFP T cells is administered no sooner than 60 days and no later than 12 months following administration of the first dose of anti-MSLN TFP T cells.

In one embodiment, the method further comprises the step of administering to the patient a lymphodepleting chemotherapy regimen prior to administration of the first dose of anti-MSLN TFP T cells. In another embodiment, the lymphodepleting chemotherapy regimen comprises administration of four doses of fludarabine and three doses of cyclophosphamide. In another embodiment, the lymphodepleting chemotherapy comprises fludarabine administered to the patient at a level of 30 mg/m²/day on days −7 through −4 relative to administration of anti-MSLN TFP T cells, and further comprises cyclophosphamide administered at a level of 600 mg/m²/day on days −6 through −4 relative to administration of anti-MSLN TFP T cells.

In one embodiment, the method further comprises co-administration of a chemotherapeutic agent. In one embodiment, the chemotherapeutic agent is administered four times at three dose levels, comprising a first dose, a second dose, a third dose, and a fourth dose. In another embodiment, the first dose of the chemotherapeutic agent is administered three weeks after administration of anti-MSLN TFP T cells, and wherein subsequent doses are administered every three weeks thereafter. In one embodiment, the chemotherapeutic agent is administered every three weeks. In another embodiment the chemotherapeutic agent comprises chemotherapy. In another embodiment, chemotherapeutic agent comprises pembrolizumab.

In another aspect is provided a method for the treatment of a human patient diagnosed with a mesothelin (MSLN)-expressing unresectable, metastatic, or recurrent cancer, the method comprising the steps of administering to the patient a lymphodepleting chemotherapy regimen; administering to the patient a plurality of doses, each dose comprising an amount of transduced anti-MSLN TFP T cells, at an interval less than about sixty days between doses; and optionally administering to the patient an effective amount of a chemotherapeutic agent.

In one embodiment, the cancer comprises malignant pleural mesothelioma (MPM), non-small cell lung cancer (NSCLC), serous ovarian adenocarcinoma, or cholangiocarcinoma. In another embodiment, the method comprises administering four doses of anti-MSLN TFP T cells in evenly spaced increments, comprising a first dose, a second dose, a third dose, and a fourth dose. In one embodiment, each dose comprises from about 5×10⁷ to about 1×10⁹ transduced cells/m². In one embodiment, each dose is about 5×10⁷/m². In another embodiment, each dose about 1×10⁸/m². In another embodiment, each dose is about 5×10⁸/m². In another embodiment, each dose is about 1×10⁹/m².

In one embodiment, each dose comprises a dose range of ±15% of the target dose may be administered.

In one embodiment, the second dose of anti-MSLN TFP T cells is administered no sooner than 60 days and no later than 12 months following administration of the first dose of anti-MSLN TFP T cells. In one embodiment, the dose is administered via intravenous infusion. In another embodiment, the method further comprises the step of administering to the patient a lymphodepleting chemotherapy regimen prior to administration of the first dose of anti-MSLN TFP T cells. In another embodiment, the lymphodepleting chemotherapy regimen comprises administration of four doses of fludarabine and three doses of cyclophosphamide. In another embodiment, the lymphodepleting chemotherapy comprises fludarabine administered to the patient at a level of 30 mg/m²/day on days −7 through −4 relative to administration of anti-MSLN TFP T cells, and further comprises cyclophosphamide administered at a level of 600 mg/m²/day on days −6 through −4 relative to administration of anti-MSLN TFP T cells.

In one embodiment, the method further comprises administering a chemotherapeutic agent, wherein the chemotherapeutic agent is administered four times at three dose levels, comprising a first dose, a second dose, a third dose, and a fourth dose. In one embodiment, the first dose of the chemotherapeutic agent is administered three weeks after administration of anti-MSLN TFP T cells, and wherein subsequent doses are administered every three weeks thereafter. In one embodiment, the chemotherapeutic agent is administered every three weeks. In one embodiment, the chemotherapeutic agent comprises chemotherapy. In one embodiment, the chemotherapeutic agent comprises pembrolizumab.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph showing the cytolytic response of anti-MSLN TFP T cells to mesothelin-positive MSTO-MSLN-LUC tumor cells at a 1:10 effector to target cell ratio.

FIGS. 2A-D are four graphs depicting that anti-MSLN TFP T cells secretes IFNγ and IL-2 in response to mesothelin-positive MSTO-MSLN-LUC tumor cells at 10 to 1 effector to tumor cell ratio.

FIG. 3 shows the evaluation of anti-tumor activity of anti-MSLN TFP T cells in mesothelioma tumor model, as described in Example 3. Each line represents the mean tumor volume for each group and the error bars represent the standard deviation.

FIGS. 4A-B show the mediation of rapid tumor growth by anti-MSLN TFP T cells both in a primary and relapsing model of mesothelioma. Each line indicates the mean and standard deviation for each group.

FIG. 5A is a schematic diagram of in vivo study for anti-MSLN TFP T cell expansion, differentiation and activation.

FIG. 5B is a graph showing tumor volumes measured on day 6 after T cell injection.

FIG. 5C is a graph showing the level of soluble MSLN (sMSLN) in plasma on day 7 after T cell injection.

FIG. 5D is tissue section images showing immunohistochemistry analysis of tumor samples harvested on day 7 for tumor burden (anti-MSLN staining, light brown) and T cell infiltration into tumor (anti-human CD3, dark purple). Images shown here are representative of 4 (tumor only group), 6 (non-transduced (NT) T cell group), or 9 mice (anti-MSLN TFP T cell group). Each symbol in the plots (A, B and C) represents an individual animal in the study. Lines indicate the average of the results of all animals in the treatment group, error bars represent the standard error of the mean (SEM). **, p<0.01, student t-test.

FIGS. 6A-B show the evaluation of anti-tumor activity of anti-MSLN TFP T cells in lung cancer tumor model. FIG. 6A is a schematic diagram of in vivo study for anti-MSLN TFP T cells testing anti-tumor efficacy, expansion and activation. FIG. 6B shows tumor volumes measurement for PBS (n=5), NT T cell (n=14) and anti-MSLN TFP T cells (n=14). Data shown are the mean of the tumor volumes for each group at each time point. Error bar indicates the standard deviation of the of the measurement for each group at each time point.

FIGS. 7A-B show growth of ovarian cancer OVCAR3-luc in NSG mice treated with T cells as shown by bioluminescent imaging of mice. FIG. 7A is a schematic depicting the overall study design. FIG. 7B is a graph showing tumor burden assessed by in vivo bioluminescent imaging following IP inoculation with OVCAR3-LUC ovarian carcinoma cells and i.v. administration of vehicle (downward facing black triangles; n=7), NT T cells (upward facing black triangles; n=7), and anti-MSLN TFP T cells (gray circles; n=7). CD=cluster of differentiation; d=study day; IP=intraperitoneal; i.v.=intravenous; NSG=non-obese diabetic.

FIG. 7C is two graphs showing circulating levels of human (CD3-positive; left panel) and TFP-positive (right panel) cells in blood following administration of PBS (black circles; n=7), NT T cells (black squares; n=7), and anti-MSLN TFP T cells (black triangles; n=7). CD=cluster of differentiation; d=study day; IP=intraperitoneal; i.v.=intravenous; NSG=non-obese diabetic severe combined immunodeficient gamma; NTD=nontransduced; PBS=phosphate-buffered saline; PD=pharmacodynamics; TFP=T cell receptor fusion construct.

### DETAILED DESCRIPTION

Described herein are methods of adoptive cell therapy for treating a cancer, e.g., a mesothelin-expressing cancer, using TFP molecules direct to mesothelin-expressing tumor cells.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 4th ed. (2012) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

### Adoptive T Cell Therapy

Adoptive T cell therapy (ACT) is a therapeutic modality that involves the manipulation of a cancer patient's own T cells to endow them with anti-tumor activity. This is accomplished through the collection, ex vivo activation, modification and expansion, and re-infusion into the patient. The objective of the process is the generation of potent and cancer antigen-specific T cell immunity. Tumor-associated antigens can be classified into 3 major groups:
1. Antigens present in healthy tissue but over-expressed in tumors, usually because they confer a growth advantage to the cancer cell.
2. Neo-antigens arising from somatic mutations in cancer cells.
3. Cancer germline antigens, which are proteins expressed on germline cells, which reside in immunoprivileged sites, and therefore are not vulnerable to autoimmune T cell targeting.

The first successful application of ACT was the use of tumor infiltrating lymphocytes (TILs), which rendered clinical responses in approximately 50% of patient with malignant melanoma (Topalian et. al., 1988). The wide applicability of this therapeutic modality was hindered by the requisite surgery to procure tissue from which to isolate TILs, the difficulties in successfully isolating and expanding TILs, and the difficulty in reproducing similar results in other malignancies. Gene-transfer-based strategies were developed to overcome the immune tolerance on the tumor-specific T cell repertoire. These approaches redirect T cells to effectively target tumor antigens through the transfer of affinity-optimized T cell receptors (TCRs) or synthetic chimeric antigen receptors (CARs) via retrovirus- or lentivirus-based stable transduction. The CAR T cells represent the most extensively characterized ACT platform. CAR T cells are autologous T cells that have been re-programmed to target surface-expressed cancer associated antigens, typically through the inclusion of a single chain antibody variable fragment (scFv). These binding domains are fused to co-stimulatory domains as well as the CD3 chain and subsequently transfected into autologous T cells using viral or non-viral transduction processes. Upon binding to its cognate antigen, CAR T phosphorylates the immunoreceptor tyrosine-based activation motifs (ITAMs) within the CD3 zeta chain. This serves as the initiating T cell activation signal and is critical for CAR T mediated lysis of tumor antigens. Concurrently, scFv binding also stimulates the fused co-simulation domains (usually CD28 or 4-1BB) which provide important expansion and survival signals. Two CD19-directed CAR T cell approaches were approved in 2017 by FDA for the treatment of patients with either pediatric acute lymphoblastic leukemia (ALL) or diffuse large B-cell lymphoma (DLBCL), respectively: tisagenlecleucel (Kymriah™) and axicabtagene cileucel (Yescarta™) (CBER, 2017a; CBER 2017b). The former was also approved by FDA in 2018 for the treatment of patients with relapsed/refractory DLBCL. Notwithstanding this activity in hematological malignancies, CAR T cells have failed to induce significant clinical efficacy against solid cancers, largely due to T cell exhaustion and very limited persistence. By utilizing only 1 (CD3ζ chain) of the 6 distinct T cell receptor subunits in combination with a costimulatory domain, CARs operate outside of the natural TCR signaling complex. The failure to initiate and harness a complete TCR response is arguably a primary underlying factor preventing CAR T cell success in solid tumor indications.

### TFP Technology

In some embodiments, the isolated TFP molecules comprise a TCR extracellular domain that comprises an extracellular domain or portion thereof of a protein selected from the group consisting of the alpha or beta chain of the T cell receptor, CD3 delta, CD3 epsilon, or CD3 gamma, or an amino acid sequence having at least one, two or three modifications but not more than 20, 10 or 5 modifications thereto. In some embodiments, the anti-mesothelin binding domain is connected to the TCR extracellular domain by a linker sequence. In some instances, the linker region comprises (G4S)n, wherein n=1 to 4. In some instances, the linker sequence comprises a long linker (LL) sequence. In some instances, the long linker sequence comprises (G4S)n, wherein n=2 to 4. In some instances, the linker sequence comprises a short linker (SL) sequence. In some instances, the short linker sequence comprises (G4S)n, wherein n=1 to 3.

In some embodiments, the isolated TFP molecules further comprise a sequence encoding a costimulatory domain. In other embodiments, the isolated TFP molecules further comprise a sequence encoding an intracellular signaling domain. In yet other embodiments, the isolated TFP molecules further comprise a leader sequence.

Also provided herein are vectors that comprise a nucleic acid molecule encoding any of the previously described TFP molecules. In some embodiments, the vector is selected from the group consisting of a DNA, a RNA, a plasmid, a lentivirus vector, adenoviral vector, or a retrovirus vector. In some embodiments, the vector further comprises a promoter. In some embodiments, the vector is an in vitro transcribed vector. In some embodiments, a nucleic acid sequence in the vector further comprises a poly(A) tail. In some embodiments, a nucleic acid sequence in the vector further comprises a 3′UTR.

Also provided herein are cells that comprise any of the described vectors. In some embodiments, the cell is a human T cell. In some embodiments, the cell is a CD8+ or CD4+ T cell. In other embodiments, the cell is a CD8+CD4+ T cell. In other embodiments, the cell is a naive T-cell, memory stem T cell, central memory T cell, double negative T cell, effector memory T cell, effector T cell, ThO cell, TcO cell, Th1 cell, Tc1 cell, Th2 cell, Tc2 cell, Th17 cell, Th22 cell, gamma/delta T cell, alpha/beta T cell, natural killer (NK) cell, natural killer T (NKT) cell, hematopoietic stem cell and pluripotent stem cell. In other embodiments, the cells further comprise a nucleic acid encoding an inhibitory molecule that comprises a first polypeptide that comprises at least a portion of an inhibitory molecule, associated with a second polypeptide that comprises a positive signal from an intracellular signaling domain. In some instances, the inhibitory molecule comprise first polypeptide that comprises at least a portion of PD1 and a second polypeptide comprising a costimulatory domain and primary signaling domain.

In another aspect, provided herein are isolated TFP molecules that comprise a human or humanized anti-mesothelin binding domain, a TCR extracellular domain, a transmembrane domain, and an intracellular signaling domain, wherein the TFP molecule is capable of functionally interacting with an endogenous TCR complex and/or at least one endogenous TCR polypeptide.

In another aspect, provided herein are isolated TFP molecules that comprise a human or humanized anti-mesothelin binding domain, a TCR extracellular domain, a transmembrane domain, and an intracellular signaling domain, wherein the TFP molecule is capable of functionally integrating into an endogenous TCR complex.

In another aspect, provided herein are human CD8+ or CD4+ T cells that comprise at least two TFP molecules, the TFP molecules comprising a human or humanized anti-mesothelin binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein are protein complexes that comprise i) a TFP molecule comprising a human or humanized anti-mesothelin binding domain, a TCR extracellular domain, a transmembrane domain, and an intracellular domain; and ii) at least one endogenous TCR complex.

In some embodiments, the TCR comprises an extracellular domain or portion thereof of a protein selected from the group consisting of the alpha or beta chain of the T cell receptor, CD3 delta, CD3 epsilon, or CD3 gamma. In some embodiments, the anti-mesothelin binding domain is connected to the TCR extracellular domain by a linker sequence. In some instances, the linker region comprises (G4S)n, wherein n=1 to 4. In some instances, the linker sequence comprises a long linker (LL) sequence. In some instances, the long linker sequence comprises (G4S)n, wherein n=2 to 4. In some instances, the linker sequence comprises a short linker (SL) sequence. In some instances, the short linker sequence comprises (G4S)n, wherein n=1 to 3.

Also provided herein are human CD8+ or CD4+ T cells that comprise at least two different TFP proteins per any of the described protein complexes.

In another aspect, provided herein is a population of human CD8+ or CD4+ T cells, wherein the T cells of the population individually or collectively comprise at least two TFP molecules, the TFP molecules comprising a human or humanized anti-mesothelin binding domain, a TCR extracellular domain, a transmembrane domain, and an intracellular domain, wherein the TFP molecule is capable of functionally interacting with an endogenous TCR complex and/or at least one endogenous TCR polypeptide in, at and/or on the surface of the human CD8+ or CD4+ T cell.

In another aspect, provided herein is a population of human CD8+ or CD4+ T cells, wherein the T cells of the population individually or collectively comprise at least two TFP molecules encoded by an isolated nucleic acid molecule provided herein.

In another aspect, provided herein are methods of making a cell comprising transducing a T cell with any of the described vectors.

In another aspect, provided herein are methods of generating a population of RNA-engineered cells that comprise introducing an in vitro transcribed RNA or synthetic RNA into a cell, where the RNA comprises a nucleic acid encoding any of the described TFP molecules.

In another aspect, provided herein are methods of providing an anti-tumor immunity in a mammal that comprise administering to the mammal an effective amount of a cell expressing any of the described TFP molecules. In some embodiments, the cell is an autologous T cell. In some embodiments, the cell is an allogeneic T cell. In some embodiments, the mammal is a human.

In another aspect, provided herein are methods of treating a mammal having a disease associated with expression of mesothelin that comprise administering to the mammal an effective amount of the cell of comprising any of the described TFP molecules. In some embodiments, the disease associated with mesothelin expression is selected from a proliferative disease such as a cancer or malignancy or a precancerous condition such as a pancreatic cancer, an ovarian cancer, a stomach cancer, a lung cancer, or an endometrial cancer, or is a non-cancer related indication associated with expression of mesothelin.

In some embodiments, the cells expressing any of the described TFP molecules are administered in combination with an agent that ameliorates one or more side effects associated with administration of a cell expressing a TFP molecule. In some embodiments, the cells expressing any of the described TFP molecules are administered in combination with an agent that treats the disease associated with mesothelin.

Also provided herein are any of the described isolated nucleic acid molecules, any of the described isolated polypeptide molecules, any of the described isolated TFPs, any of the described protein complexes, any of the described vectors or any of the described cells for use as a medicament.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

As used herein, “about” can mean plus or minus less than 1 or 1, 2, 3, 4, 5, 6, 7, 8, 9, 10, 11, 12, 13, 14, 15, 16, 17, 18, 19, 20, 25, 30, or greater than 30 percent, depending upon the situation and known or knowable by one skilled in the art.

As used herein the specification, “subject” or “subjects” or “individuals” may include, but are not limited to, mammals such as humans or non-human mammals, e.g., domesticated, agricultural or wild, animals, as well as birds, and aquatic animals. “Patients” are subjects suffering from or at risk of developing a disease, disorder or condition or otherwise in need of the compositions and methods provided herein.

As used herein, “treating” or “treatment” refers to any indicia of success in the treatment or amelioration of the disease or condition. Treating can include, for example, reducing, delaying or alleviating the severity of one or more symptoms of the disease or condition, or it can include reducing the frequency with which symptoms of a disease, defect, disorder, or adverse condition, and the like, are experienced by a patient. As used herein, “treat or prevent” is sometimes used herein to refer to a method that results in some level of treatment or amelioration of the disease or condition, and contemplates a range of results directed to that end, including but not restricted to prevention of the condition entirely.

As used herein, “preventing” refers to the prevention of the disease or condition, e.g., tumor formation, in the patient. For example, if an individual at risk of developing a tumor or other form of cancer is treated with the methods of the present disclosure and does not later develop the tumor or other form of cancer, then the disease has been prevented, at least over a period of time, in that individual.

The term “antigen-binding domain” means the portion of an antibody that is capable of specifically binding to an antigen or epitope. One example of an antigen-binding domain is an antigen-binding domain formed by a VH-VL dimer of an antibody. Another example of an antigen-binding domain is an antigen-binding domain formed by diversification of certain loops from the tenth fibronectin type III domain of an Adnectin.

As used herein, a “therapeutically effective amount” is the amount of a composition or an active component thereof sufficient to provide a beneficial effect or to otherwise reduce a detrimental non-beneficial event to the individual to whom the composition is administered. By “therapeutically effective dose” herein is meant a dose that produces one or more desired or desirable (e.g., beneficial) effects for which it is administered, such administration occurring one or more times over a given period of time. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

As used herein, a “T cell receptor (TCR) fusion protein” or “TFP” includes a recombinant polypeptide derived from the various polypeptides comprising the TCR that is generally capable of i) binding to a surface antigen on target cells and ii) interacting with other polypeptide components of the intact TCR complex, typically when co-located in or on the surface of a T cell. A “TFP T cell” is a T cell that has been transduced (e.g., according to the methods disclosed herein) and that expresses a TFP, e.g., incorporated into the natural TCR. In some embodiments, the T cell is a CD4+ T cell, a CD8+ T cell, or a CD4+/CD8+ T cell. In some embodiments, the TFP T cell is an NK cell. In some embodiments, the TFP T cell is a gamma-delta T cell.

As used herein, the term “mesothelin” also known as MSLN or CAK1 antigen or Pre-pro-megakaryocyte-potentiating factor, refers to the protein that in humans is encoded by the MSLN (or Megakaryocyte-potentiating factor (MPF)) gene. Mesothelin is a 40 kDa protein present on normal mesothelial cells and overexpressed in several human tumors, including mesothelioma and ovarian and pancreatic adenocarcinoma. The mesothelin gene encodes a precursor protein that is processed to yield mesothelin which is attached to the cell membrane by a glycophosphatidylinositol linkage and a 31-kDa shed fragment named megakaryocyte-potentiating factor (MPF). Mesothelin may be involved in cell adhesion, but its biological function is not known. Mesothelin is a tumour differentiation antigen that is normally present on the mesothelial cells lining the pleura, peritoneum and pericardium. Mesothelin is an antigenic determinant detectable on mesothelioma cells, ovarian cancer cells, pancreatic adenocarcinoma cell and some squamous cell carcinomas (see, e.g., Kojima et al., J. Biol. Chem. 270:21984-21990(1995) and Onda et al., Clin. Cancer Res. 12:4225-4231(2006)). Mesothelin interacts with CA125/MUC16 (see, e.g., Rump et al., J. Biol. Chem. 279:9190-9198(2004) and Ma et al., J. Biol. Chem. 287:33123-33131(2012)).

The human and murine amino acid and nucleic acid sequences can be found in a public database, such as GenBank, UniProt and Swiss-Prot. For example, the amino acid sequence of human mesothelin can be found as UniProt/Swiss-Prot Accession No. Q13421. The human mesothelin polypeptide canonical sequence is UniProt Accession No. Q13421 (or Q13421-1):

(SEQ ID NO: 15)
MALPTARPLLGSCGTPALGSLLFLLFSLGWVQPSRTLAGETGQEAAPLDG
VLANPPNISSLSPRQLLGFPCAEVSGLSTERVRELAVALAQKNVKLSTEQ
LRCLAHRLSEPPEDLDALPLDLLLFLNPDAFSGPQACTRFFSRITKANVD
LLPRGAPERQRLLPAALACWGVRGSLLSEADVRALGGLACDLPGRFVAES
AEVLLPRLVSCPGPLDQDQQEAARAALQGGGPPYGPPSTWSVSTMDALRG
LLPVLGQPIIRSIPQGIVAAWRQRSSRDPSWRQPERTILRPRFRREVEKT
ACPSGKKAREIDESLIFYKKWELEACVDAALLATQMDRVNAIPFTYEQLD
VLKHKLDELYPQGYPESVIQHLGYLFLKMSPEDIRKWNVTSLETLKALLE
VNKGHEMSPQAPRRPLPQVATLIDRFVKGRGQLDKDTLDTLTAFYPGYLC
SLSPEELSSVPPSSIWAVRPQDLDTCDPRQLDVLYPKARLAFQNMNGSEY
FVKIQSFLGGAPTEDLKALSQQNVSMDLATFMKLRTDAVLPLTVAEVQKL
LGPHVEGLKAEERHRPVRDWILRQRQDDLDTLGLGLQGGIPNGYLVLDLS
MQEALSGTPCLLGPGPVLTVLALLLASTLA.

The nucleotide sequence encoding human mesothelin transcript variant 1 can be found at Accession No. NM005823. The nucleotide sequence encoding human mesothelin transcript variant 2 can be found at Accession No. NM013404. The nucleotide sequence encoding human mesothelin transcript variant 3 can be found at Accession No. NM001177355. Mesothelin is expressed on mesothelioma cells, ovarian cancer cells, pancreatic adenocarcinoma cell and squamous cell carcinomas (see, e.g., Kojima et al., J. Biol. Chem. 270:21984-21990(1995) and Onda et al., Clin. Cancer Res. 12:4225-4231(2006)). Other cells that express mesothelin are provided below in the definition of “disease associated with expression of mesothelin.” Mesothelin also interacts with CA125/MUC16 (see, e.g., Rump et al., J. Biol. Chem. 279:9190-9198(2004) and Ma et al., J. Biol. Chem. 287:33123-33131(2012)). In one example, the antigen-binding portion of TFPs recognizes and binds an epitope within the extracellular domain of the mesothelin protein as expressed on a normal or malignant mesothelioma cell, ovarian cancer cell, pancreatic adenocarcinoma cell, or squamous cell carcinoma cell.

The term “antibody,” as used herein, refers to a protein, or polypeptide sequences derived from an immunoglobulin molecule, which specifically binds to an antigen. Antibodies can be intact immunoglobulins of polyclonal or monoclonal origin, or fragments thereof and can be derived from natural or from recombinant sources.

The terms “antibody fragment” or “antibody binding domain” refer to at least one portion of an antibody, or recombinant variants thereof, that contains the antigen binding domain, i.e., an antigenic determining variable region of an intact antibody, that is sufficient to confer recognition and specific binding of the antibody fragment to a target, such as an antigen and its defined epitope. Examples of antibody fragments include, but are not limited to, Fab, Fab′, F(ab′)₂, and Fv fragments, single-chain (sc)Fv (“scFv”) antibody fragments, linear antibodies, single domain antibodies (abbreviated “sdAb”) (either V_{L} or V_{H}), camelid V_{HH} domains, and multi-specific antibodies formed from antibody fragments.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single polypeptide chain, and wherein the scFv retains the specificity of the intact antibody from which it is derived.

“Heavy chain variable region” or “VH” (or, in the case of single domain antibodies, e.g., nanobodies, “VHH”) with regard to an antibody refers to the fragment of the heavy chain that contains three CDRs interposed between flanking stretches known as framework regions, these framework regions are generally more highly conserved than the CDRs and form a scaffold to support the CDRs.

Unless specified, as used herein a scFv may have the VL and VH variable regions in either order, e.g., with respect to the N-terminal and C-terminal ends of the polypeptide, the scFv may comprise VL-linker-VH or may comprise VH-linker-VL.

The portion of the TFP composition of the disclosure comprising an antibody or antibody fragment thereof may exist in a variety of forms where the antigen binding domain is expressed as part of a contiguous polypeptide chain including, for example, a single domain antibody fragment (sdAb) or heavy chain antibodies HCAb, a single chain antibody (scFv) derived from a murine, humanized or human antibody (Harlow et al., 1999, In: Using Antibodies: A Laboratory Manual, Cold Spring Harbor Laboratory Press, N.Y.; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a TFP composition of the disclosure comprises an antibody fragment. In a further aspect, the TFP comprises an antibody fragment that comprises a scFv or a sdAb.

The term “antigen” or “Ag” refers to a molecule that is capable of being bound specifically by an antibody, or otherwise provokes an immune response. This immune response may involve either antibody production, or the activation of specific immunologically-competent cells, or both.

The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the disclosure in prevention of the occurrence of tumor in the first place.

The term “autologous” refers to any material derived from the same individual to whom it is later to be re-introduced into the individual.

The term “allogeneic” refers to any material derived from a different animal of the same species or different patient as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lung cancer, and the like.

The phrase “disease associated with expression of mesothelin” includes, but is not limited to, a disease associated with expression of mesothelin or condition associated with cells which express mesothelin including, e.g., proliferative diseases such as a cancer or malignancy or a precancerous condition In one aspect, the cancer is a mesothelioma. In one aspect, the cancer is a pancreatic cancer. In one aspect, the cancer is an ovarian cancer. In one aspect, the cancer is a stomach cancer. In one aspect, the cancer is a lung cancer. In one aspect, the cancer is an endometrial cancer. Non-cancer related indications associated with expression of mesothelin include, but are not limited to, e.g., autoimmune disease, (e.g., lupus, rheumatoid arthritis, colitis), inflammatory disorders (allergy and asthma), and transplantation.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TFP of the disclosure can be replaced with other amino acid residues from the same side chain family and the altered TFP can be tested using the functional assays described herein.

The term “stimulation” refers to a primary response induced by binding of a stimulatory domain or stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, and/or reorganization of cytoskeletal structures, and the like.

The term “stimulatory molecule” or “stimulatory domain” refers to a molecule or portion thereof expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or “ITAM”. Examples of an ITAM containing primary cytoplasmic signaling sequence that is of particular use in the disclosure includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”) and CD66d.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T cells may recognize these complexes using their T cell receptors (TCRs). APCs process antigens and present them to T cells.

An “intracellular signaling domain,” as the term is used herein, refers to an intracellular portion of a molecule. The intracellular signaling domain generates a signal that promotes an immune effector function of the TFP containing cell, e.g., a TFP-expressing T cell. Examples of immune effector function, e.g., in a TFP-expressing T cell, include cytolytic activity and T helper cell activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation.

A primary intracellular signaling domain can comprise an ITAM (“immunoreceptor tyrosine-based activation motif”). Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, and CD66d DAP10 and DAP12.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class 1 molecule, BTLA and a Toll ligand receptor, as well as DAP10, DAP12, CD30, LIGHT, OX40, CD2, CD27, CD28, CDS, ICAM-1, LFA-1 (CD11a/CD18) and 4-1BB (CD137). A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like. The intracellular signaling domain can comprise the entire intracellular portion, or the entire native intracellular signaling domain, of the molecule from which it is derived, or a functional fragment thereof. The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or equivalent residues from non-human species, e.g., mouse, rodent, monkey, ape and the like.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain one or more introns.

The term “effective amount” or “therapeutically effective amount” are used interchangeably herein, and refer to an amount of a compound, formulation, material, or composition, as described herein effective to achieve a particular biological or therapeutic result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR™ gene delivery technology from Oxford BioMedica, the LENTIMAX™ vector system from Lentigen, and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Human” or “fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the transcription machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “linker” and “flexible polypeptide linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)ₙ, where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄Ser)₄ or (Gly₄Ser)₃. In another embodiment, the linkers include multiple repeats of (Gly₂Ser), (GlySer) or (Gly₃Ser). Also included within the scope of the disclosure are linkers described in WO2012/138475 (incorporated herein by reference). In some instances, the linker sequence comprises (G₄S)ₙ, wherein n=2 to 4. In some instances, the linker sequence comprises (G₄S)ₙ, wherein n=1 to 3.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, which has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000, preferably greater than 64, more preferably greater than 100, most preferably greater than 300 or 400. Poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human). Exemplary subjects include humans, monkeys, dogs, cats, mice, rats, cows, horses, camels, goats, rabbits, and sheep. In certain embodiments, the subject is a human. A “patient” is a subject suffering from or at risk of developing a disease, disorder or condition or otherwise in need of the compositions and methods provided herein.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

In the context of the present disclosure, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present disclosure are derived from, cancers including but not limited to primary or metastatic melanoma, thymoma, lymphoma, sarcoma, mesothelioma, renal cell carcinoma, stomach cancer, breast cancer, lung cancer, gastric cancer, ovarian cancer, NHL, leukemia, uterine cancer, prostate cancer, colon cancer, cervical cancer, bladder cancer, kidney cancer, brain cancer, liver cancer, pancreatic cancer, brain cancer, endometrial cancer, and stomach cancer.

The term “pharmaceutical composition” refers to a preparation which is in such form as to permit the biological activity of an active ingredient contained therein to be effective in treating a subject, and which contains no additional components which are unacceptably toxic to the subject in the amounts provided in the pharmaceutical composition.

The terms “modulate” and “modulation” refer to reducing or inhibiting or, alternatively, activating or increasing, a recited variable.

The terms “increase” and “activate” refer to an increase of 10%, 20%, 30%, 40%, 50%, 60%, 70%, 75%, 80%, 85%, 90%, 95%, 100%, 2-fold, 3-fold, 4-fold, 5-fold, 10-fold, 20-fold, 50-fold, 100-fold, or greater in a recited variable.

The terms “reduce” and “inhibit” refer to a decrease of 10%, 20%, 30%, 40%, 50%, 60%, 70%, 75%, 80%, 85%, 90%, 95%, 2-fold, 3-fold, 4-fold, 5-fold, 10-fold, 20-fold, 50-fold, 100-fold, or greater in a recited variable.

The term “agonize” refers to the activation of receptor signaling to induce a biological response associated with activation of the receptor. An “agonist” is an entity that binds to and agonizes a receptor.

The term “antagonize” refers to the inhibition of receptor signaling to inhibit a biological response associated with activation of the receptor. An “antagonist” is an entity that binds to and antagonizes a receptor.

The term “effector T cell” includes T helper (i.e., CD4+) cells and cytotoxic (i.e., CD8+) T cells. CD4+ effector T cells contribute to the development of several immunologic processes, including maturation of B cells into plasma cells and memory B cells, and activation of cytotoxic T cells and macrophages. CD8+ effector T cells destroy virus-infected cells and tumor cells. See Seder and Ahmed, Nature Immunol., 2003, 4:835-842, incorporated by reference in its entirety, for additional information on effector T cells.

The term “regulatory T cell” includes cells that regulate immunological tolerance, for example, by suppressing effector T cells. In some aspects, the regulatory T cell has a CD4+CD25+Foxp3+ phenotype. In some aspects, the regulatory T cell has a CD8+CD25+ phenotype. See Nocentini et al., Br. J. Pharmacol., 2012, 165:2089-2099, incorporated by reference in its entirety.

In some instances, the disease is a cancer selected from the group consisting of mesothelioma, papillary serous ovarian adenocarcinoma, clear cell ovarian carcinoma, mixed Mullerian ovarian carcinoma, endometroid mucinous ovarian carcinoma, malignant pleural disease, pancreatic adenocarcinoma, ductal pancreatic adenocarcinoma, uterine serous carcinoma, lung adenocarcinoma, extrahepatic bile duct carcinoma, gastric adenocarcinoma, esophageal adenocarcinoma, colorectal adenocarcinoma, breast adenocarcinoma, a disease associated with mesothelin expression, and combinations thereof, a disease associated with mesothelin expression, and combinations thereof.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, an antibody fragment or a specific ligand, which recognizes and binds a cognate binding partner (e.g., mesothelin) present in a sample, but which does not necessarily and substantially recognize or bind other molecules in the sample.

Ranges: throughout this disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Mesothelin

Mesothelin is a 40 kDa glycosyl-phosphatidyl inositol-linked membrane protein differentiation antigen, whose expression is mostly restricted to mesothelial cells lining the pleura, pericardium and peritoneum in healthy individuals (Chang and Pastan, 1996; Chang et al, 1992; Hassan and Ho, 2008). Mesothelin is overexpressed in multiple cancers, including more than 90% of malignant pleural mesotheliomas (MPMs) and pancreatic adenocarcinomas, approximately 70% of ovarian cancers, and approximately half of non-small cell lung cancers (NSCLCs), among others (Argani et al, 2001; Hassan and Ho, 2008; Hassan et al, 2005; Ordóñez, 2003). The precise physiological function of mesothelin is not completely understood, but it has been postulated to promote metastasis through its binding to MUC16 (Chen et al, 2013). MSLN (the gene encoding for mesothelin) knockout mice grow and reproduce normally and have no detectable phenotype. Therapeutic modalities include antibodies, recombinant immunotoxins, and CAR T cells. However, aberrant mesothelin expression plays an active role in both malignant transformation and tumor aggressiveness by promoting cancer cell proliferation, invasion, and metastasis.

Mesothelin expression is normally restricted to serosal cells of the pleura, peritoneum, and pericardium. Mesothelin is highly expressed in a wide range of solid tumors, including epitheloid mesothelioma (95%), extrahepatic biliary cancer (95%), pancreatic adenocarcinoma (85%), serous ovarian adenocarcinoma (75%), lung adenocarcinoma (57%), triple negative breast cancer (66%), endometrial carcinoma (59%), gastric carcinoma (47%), colorectal carcinoma (30%), and others.

Mesothelin overexpression is associated with poorer prognosis in mesothelioma, ovarian cancer, cholangiocarcinoma, lung adenocarcinoma, triple-negative breast cancer, and pancreatic adenocarcinoma.

Given its high expression in tumors and low expression in normal tissue, mesothelin is an attractive target for immunotherapy. Currently, several chimeric antigen receptor (CAR) T cell programs directed against mesothelin are being investigated.

The compositions and methods comprising anti-MSLN TFP T cells disclosed herein are a novel cell therapy that consists of genetically engineered T cells that express a single-domain antibody that recognizes human mesothelin fused to the CD3 subunit which, upon expression, is incorporated into the endogenous T cell receptor complex.

### Mesothelin Expression in Cancer

The expression of mesothelin in cancer has been broadly studied. Serial analyses of gene expression (SAGE: www.ncbi.nlm.nih.gov/projects/SAGE/), conducted at the National Institutes of Health (NIH), have shown high messenger ribonucleic acid (mRNA) expression of mesothelin in NSCLC, pancreatic cancer, MPM, ovarian cancer, cholangiocarcinoma, and other adenocarcinomas (Hassan and Ho, 2008). Expression profiles have been further supported by immunohistochemistry (IHC) studies performed on biopsy tissues taken from patients with multiple tumor types (Inaguma et al, 2017). While the IHC staining can vary dependent on antibody clone that is used, most IHC analyses indicate that 90% of ovarian cancer and >75% of MPM or pancreatic cancer biopsies are immunoreactive to anti-mesothelin antibodies. Mesothelin expression and prevalence in various tumor types has been reviewed by Morello et al (2016) (Table 1).

TABLE 1
Mesothelin Expression in Different Human Cancers
as Assessed by the Anti-mesothelin Antibody 5B2

| | | Mesothelin Expression | | | | | | | |
|---|---|---|---|---|---|---|---|---|---|
| | | (5B2 Anti-mesothelin Antibody) | | | | | | | |
| | Total | Luminal/Membrane | | | | Total | | | |
| Diagnosis | No. | No. | % | Range | | No. | % | Range | |
| Tumors with adenocarcinoma-like | | | | | | | | | |
| differentiation | | | | | | | | | |
| Ovary, serous carcinoma | 79 | 71 | 89.9 | 5-100 | (90) | 73 | 92.4 | 5-100 | (90) |
| Pancreas, invasive ductal | 132 | 93 | 70.5 | 5-100 | (50) | 100 | 75.8 | 5-100 | (80) |
| carcinoma | | | | | | | | | |
| Uterus, endometrioid | 82 | 52 | 63.4 | 5-100 | (20) | 62 | 75.6 | 5-100 | (30) |
| adenocarcinoma | | | | | | | | | |
| Colorectum, adenocarcinoma | 188 | 91 | 48.4 | 5-100 | (10) | 108 | 57.4 | 5-100 | (30) |
| Lung, adenocarcinoma | 76 | 36 | 47.4 | 5-100 | (30) | 50 | 65.8 | 5-100 | (80) |
| Liver, cholangiocellular | 39 | 16 | 41 | 10-100 | (60) | 16 | 41 | 20-100 | (95) |
| carcinoma | | | | | | | | | |
| Stomach, adenocarcinoma | 81 | 31 | 38.3 | 5-90 | (20) | 60 | 74.1 | 5-100 | (80) |
| Mammary gland, invasive ductal | 119 | 13 | 10.9 | 5-60 | (10) | 13 | 10.9 | 5-100 | (60) |
| carcinoma | | | | | | | | | |
| Prostate gland, adenocarcinoma | 107 | 0 | 0 | — | | 1 | 0.9 | 90 | (—) |
| Mammary gland, lobular | 82 | 0 | 0 | — | | 0 | 0 | — | |
| carcinoma | | | | | | | | | |
| Tumors with squamous cell | | | | | | | | | |
| differentiation | | | | | | | | | |
| Thymic carcinoma | 17 | 6 | 35.3 | 5-100 | (93) | 13 | 76.5 | 10-100 | (100) |
| Lung, squamous cell carcinoma | 72 | 20 | 27.8 | 5-90 | (10) | 33 | 45.8 | 5-100 | (20) |
| Uterine cervix, squamous cell | 21 | 3 | 14.3 | 20-40 | (20) | 6 | 28.6 | 20-100 | (90) |
| carcinoma | | | | | | | | | |
| Thymoma | 33 | 0 | 0 | — | | 0 | 0 | — | |
| Other epithelial tumors | | | | | | | | | |
| Malignant mesothelioma | 143 | 107 | 75 | 5-100 | (90) | 111 | 77.6 | 5-100 | (100) |
| Epithelioid | 115 | 93 | 80.9 | 5-100 | (90) | 97 | 84.3 | 5-100 | (100) |
| Biphasic | 17 | 11 | 64.7 | 20-100 | (70) | 11 | 64.7 | 20-100 | (100) |
| Sarcomatoid | 10 | 2 | 20 | 80 | (80) | 2 | 20 | 90-100 | (95) |
| Urinary tract, urothelial | 85 | 11 | 12.9 | 5-100 | (20) | 18 | 21.2 | 10-100 | (35) |
| carcinoma | | | | | | | | | |
| Kidney, clear cell renal cell | 206 | 9 | 4.4 | 5-100 | (20) | 15 | 7.3 | 5-100 | (50) |
| carcinoma | | | | | | | | | |

— = not applicable.

### Malignant Pleural Mesothelioma (MPM)

MPM represents about 80% of mesothelioma cases. MPM is a regional and highly aggressive tumor that arises from the mesothelium of the pleural surface. Rarely, other serosal membranes of the human body are also coated with mesothelium, such as peritoneum (peritoneal mesothelioma) and pericardium (pericardial mesothelioma), are affected. The incidence of MPM has increased significantly and it is estimated that 40,000 people die each year worldwide due to asbestos-related MPM. Different types of MPM have been identified including epithelioid (50%-70% of cases), biphasic (30%), and sarcomatoid (10%-20%) with increasingly aggressive behavior and worse prognosis. In addition to a high incidence (25%-60%) of somatic BAP1 mutations, MPM is also associated with frequent alterations in other major tumor suppressor genes, such as p16/Cdkn2a, p19/Arf, p19/Cdkn2b, and NF2. Effective treatment options for patients with MPM are very limited. The standard of care recommended for MPM is palliative chemotherapy with a doublet of platinum salt and an anti-folate. Unfortunately, objective response rates are 17% to 40% and the median overall survival (OS) of patients with MPM is 12 to 19 months when systemic chemotherapy is used with or without anti-angiogenic agents or targeted therapy. Anti-CTLA-4 failed to show a survival advantage as second-line therapy in MPM. Anti-programmed death receptor-1 (PD-1) and anti-PD-L1 antibodies (e.g., pembrolizumab, nivolumab, avelumab) are currently being tested in several trials in MPM. Early phase trials with anti-PD-1 or anti-PD-L1 antibodies have shown partial response rates up to 28% and disease control rates up to 76% with median duration of response of 12 months, but confirmatory data are required to validate these agents as the second line treatment of choice in MPM.

### Non-Small Cell Lung Cancer

NSCLC remains the leading cause of cancer-related mortality worldwide, accounting for approximately 18% of all cancer deaths. Despite treatment with platinum- and taxane-based chemotherapy, patients with metastatic NSCLC have a median survival of approximately 10 months, and a 5-year survival rate of approximately 15%. Despite the increased number of treatment options available for patients with non-squamous histology NSCLC, there has been little OS improvement from several new agents, including pemetrexed, erlotinib, and bevacizumab beyond very small subpopulations. Therapeutic options for mutation wild-type non-squamous NSCLC are particularly limited after failure of front-line chemotherapy. Overall, this group of patients only has an OS of about 8 months after progression from platinum agents. Once resistance to tyrosine kinase inhibitors (TKIs) occurs, the patients who have epidermal growth factor receptor (EGFR) mutations or ALK translocations will have a rapid disease progression. Therefore, NSCLC remains a disease with high unmet medical need. Recently, T cell checkpoint regulators such as CTLA-4 and programmed death-1 (PD-1, CD279) down-regulate T cell activation and proliferation upon engagement by their cognate ligands. T cell checkpoint inhibitors induce antitumor activity by breaking immune tolerance to tumor cell antigens. PD-1 and PD-L1 inhibitors are effective against metastatic NSCLC lacking sensitizing EGFR or ALK mutations.

Pembrolizumab (Keytruda, Merck), nivolumab (Opdivo, Bristol-Myers Squibb), and atezolizumab (Tecentriq, Genentech) are approved as second-line therapy. Among patients in whom the percentage of tumor cells with membranous PD-L1 staining (tumor proportion score) is 50% or greater, pembrolizumab has also replaced cytotoxic chemotherapy as the first-line treatment of choice. However, patients with a tumor proportion score of 50% or greater represent a minority of those with NSCLC. A randomized, phase 2 trial of carboplatin plus pemetrexed with or without pembrolizumab showed significantly better rates of response and longer progression-free survival (PFS) with the addition of pembrolizumab than with chemotherapy alone. In the global, double-blind, placebo-controlled, phase 3 KEYNOTE-189 trial, the addition of pembrolizumab to standard chemotherapy of pemetrexed and a platinum-based drug resulted in significantly longer OS and PFS than chemotherapy alone and such combination is likely to become standard frontline therapy (Ghandi et al, 2018). Notably, no standard of care is available for patients failing to respond or relapsing after checkpoint inhibitor therapy.

### Ovarian Cancer

Ovarian cancers can be classified in several subtypes according to their histopathology, which also determines their therapy. Epithelial ovarian cancer comprises 90% of all ovarian malignancies, with other pathologic subtypes such as germ cell and sex-cord stromal tumors being much rarer. It is estimated that 22,240 new diagnoses and 14,070 deaths from ovarian cancer will occur in 2018 in the United States (SEER, 2018). Ovarian cancer is characterized by late-stage presentation (more than 70% of cases), bulky metastatic tumor burden, and frequent recurrence of eventual chemoresistant disease, which result in cure rates below 15% among subjects with stage 3/4 disease. The 2 canonical types of drugs used to treat ovarian cancer—taxane and platinum-based agents—have not been replaced in the past 20 years, although the optimum timing of treatment (neoadjuvant versus adjuvant) and the best route of administration (intravenous versus intraperitoneal) remain unknown.

Recurrent ovarian cancer is not curable. The objectives of therapy are symptom palliation and extension of life. Subjects with platinum-sensitive ovarian cancer should be treated with a platinum-based agent. Those progressing after platinum retreatment and those with platinum-resistant disease, non-platinum combination and targeted therapies are available. The initial clinical efficacy of novel therapeutics, such as poly(ADP-ribose) polymerase (PARP) inhibitors and immune-checkpoint inhibitors, has ushered in a new wave of drug development in ovarian cancer. The synthetic lethality of BRCA mutated (ie, deficient) ovarian cancer cells exposed to the PARP inhibitor olaparib resulted in a median PFS of 7 months and median OS of 16.6 months. Efficacy with checkpoint inhibitors in subjects with advanced recurrent ovarian cancer has been modest thus far. Best overall response rate (ORR) has been 15% with nivolumab, 12% with pembrolizumab, and 10% with ipilimumab (Hamanishi et al, 2015; Varga et al, 2015).

### Cholangiocarcinoma

Cholangiocarcinomas are biliary epithelial tumors of the intrahepatic, perihilar, and distal biliary tree. Intrahepatic cholangiocarcinomas (iCCAs) (20% of cases) arise above the second-order bile ducts, whereas the cystic duct is the anatomical point of distinction between perihilar cholangiocarcinomas (pCCAs) (50%-60%), and distal cholangiocarcinomas (dCCAs; 20-30%). Most subjects have advanced-stage disease at presentation due to its aggressiveness and difficulty in early diagnosis. While surgery is the preferred therapy, only 35% of cases have early disease amenable to surgical resection with curative intent. For unresectable cholangiocarcinoma, the available standard-of-care chemotherapy (gemcitabine and cisplatin) renders a median OS<1 year, partly due to the desmoplastic stroma that fosters cancer cell survival and poses a barrier to the delivery of chemotherapy. Recurrent mutations in IDH1, IDH2, FGFR1, FGFR2, FGFR3, EPHA2, and BAP1 are found predominantly in iCCAs, whereas ARID1B, ELF3, PBRM1, PRKACA, and PRKACB mutations occur preferentially in pCCA/dCCA. Some of the latter represent actionable mutations whose therapeutic potential is currently being investigated in clinical trials. At present, clinical data on immunotherapy in cholangiocarcinoma are limited. PD-L1 expression has been reported in 9% to 72% of specimens, and on 46% to 63% of immune cells within the tumor microenvironment. Interim data from the KEYNOTE-028 basket trial (NCT02054806) with pembrolizumab have been reported. Of the 24 enrolled subjects with PD-L1 expression ≥1% (20 cholangiocarcinoma, 4 gallbladder carcinoma), 4 (17%, 3 with cholangiocarcinoma and 1 with gallbladder carcinoma) had a partial response (PR), and 4 (17%) had stable disease (SD). The median PFS was not reached at the time of reporting. These data prompted the launching of a biliary cancer cohort of 100 subjects in the ongoing KEYNOTE-158 basket trial (NCT02628067).

### T Cell Receptor (TCR) Fusion Proteins (TFPs)

The present disclosure encompasses DNA and RNA constructs encoding TFPs, and variants thereof, wherein the TFP comprises a binding domain, e.g., an antibody or an antibody fragment, a ligand, or a ligand binding protein, that binds specifically to a tumor-associated antigen e.g., mesothelin, e.g., human mesothelin, wherein the sequence of the antibody fragment is contiguous with and in the same reading frame as a nucleic acid sequence encoding a TCR subunit or portion thereof. The TFPs are able to associate with one or more endogenous (or alternatively, one or more exogenous, or a combination of endogenous and exogenous) TCR subunits in order to form a functional TCR complex.

The TFPs can comprise a target-specific binding element otherwise referred to as an antigen binding domain. The choice of moiety depends upon the type and number of target antigen that define the surface of a target cell. For example, the antigen binding domain may be chosen to recognize a target antigen that acts as a cell surface marker on target cells associated with a particular disease state. Thus, examples of cell surface markers that may act as target antigens for the antigen binding domain in a TFP of the disclosure include those associated with viral, bacterial and parasitic infections; autoimmune diseases; and cancerous diseases (e.g., malignant diseases).

The TFP-mediated T cell response can be directed to an antigen of interest by way of engineering an antigen-binding domain into the TFP that specifically binds a desired antigen. A portion of the TFP may comprise the antigen binding domain that targets mesothelin.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (V_{H}), a light chain variable domain (V_{L}) and a variable domain (V_{HH}) of a camelid derived nanobody, and to an alternative scaffold known in the art to function as antigen binding domain, such as a recombinant fibronectin domain, anticalin, DARPIN and the like. Likewise a natural or synthetic ligand specifically recognizing and binding the target antigen can be used as antigen binding domain for the TFP. In some instances, it is beneficial for the antigen binding domain to be derived from the same species in which the TFP will ultimately be used in. For example, for use in humans, it may be beneficial for the antigen binding domain of the TFP to comprise human or humanized residues for the antigen binding domain of an antibody or antibody fragment.

Thus, the antigen-binding domain can comprise a humanized or human antibody or an antibody fragment, or a murine antibody or antibody fragment. The humanized or human anti-mesothelin binding domain may comprise one or more (e.g., all three) light chain complementary determining region 1 (LC CDR1), light chain complementary determining region 2 (LC CDR2), and light chain complementary determining region 3 (LC CDR3) of a humanized or human anti-mesothelin binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-mesothelin binding domain described herein, e.g., a humanized or human anti-mesothelin binding domain comprising one or more, e.g., all three, LC CDRs and one or more, e.g., all three, HC CDRs. The humanized or human anti-mesothelin binding domain may comprise one or more (e.g., all three) heavy chain complementary determining region 1 (HC CDR1), heavy chain complementary determining region 2 (HC CDR2), and heavy chain complementary determining region 3 (HC CDR3) of a humanized or human anti-mesothelin binding domain described herein, e.g., the humanized or human anti-mesothelin binding domain has two variable heavy chain regions, each comprising a HC CDR1, a HC CDR2 and a HC CDR3 described herein. The humanized or human anti-mesothelin binding domain may comprise a humanized or human light chain variable region described herein and/or a humanized or human heavy chain variable region described herein. The humanized or human anti-mesothelin binding domain may comprise a humanized heavy chain variable region described herein, e.g., at least two humanized or human heavy chain variable regions described herein. The anti-mesothelin binding domain can be a scFv comprising a light chain and a heavy chain of an amino acid sequence provided herein. The anti-mesothelin binding domain can be a VHH comprising a heavy chain of an amino acid sequence provided herein. The anti-mesothelin binding domain (e.g., a scFv or VHH) may comprise: a light chain variable region comprising an amino acid sequence having at least one, two or three modifications (e.g., substitutions) but not more than 30, 20 or 10 modifications (e.g., substitutions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided herein, or a sequence with 95-99% identity to an amino acid sequence provided herein. The humanized or human anti-mesothelin binding domain can be a scFv, and a light chain variable region comprising an amino acid sequence described herein, is attached to a heavy chain variable region comprising an amino acid sequence described herein, via a linker, e.g., a linker described herein. The humanized anti-mesothelin binding domain may include a (Gly₄-Ser)ₙ linker, wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region. In some instances, the linker sequence comprises a long linker (LL) sequence. In some instances, the long linker sequence comprises (G₄S)ₙ, wherein n=2 to 4. In some instances, the linker sequence comprises a short linker (SL) sequence. In some instances, the short linker sequence comprises (G₄S)ₙ, wherein n=1 to 3.

A non-human antibody may be humanized, where specific sequences or regions of the antibody are modified to increase similarity to an antibody naturally produced in a human or fragment thereof.

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature, 332:323, which are incorporated herein by reference in their entireties).

A humanized antibody or antibody fragment has one or more amino acid residues remaining in it from a source which is nonhuman. These nonhuman amino acid residues are often referred to as “import” residues, which are typically taken from an “import” variable domain. Humanized antibodies or antibody fragments may comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). The framework region, e.g., all four framework regions, of the heavy chain variable region may be derived from a V_{H}4-4-59 germline sequence. The framework region can comprise, one, two, three, four or five modifications, e.g., substitutions, e.g., from the amino acid at the corresponding murine sequence. The framework region, e.g., all four framework regions of the light chain variable region may be derived from a VK3-1.25 germline sequence. The framework region can comprise, one, two, three, four or five modifications, e.g., substitutions, e.g., from the amino acid at the corresponding murine sequence.

The portion of a TFP composition that comprises an antibody fragment can be humanized with retention of high affinity for the target antigen and other favorable biological properties. Humanized antibodies and antibody fragments may be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present disclosure, the ability to bind human mesothelin. A humanized antibody or antibody fragment may have improved affinity and/or specificity of binding to human mesothelin.

The anti-mesothelin binding domain can be characterized by particular functional features or properties of an antibody or antibody fragment. For example, the portion of a TFP composition of the disclosure that comprises an antigen binding domain can specifically bind human mesothelin. The antigen binding domain has the same or a similar binding specificity to human mesothelin as the FMC63 scFv described in Nicholson et al. Mol. Immun. 34 (16-17): 1157-1165 (1997). The disclosure can relate to an antigen binding domain comprising an antibody or antibody fragment, wherein the antibody binding domain specifically binds to a mesothelin protein or fragment thereof, wherein the antibody or antibody fragment comprises a variable light chain and/or a variable heavy chain that includes an amino acid sequence provided herein. The scFv may be contiguous with and in the same reading frame as a leader sequence.

### Anti-MSLN TFP T Cells

### Stability and Mutations

The stability of an anti-mesothelin binding domain, e.g., sdAb or scFv molecules (e.g., soluble sdAb or scFv) can be evaluated in reference to the biophysical properties (e.g., thermal stability) of a conventional control scFv molecule or a full length antibody. The humanized or human sdAb or scFv may have a thermal stability that is greater than about 0.1, about 0.25, about 0.5, about 0.75, about 1, about 1.25, about 1.5, about 1.75, about 2, about 2.5, about 3, about 3.5, about 4, about 4.5, about 5, about 5.5, about 6, about 6.5, about 7, about 7.5, about 8, about 8.5, about 9, about 9.5, about 10 degrees, about 11 degrees, about 12 degrees, about 13 degrees, about 14 degrees, or about 15 degrees Celsius than a parent sdAb or scFv in the described assays.

The improved thermal stability of the anti-mesothelin binding domain, e.g., sdAb or scFv is subsequently conferred to the entire mesothelin-TFP construct, leading to improved therapeutic properties of the anti-mesothelin TFP construct. The thermal stability of the anti-mesothelin binding domain, e.g., sdAb or scFv can be improved by at least about 2° C. or 3° C. as compared to a conventional antibody. The anti-mesothelin binding domain, e.g., sdAb or scFv may have a 1° C., 2° C., 3° C., 4° C., 5° C., 6° C., 7° C., 8° C., 9° C., 10° C., 11° C., 12° C., 13° C., 14° C., or 15° C. improved thermal stability as compared to a conventional antibody. Comparisons can be made, for example, between the sdAb or scFv molecules disclosed herein and sdAb or scFv molecules or Fab fragments of an antibody from which the sdAb V_{HH} was derived or the scFv V_{H} and V_{L} were derived. Thermal stability can be measured using methods known in the art. For example, T_{M} can be measured. Methods for measuring T_{M} and other methods of determining protein stability are described below.

Mutations in sdAb or scFv (arising through humanization or mutagenesis of the soluble sdAb or scFv) alter the stability of the sdAb or scFv and improve the overall stability of the sdAb or scFv and the anti-mesothelin TFP construct. Stability of the humanized scFv is compared against the llama sdAb or murine scFv using measurements such as T_{M}, temperature denaturation and temperature aggregation. The anti-mesothelin binding domain, e.g., a sdAb or scFv, may comprise at least one mutation arising from the humanization process such that the mutated sdAb or scFv confers improved stability to the anti-mesothelin TFP construct. The anti-mesothelin binding domain, e.g., sdAb or scFv may comprise at least 1, 2, 3, 4, 5, 6, 7, 8, 9, 10 mutations arising from the humanization process such that the mutated sdAb or scFv confers improved stability to the mesothelin-TFP construct.

The antigen binding domain of the TFP may comprise an amino acid sequence that is homologous to an antigen binding domain amino acid sequence described herein, and the antigen binding domain retains the desired functional properties of the anti-mesothelin antibody fragments described herein. The TFP composition of the disclosure may comprise an antibody fragment, e.g., a sdAb or scFv.

The antigen binding domain of the TFP can be engineered by modifying one or more amino acids within one or both variable regions (e.g., V_{HH}, V_{H} and/or V_{L}), for example within one or more CDR regions and/or within one or more framework regions. The TFP composition of the disclosure may comprise an antibody fragment, e.g., a sdAb or scFv.

It will be understood by one of ordinary skill in the art that the antibody or antibody fragment of the TFP may further be modified such that they vary in amino acid sequence (e.g., from wild-type), but not in desired activity. For example, additional nucleotide substitutions leading to amino acid substitutions at “non-essential” amino acid residues may be made to the protein. For example, a nonessential amino acid residue in a molecule may be replaced with another amino acid residue from the same side chain family. A string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences refers to two or more sequences that are the same. Two sequences are “substantially identical” if two sequences have a specified percentage of amino acid residues or nucleotides that are the same (e.g., 60% identity, optionally 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology). Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The present disclosure contemplates modifications of the starting antibody or fragment (e.g., sdAb or scFv) amino acid sequence that generate functionally equivalent molecules. For example, the V_{HH} and V_{H} or V_{L} of an anti-mesothelin binding domain, e.g., sdAb or scFv, comprised in the TFP can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting V_{HH} and V_{H} or V_{L} framework region of the anti-mesothelin binding domain, e.g., sdAb or scFv. The present disclosure contemplates modifications of the entire TFP construct, e.g., modifications in one or more amino acid sequences of the various domains of the TFP construct in order to generate functionally equivalent molecules. The TFP construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity of the starting TFP construct.

### Extracellular Domain

The extracellular domain may be derived either from a natural or from a recombinant source. Where the source is natural, the domain may be derived from any protein, but in particular a membrane-bound or transmembrane protein. The extracellular domain is capable of associating with the transmembrane domain. An extracellular domain of particular use in this disclosure may include at least the extracellular region(s) of e.g., the alpha, beta or zeta chain of the T cell receptor, or CD3 epsilon, CD3 gamma, or CD3 delta, or, alternatively, CD28, CD45, CD4, CD5, CD8, CD9, CD16, CD22, CD33, CD37, CD64, CD80, CD86, CD134, CD137, CD154.

### Transmembrane Domain

In general, a TFP sequence contains an extracellular domain and a transmembrane domain encoded by a single genomic sequence. A TFP can be designed to comprise a transmembrane domain that is heterologous to the extracellular domain of the TFP. A transmembrane domain can include one or more additional amino acids adjacent to the transmembrane region, e.g., one or more amino acid associated with the extracellular region of the protein from which the transmembrane was derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the extracellular region) and/or one or more additional amino acids associated with the intracellular region of the protein from which the transmembrane protein is derived (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids of the intracellular region). In some cases, the transmembrane domain can include at least 30, 35, 40, 45, 50, 55, 60 or more amino acids of the extracellular region. In some cases, the transmembrane domain can include at least 30, 35, 40, 45, 50, 55, 60 or more amino acids of the intracellular region). The transmembrane domain is one that is associated with one of the other domains of the TFP is used. In some instances, the transmembrane domain can be selected or modified by amino acid substitution to avoid binding of such domains to the transmembrane domains of the same or different surface membrane proteins, e.g., to minimize interactions with other members of the receptor complex. The transmembrane domain may be capable of homodimerization with another TFP on the TFP-T cell surface. Alternatively, the amino acid sequence of the transmembrane domain may be modified or substituted so as to minimize interactions with the binding domains of the native binding partner present in the same TFP.

The transmembrane domain may be derived either from a natural or from a recombinant source. Where the source is natural, the domain may be derived from any membrane-bound or transmembrane protein. The transmembrane domain may be capable of signaling to the intracellular domain(s) whenever the TFP has bound to a target. A transmembrane domain of particular use in this disclosure may include at least the transmembrane region(s) of e.g., the alpha, beta, gamma, delta, or zeta chain of the T cell receptor, CD28, CD3 epsilon, CD3 gamma, CD3 delta, CD45, CD4, CD5, CD8, CD9, CD16, CD22, CD33, CD37, CD64, CD80, CD86, CD134, CD137, CD154.

In some instances, the transmembrane domain can be attached to the extracellular region of the TFP, e.g., the antigen binding domain of the TFP, via a hinge, e.g., a hinge from a human protein. For example, the hinge can be a human immunoglobulin (Ig) hinge, e.g., an IgG4 hinge, or a CD8a hinge.

### Linkers

Optionally, a short oligo- or polypeptide linker, between 2 and 10 amino acids in length may form the linkage between the transmembrane domain and the cytoplasmic region of the TFP. In some cases, the linker may be at least about 5. 6. 7. 8. 9. 10, 11, 12, 13, 14, 15, 16, 17, 18, 19, 20 or more in length. A glycine-serine doublet provides a particularly suitable linker. For example, the linker comprises the amino acid sequence of GGGGSGGGGS (SEQ ID NO. 53). The linker can be encoded by a nucleotide sequence of GGTGGCGGAGGTTCTGGAGGTGGAGGTTCC (SEQ ID NO. 54).

### Cytoplasmic Domain

The cytoplasmic domain of the TFP can include an intracellular domain. In some embodiments, if the TFP contains CD3 gamma, delta or epsilon polypeptides, the TFP comprises an intracellular signaling domain; the intracellular subunits of TCR alpha and TCR beta are generally lacking in a signaling domain, but are able to recruit CD3 zeta which comprises an intracellular signaling domain. An intracellular signaling domain is generally responsible for activation of at least one of the normal effector functions of the immune cell in which the TFP has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the TFP of the disclosure include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of naive T cells and that a secondary and/or costimulatory signal is required. Thus, naïve T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs (ITAMs).

Examples of ITAMs containing primary intracellular signaling domains that are of particular use in the disclosure include those of CD3 zeta, FcR gamma, FcR beta, CD3 gamma, CD3 delta, CD3 epsilon, CD5, CD22, CD79a, CD79b, and CD66d. The TFP used in the disclosure may comprise an intracellular signaling domain, e.g., a primary signaling domain of CD3-epsilon. A primary signaling domain may comprise a modified ITAM domain, e.g., a mutated ITAM domain which has altered (e.g., increased or decreased) activity as compared to the native ITAM domain. A primary signaling domain may comprise a modified ITAM-containing primary intracellular signaling domain, e.g., an optimized and/or truncated ITAM-containing primary intracellular signaling domain. A primary signaling domain may comprise one, two, three, four or more ITAM motifs.

The intracellular signaling domain of the TFP can comprise the CD3 epsilon signaling domain by itself or it can be combined with any other desired intracellular signaling domain(s) useful in the context of a TFP of the disclosure. For example, the intracellular signaling domain of the TFP can comprise a CD3 epsilon chain portion and a costimulatory signaling domain. The costimulatory signaling domain refers to a portion of the TFP comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, DAP10, DAP12, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human TFP-T cells in vitro and augments human T cell persistence and antitumor activity in vivo (Song et al. Blood. 2012; 119(3):696-706).

The intracellular signaling sequences within the cytoplasmic portion of the TFP of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences.

A glycine-serine doublet can be used as a suitable linker, or alternatively, single amino acid, e.g., an alanine, a glycine, can be used as a suitable linker.

The TFP-expressing cell described herein can further comprise a second TFP, e.g., a second TFP that includes a different antigen binding domain, e.g., to the same target (mesothelin) or a different target (e.g., CD123). When the TFP-expressing cell can comprise two or more different TFPs, the antigen binding domains of the different TFPs can be such that the antigen binding domains do not interact with one another. For example, a cell expressing a first and second TFP can have an antigen binding domain of the first TFP, e.g., as a fragment, e.g., a scFv, that does not associate with the antigen binding domain of the second TFP, e.g., the antigen binding domain of the second TFP is a V_{HH}.

The TFP-expressing cell described herein can further express another agent, e.g., an agent which enhances the activity of a TFP-expressing cell. For example, the agent can be an agent which inhibits an inhibitory molecule. Inhibitory molecules, e.g., PD1, can decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. The agent that inhibits an inhibitory molecule may comprise a first polypeptide, e.g., an inhibitory molecule, associated with a second polypeptide that provides a positive signal to the cell, e.g., an intracellular signaling domain described herein. The agent may comprise a first polypeptide, e.g., of an inhibitory molecule such as PD1, LAG3, CTLA4, CD160, BTLA, LAIR1, TIM3, 2B4 and TIGIT, or a fragment of any of these (e.g., at least a portion of an extracellular domain of any of these), and a second polypeptide which is an intracellular signaling domain described herein (e.g., comprising a costimulatory domain (e.g., 4-1BB, CD27 or CD28, e.g., as described herein) and/or a primary signaling domain (e.g., a CD3 zeta signaling domain described herein). The agent may comprise a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

The agent may comprise the extracellular domain (ECD) of an inhibitory molecule, e.g., Programmed Death 1 (PD1) can be fused to a transmembrane domain and optionally an intracellular signaling domain such as 41BB and CD3 zeta (also referred to herein as a PD1 TFP). The PD1 TFP, when used in combinations with an anti-mesothelin TFP described herein, may improve the persistence of the T cell. The TFP may be a PD1 TFP comprising the extracellular domain of PD 1. Alternatively, TFPs may contain an antibody or antibody fragment such as a sdAb or scFv that specifically binds to the Programmed Death-Ligand 1 (PD-L1) or Programmed Death-Ligand 2 (PD-L2).

The present disclosure provides methods of administering a population of TFP-expressing T cells, e.g., TFP-T cells. The population of TFP-expressing T cells may comprise a mixture of cells expressing different TFPs. For example, the population of TFP-T cells can include a first cell expressing a TFP having an anti-mesothelin binding domain described herein, and a second cell expressing a TFP having a different anti-mesothelin binding domain, e.g., an anti-mesothelin binding domain described herein that differs from the anti-mesothelin binding domain in the TFP expressed by the first cell. As another example, the population of TFP-expressing cells can include a first cell expressing a TFP that includes an anti-mesothelin binding domain, e.g., as described herein, and a second cell expressing a TFP that includes an antigen binding domain to a target other than mesothelin (e.g., another tumor-associated antigen).

The present disclosure also provides methods of administration of a population of cells wherein at least one cell in the population expresses a TFP having an anti-mesothelin domain described herein, and a second cell expressing another agent, e.g., an agent which enhances the activity of a TFP-expressing cell. For example, the agent can be an agent which inhibits an inhibitory molecule. Inhibitory molecules, e.g., can, decrease the ability of a TFP-expressing cell to mount an immune effector response. Examples of inhibitory molecules include PD1, PD-L1, PD-L2, CTLA4, TIM3, LAG3, VISTA, BTLA, TIGIT, LAIR1, CD160, 2B4 and TGFR beta. The agent that inhibits an inhibitory molecule may comprise a first polypeptide, e.g., an inhibitory molecule, associated with a second polypeptide that provides a positive signal to the cell, e.g., an intracellular signaling domain described herein.

Disclosed herein are methods for producing in vitro transcribed RNA encoding TFPs. The present disclosure also includes a TFP encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length. RNA so produced can efficiently transfect different kinds of cells. The template may include sequences for the TFP.

The anti-mesothelin TFP is encoded by a messenger RNA (mRNA). The mRNA encoding the anti-mesothelin TFP may be introduced into a T cell for production of a TFP-T cell. The in vitro transcribed RNA TFP can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is a TFP of the present disclosure. The DNA to be used for PCR may contain an open reading frame. The DNA can be from a naturally occurring DNA sequence from the genome of an organism. The nucleic acid can include some or all of the 5′ and/or 3′ untranslated regions (UTRs). The nucleic acid can include exons and introns. The DNA to be used for PCR can be a human nucleic acid sequence, optionally including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. The primers may be designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5′ to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA preferably has 5′ and 3′ UTRs. The 5′ UTR can be between one and 3,000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′UTR sequences can decrease the stability of mRNA. Therefore, 3′ UTRs can be selected or designed to increase the stability of the transcribed RNA based on properties of UTRs that are well known in the art.

The 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. The 5′ UTR can be 5′UTR of an RNA virus whose RNA genome is stable in cells. Various nucleotide analogues can be used in the 3′ or 5′ UTR to impede exonuclease degradation of the mRNA.

To enable synthesis of RNA from a DNA template without the need for gene cloning, a promoter of transcription is attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. The promoter can be a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

The mRNA can have both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100 T tail (size can be 50-5000 Ts), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. The poly(A) tail can be between 100 and 5000 adenosines.

Poly(A) tails of RNAs can be further extended following in vitro transcription with the use of a poly(A) polymerase, such as E. coli polyA polymerase (E-PAP). Increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. RNAs produced by the methods disclosed herein may include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Gene Editing of TCR Complex or Endogenous Protein-Coding Genes

In some embodiments, the modified T cells disclosed herein are engineered using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. In this way, a chimeric construct may be engineered to combine desirable characteristics of each subunit, such as conformation or signaling capabilities. See also Sander & Joung, Nat. Biotech. (2014) v32, 347-55; and June et al., 2009 Nature Reviews Immunol. 9.10: 704-716, each incorporated herein by reference. In some embodiments, one or more of the extracellular domain, the transmembrane domain, or the cytoplasmic domain of a TFP subunit are engineered to have aspects of more than one natural TCR subunit domain (i.e., are chimeric).

Recent developments of technologies to permanently alter the human genome and to introduce site-specific genome modifications in disease relevant genes lay the foundation for therapeutic applications. These technologies are now commonly known as “genome editing.

In some embodiments, gene editing techniques are employed to disrupt an endogenous TCR gene. In some embodiments, mentioned endogenous TCR gene encodes a TCR gamma chain, a TCR delta chain, or a TCR gamma chain and a TCR delta chain. In some embodiments, gene editing techniques pave the way for multiplex genomic editing, which allows simultaneous disruption of multiple genomic loci in endogenous TCR gene. In some embodiments, multiplex genomic editing techniques are applied to generate gene-disrupted T cells that are deficient in the expression of endogenous TCR, and/or human leukocyte antigens (HLAs), and/or programmed cell death protein 1 (PD1), and/or other genes.

Current gene editing technologies comprise meganucleases, zinc-finger nucleases (ZFN), TAL effector nucleases (TALEN), and clustered regularly interspaced short palindromic repeats (CRISPR)/CRISPR-associated (Cas) system. These four major classes of gene-editing techniques share a common mode of action in binding a user-defined sequence of DNA and mediating a double-stranded DNA break (DSB). DSB may then be repaired by either non-homologous end joining (NHEJ) or—when donor DNA is present-homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental means to produce a protein that binds to a pre-determined DNA sequence −18 basepairs in length. By fusing this engineered protein domain to the Fokl nuclease, it is possible to target DNA breaks with genome-level specificity. ZFNs have been used extensively to target gene addition, removal, and substitution in a wide range of eukaryotic organisms (reviewed in Durai et al. (2005) Nucleic Acids Res 33, 5978). Likewise, TAL-effector nucleases (TALENs) can be generated to cleave specific sites in genomic DNA. Like a ZFN, a TALEN comprises an engineered, site-specific DNA-binding domain fused to the Fokl nuclease domain (reviewed in Mak et al. (2013), Curr Opin Struct Biol. 23:93-9). In this case, however, the DNA binding domain comprises a tandem array of TAL-effector domains, each of which specifically recognizes a single DNA basepair. Compact TALENs have an alternative endonuclease architecture that avoids the need for dimerization (Beurdeley et al. (2013), Nat Commun. 4: 1762). A Compact TALEN comprises an engineered, site-specific TAL-effector DNA-binding domain fused to the nuclease domain from the I-Teel homing endonuclease. Unlike Fokl, I-Teel does not need to dimerize to produce a double-strand DNA break so a Compact TALEN is functional as a monomer.

Engineered endonucleases based on the CRISPR/Cas9 system are also known in the art (Ran et al. (2013), Nat Protoc. 8:2281-2308; Mali et al. (2013), Nat Methods 10:957-63). The CRISPR gene-editing technology is composed of an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or a RNA duplex comprising a 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems known in the art (Adli (2018) Nat. Commun. 9:1911), each containing multiple CRISPR types. Class 1 contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed by researchers for genome editing. More than 10 different CRISPR/Cas proteins have been remodeled within last few years (Adli (2018) Nat. Commun. 9:1911). Among these, such as Cas12a (Cpf1) proteins from Acid-aminococcus sp (AsCpf1) and Lachnospiraceae bacterium (LbCpf1), are particularly interesting.

Homing endonucleases are a group of naturally occurring nucleases that recognize 15-40 basepair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38: 49-95). Specific amino acid substations could reprogram DNA cleavage specificity of homing nucleases (Niyonzima (2017), Protein Eng Des Sel. 30(7): 503-522). Meganucleases (MN) are monomeric proteins with innate nuclease activity that are derived from bacterial homing endonucleases and engineered for a unique target site (Gersbach (2016), Molecular Therapy. 24: 430-446). In some embodiments, meganuclease is engineered I-CreI homing endonuclease. In other embodiments, meganuclease is engineered I-SceI homing endonuclease.

In addition to mentioned four major gene editing technologies, chimeric proteins comprising fusions of meganucleases, ZFNs, and TALENs have been engineered to generate novel monomeric enzymes that take advantage of the binding affinity of ZFNs and TALENs and the cleavage specificity of meganucleases (Gersbach (2016), Molecular Therapy. 24: 430-446). For example, A megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, must be efficiently delivered to the cells of interest. Delivery methods such as physical, chemical, and viral methods are also know in the art (Mali (2013). Indian J. Hum. Genet. 19: 3-8). In some instances, physical delivery methods can be selected from the methods but not limited to electroporation, microinjection, or use of ballistic particles. On the other hand, chemical delivery methods require use of complex molecules such calcium phosphate, lipid, or protein. In some embodiments, viral delivery methods are applied for gene editing techniques using viruses such as but not limited to adenovirus, lentivirus, and retrovirus.

### Vectors

In some embodiments, the instant disclosure provides vectors comprising the recombinant nucleic acid(s) encoding the TFP and/or additional molecules of interest (e.g., a protein or proteins to be secreted by the TFP T cell). In some instances, the vector is selected from the group consisting of a DNA, a RNA, a plasmid, a lentivirus vector, adenoviral vector, an adeno-associated viral vector (AAV), a Rous sarcoma viral (RSV) vector, or a retrovirus vector. In some instances, the vector is an AAV6 vector. In some instances, the vector further comprises a promoter. In some instances, the vector is an in vitro transcribed vector.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The present disclosure also provides vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

The vector comprising the nucleic acid encoding the desired TFP of the disclosure can be an adenoviral vector (A5/35). The expression of nucleic acids encoding TFPs can be accomplished using of transposons such as sleeping beauty, crisper, CAS9, and zinc finger nucleases (See, June et al. 2009 Nature Reviews Immunol. 9.10: 704-716, incorporated herein by reference).

The expression constructs of the present disclosure may also be used for nucleic acid immunization and gene therapy, using standard gene delivery protocols. Methods for gene delivery are known in the art (see, e.g., U.S. Pat. Nos. 5,399,346, 5,580,859, 5,589,466, incorporated by reference herein in their entireties).

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, e.g., in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

A number of virally based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. Adenovirus vectors can be used. A number of adenovirus vectors are known in the art. Lentivirus vectors can also be used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a TFP transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving TFP expression from transgenes cloned into a lentiviral vector (see, e.g., Milone et al., Mol. Ther. 17(8): 1453-1464 (2009)). Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1a promoter, the hemoglobin promoter, and the creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline-regulated promoter.

In order to assess the expression of a TFP polypeptide or portions thereof, the expression vector to be introduced into a cell can also contain either a selectable marker gene or a reporter gene or both to facilitate identification and selection of expressing cells from the population of cells sought to be transfected or infected through viral vectors. The selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art (see, e.g., Sambrook et al., 2012, Molecular Cloning: A Laboratory Manual, volumes 1-4, Cold Spring Harbor Press, NY). One method for the introduction of a polynucleotide into a host cell is calcium phosphate transfection

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like (see, e.g., U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). The nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

The present disclosure further provides a vector comprising a TFP encoding nucleic acid molecule. A TFP vector can be directly transduced into a cell, e.g., a T cell. The vector may be a cloning or expression vector, e.g., a vector including, but not limited to, one or more plasmids (e.g., expression plasmids, cloning vectors, minicircles, minivectors, double minute chromosomes), retroviral and lentiviral vector constructs. The vector may be capable of expressing the TFP construct in mammalian T cells, e.g., a human T cell.

### Therapeutic Applications

The TFP T cells provided herein may be useful for the treatment of any disease or condition involving mesothelin over-expression. In some embodiments, the disease or condition is a disease or condition that can benefit from treatment with adoptive cell therapy. In some embodiments, the disease or condition is a tumor. In some embodiments, the disease or condition is a cell proliferative disorder. In some embodiments, the disease or condition is a cancer.

In some embodiments, provided herein is a method of treating a disease or condition in a subject in need thereof by administering an effective amount of a TFP T cell provided herein to the subject. In some aspects, the disease or condition is a cancer.

Any suitable cancer may be treated with the TFP T cells provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

### Modified T Cells

Disclosed herein, in some embodiments, are modified T cells comprising the recombinant nucleic acid disclosed herein, or the vectors disclosed herein; wherein the modified T cell comprises a functional disruption of an endogenous TCR. Also disclosed herein, in some embodiments, are modified T cells comprising the sequence encoding the TFP of the nucleic acid disclosed herein or a TFP encoded by the sequence of the nucleic acid disclosed herein, wherein the modified T cell comprises a functional disruption of an endogenous TCR. Further disclosed herein, in some embodiments, are modified allogenic T cells comprising the sequence encoding the TFP disclosed herein or a TFP encoded by the sequence of the nucleic acid disclosed herein.

In some instances, the T cell further comprises a heterologous sequence encoding a TCR constant domain, wherein the TCR constant domain is a TCR gamma constant domain, a TCR delta constant domain or a TCR gamma constant domain and a TCR delta constant domain. In some instances, the endogenous TCR that is functionally disrupted is an endogenous TCR gamma chain, an endogenous TCR delta chain, or an endogenous TCR gamma chain and an endogenous TCR delta chain. In some instances, the endogenous TCR that is functionally disrupted has reduced binding to WIC-peptide complex compared to that of an unmodified control T cell. In some instances, the functional disruption is a disruption of a gene encoding the endogenous TCR. In some instances, the disruption of a gene encoding the endogenous TCR is a removal of a sequence of the gene encoding the endogenous TCR from the genome of a T cell. In some instances, the T cell is a human T cell. In some instances, the T cell is a CD8+ or CD4+ T cell. In some instances, the T cell is an allogenic T cell. In some instances, the T cell is a TCR alpha-beta T cell. In some instances, the T cell is a TCR gamma-delta T cell. In some instances, one or more of TCR alpha, TCR beta, TCR gamma, and TCR delta have been modified to produce an allogeneic T cell. See, e.g., copending PCT Publication No. WO2019173693, which is herein incorporated by reference.

In some instances, the modified T cells further comprise a nucleic acid encoding an inhibitory molecule that comprises a first polypeptide comprising at least a portion of an inhibitory molecule, associated with a second polypeptide comprising a positive signal from an intracellular signaling domain. In some instances, the inhibitory molecule comprises the first polypeptide comprising at least a portion of PD1 and the second polypeptide comprising a costimulatory domain and primary signaling domain.

### Sources of T Cells

Prior to expansion and genetic modification, a source of T cells is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. Any number of T cell lines available in the art, may be used. T cells can be obtained from a unit of blood collected from a subject using any number of techniques known to the skilled artisan, such as Ficoll™ separation. Cells from the circulating blood of an individual are typically obtained by apheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. The cells may be washed with phosphate buffered saline (PBS). The wash solution may lack calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

T cells can be isolated from peripheral blood lymphocytes by lysing the red blood cells and depleting the monocytes, for example, by centrifugation through a PERCOLL® gradient or by counterflow centrifugal elutriation. A specific subpopulation of T cells, such as CD3+, CD28+, CD4+, CD8+, CD45RA+, and CD45RO+, alpha-beta, or, gamma-delta T cells, can be further isolated by positive or negative selection techniques. For example, T cells can be isolated by incubation with anti-CD3/anti-CD28 (e.g., 3×28)-conjugated beads, such as DYNABEADS® M-450 CD3/CD28 T, for a time period sufficient for positive selection of the desired T cells. The time period can be about 30 minutes. The time period may range from 30 minutes to 36 hours or longer and all integer values there between. The time period may be at least 1, 2, 3, 4, 5, or 6 hours. The time period may be 10 to 24 hours. The incubation time period may be 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this disclosure. It may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. It may be desirable to enrich for or positively select for regulatory T cells which typically express CD4+, CD25+, CD62Lhi, GITR+, and FoxP3+. Alternatively, T regulatory cells can be depleted by anti-C25 conjugated beads or other similar method of selection.

A T cell population can be selected that expresses one or more of IFN-γ, TNF-alpha, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No. WO 2013/126712.

For isolation of a desired population of cells by positive or negative selection, the concentration of cells and surface (e.g., particles such as beads) can be varied. It may be desirable to significantly decrease the volume in which beads and cells are mixed together (e.g., increase the concentration of cells), to ensure maximum contact of cells and beads. For example, a concentration of 2 billion cells/mL may be used, or a concentration of 1 billion cells/mL is used. Greater than 100 million cells/mL may be used. A concentration of cells of 10, 15, 20, 25, 30, 35, 40, 45, or 50 million cells/mL may be used. A concentration of cells from 75, 80, 85, 90, 95, or 100 million cells/mL may be used. Concentrations of 125 or 150 million cells/mL can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

It may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. The concentration of cells used may be 5×10⁶/mL, or from about 1×10⁵/mL to 1×10⁶/mL, and any integer value in between. The cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1 per minute and stored in the vapor phase of a liquid nitrogen storage tank. Other methods of controlled freezing may be used as well as uncontrolled freezing immediately at −20° C. or in liquid nitrogen. Cryopreserved cells may be thawed and washed as described herein and allowed to rest for one hour at room temperature prior to activation using the methods of the present disclosure.

Also contemplated in the context of the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. A blood sample or an apheresis may be taken from a generally healthy subject. A blood sample or an apheresis is taken from a generally healthy subject who is at risk of developing a disease, but who has not yet developed a disease, and the cells of interest are isolated and frozen for later use. The T cells may be expanded, frozen, and used at a later time. Samples can be collected from a patient shortly after diagnosis of a particular disease as described herein but prior to any treatments. The cells can be isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and tacrolimus, antibodies, or other immunoablative agents such as alemtuzumab, anti-CD3 antibodies, cytoxan, fludarabine, cyclosporin, tacrolimus, rapamycin, mycophenolic acid, steroids, romidepsin, and irradiation.

T cells can be obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

### Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041, and 7,572,631.

Generally, the T cells of the present disclosure may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+T, CD8+ T cells or CD4+CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999). In some embodiments, T cells are activated by stimulation with an anti-CD3 antibody and an anti-CD28 antibody in combination with cytokines that bind the common gamma-chain (e.g., IL-2, IL-7, IL-12, IL-15, IL-21, and others). In some embodiments, T cells are activated by stimulation with an anti-CD3 antibody and an anti-CD28 antibody in combination with 10, 20, 30, 40, 50, 60, 70, 80, 90, 100, 200, 300, 400, 500, 600, 700, 800, 900, or 100 U/mL of IL-2, IL-7, and/or IL-15. In some embodiments, the cells are activated for 24 hours. In some embodiments, after transduction, the cells are expanded in the presence of anti-CD3 antibody, anti-CD28 antibody in combination with the same cytokines. In some embodiments, cells activated in the presence of activated by stimulation with an anti-CD3 antibody and an anti-CD28 antibody in combination with cytokines that bind the common gamma-chain are expanded in the presence of the same cytokines in the absence of the anti-CD3 antibody and anti-CD28 antibody after transduction. In some embodiments, cells are expanded for 4, 5, 6, 7, 8, 9, 10, 11, 12, 13, 14, 15, 16, 17, 18, 19, 20, 21, 22, 23, 24, 25, 26, 27, 28, 29, or 30 days.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once an anti-mesothelin TFP is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of an anti-mesothelin TFP are described in further detail below

Western blot analysis of TFP expression in primary T cells can be used to detect the presence of monomers and dimers (see, e.g., Milone et al., Molecular Therapy 17(8): 1453-1464 (2009)). Very briefly, T cells (1:1 mixture of CD4⁺ and CD8⁺ T cells) expressing the TFPs are expanded in vitro for more than 10 days followed by lysis and SDS-PAGE under reducing conditions. TFPs are detected by Western blotting using an antibody to a TCR chain. The same T cell subsets are used for SDS-PAGE analysis under non-reducing conditions to permit evaluation of covalent dimer formation.

In vitro expansion of TFP⁺ T cells following antigen stimulation can be measured by flow cytometry. For example, a mixture of CD4⁺ and CD8⁺ T cells are stimulated with alphaCD3/alphaCD28 and APCs followed by transduction with lentiviral vectors expressing GFP under the control of the promoters to be analyzed. Exemplary promoters include the CMV IE gene, EF-1alpha, ubiquitin C, or phosphoglycerokinase (PGK) promoters. GFP fluorescence is evaluated on day 6 of culture in the CD4+ and/or CD8+ T cell subsets by flow cytometry (see, e.g., Milone et al., Molecular Therapy 17(8): 1453-1464 (2009)). Alternatively, a mixture of CD4+ and CD8+ T cells are stimulated with alphaCD3/alphaCD28 coated magnetic beads on day 0, and transduced with TFP on day 1 using a bicistronic lentiviral vector expressing TFP along with eGFP using a 2A ribosomal skipping sequence. Cultures are re-stimulated with either mesothelin+K562 cells (K562-mesothelin), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/mL. GFP+ T cells are enumerated by flow cytometry using bead-based counting (see, e.g., Milone et al., Molecular Therapy 17(8): 1453-1464 (2009)).

Sustained TFP+ T cell expansion in the absence of re-stimulation can also be measured (see, e.g., Milone et al., Molecular Therapy 17(8): 1453-1464 (2009)). Briefly, mean T cell volume (fl) is measured on day 8 of culture using a Coulter Multisizer III particle counter following stimulation with alphaCD3/alphaCD28 coated magnetic beads on day 0, and transduction with the indicated TFP on day 1.

Animal models can also be used to measure a TFP-T activity. For example, xenograft model using human mesothelin-specific TFP+ T cells to treat a cancer in immunodeficient mice (see, e.g., Milone et al., Molecular Therapy 17(8): 1453-1464 (2009)). Very briefly, after establishment of cancer, mice are randomized as to treatment groups. Different numbers of engineered T cells are coinjected at a 1:1 ratio into NOD/SCID/γ−/− mice bearing cancer. The number of copies of each vector in spleen DNA from mice is evaluated at various times following T cell injection. Animals are assessed for cancer at weekly intervals. Peripheral blood mesothelin+ cancer cell counts are measured in mice that are injected with alphamesothelin-zeta TFP+ T cells or mock-transduced T cells. Survival curves for the groups are compared using the log-rank test. In addition, absolute peripheral blood CD4+ and CD8+ T cell counts 4 weeks following T cell injection in NOD/SCID/γ−/− mice can also be analyzed. Mice are injected with cancer cells and 3 weeks later are injected with T cells engineered to express TFP by a bicistronic lentiviral vector that encodes the TFP linked to eGFP. T cells are normalized to 45-50% input GFP+ T cells by mixing with mock-transduced cells prior to injection, and confirmed by flow cytometry. Animals are assessed for cancer at 1-week intervals. Survival curves for the TFP+ T cell groups are compared using the log-rank test.

Dose dependent TFP treatment response can be evaluated (see, e.g., Milone et al., Molecular Therapy 17(8): 1453-1464 (2009)). For example, peripheral blood is obtained 35-70 days after establishing cancer in mice injected on day 21 with TFP T cells, an equivalent number of mock-transduced T cells, or no T cells. Mice from each group are randomly bled for determination of peripheral blood mesothelin+ cancer cell counts and then killed on days 35 and 49. The remaining animals are evaluated on days 57 and 70.

Assessment of cell proliferation and cytokine production has been previously described, e.g., at Milone et al., Molecular Therapy 17(8): 1453-1464 (2009). Briefly, assessment of TFP-mediated proliferation is performed in microtiter plates by mixing washed T cells with cells expressing mesothelin or CD32 and CD137 (KT32-BBL) for a final T cell:cell expressing mesothelin ratio of 2:1. Cells expressing mesothelin cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T cell proliferation since these signals support long-term CD8+ T cell expansion ex vivo. T cells are enumerated in cultures using CountBright™ fluorescent beads (Invitrogen) and flow cytometry as described by the manufacturer. TFP+ T cells are identified by GFP expression using T cells that are engineered with eGFP-2A linked TFP-expressing lentiviral vectors. For TFP+ T cells not expressing GFP, the TFP+ T cells are detected with biotinylated recombinant mesothelin protein and a secondary avidin-PE conjugate. CD4+ and CD8+ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences) according the manufacturer's instructions. Fluorescence is assessed using a FACScalibur flow cytometer, and data is analyzed according to the manufacturer's instructions.

Cytotoxicity can be assessed by a standard ⁵¹Cr-release assay (see, e.g., Milone et al., Molecular Therapy 17(8): 1453-1464 (2009)). Briefly, target cells are loaded with ⁵¹Cr (as NaCrO₄, New England Nuclear) at 37° C. for 2 hours with frequent agitation, washed twice in complete RPMI medium and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released ⁵¹Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average ⁵¹Cr released for each experimental condition.

Imaging technologies can be used to evaluate specific trafficking and proliferation of TFPs in tumor-bearing animal models. Such assays have been described, e.g., in Barrett et al., Human Gene Therapy 22:1575-1586 (2011). Briefly, NOD/SCID/γc−/− (NSG) mice are injected IV with cancer cells followed 7 days later with T cells 4 hour after electroporation with the TFP constructs. The T cells are stably transfected with a lentiviral construct to express firefly luciferase, and mice are imaged for bioluminescence. Alternatively, therapeutic efficacy and specificity of a single injection of TFP+ T cells in a cancer xenograft model can be measured as follows: NSG mice are injected with cancer cells transduced to stably express firefly luciferase, followed by a single tail-vein injection of T cells electroporated with mesothelin TFP 7 days later. Animals are imaged at various time points post injection. For example, photon-density heat maps of firefly luciferase positive cancer in representative mice at day 5 (2 days before treatment) and day 8 (24 hours post TFP+ PBLs) can be generated.

Other assays, including those described in the Example section herein as well as those that are known in the art can also be used to evaluate the anti-mesothelin TFP constructs of the invention.

Mesothelin Associated Diseases and/or Disorders

In one aspect, the present disclosure provides methods for treating a disease associated with mesothelin expression. In one aspect, the present disclosure provides methods for treating a disease wherein part of the tumor is negative for mesothelin and part of the tumor is positive for mesothelin. For example, the TFP of the present disclosure is useful for treating subjects that have undergone treatment for a disease associated with elevated expression of mesothelin, wherein the subject that has undergone treatment for elevated levels of mesothelin exhibits a disease associated with elevated levels of mesothelin.

In one aspect, the present disclosure pertains to a method of inhibiting growth of a mesothelin-expressing tumor cell, comprising contacting the tumor cell with a mesothelin TFP T cell of the present invention such that the TFP-T is activated in response to the antigen and targets the cancer cell, wherein the growth of the tumor is inhibited.

In one aspect, the present disclosure pertains to a method of treating cancer in a subject. The method comprises administering to the subject a mesothelin TFP T cell of the present invention such that the cancer is treated in the subject. An example of a cancer that is treatable by the mesothelin TFP T cell of the present disclosure is a cancer associated with expression of mesothelin. In one aspect, the cancer is a mesothelioma. In one aspect, the cancer is selected from malignant pleural mesothelioma (MPM), non-small cell lung cancer (NSCLC), serous ovarian adenocarcinoma, or cholangiocarcinoma.

The present disclosure includes a type of cellular therapy where T cells are genetically modified to express a TFP and the TFP-expressing T cell is infused to a recipient in need thereof. The infused cell is able to kill tumor cells in the recipient. Unlike antibody therapies, TFP-expressing T cells are able to replicate in vivo, resulting in long-term persistence that can lead to sustained tumor control. In various aspects, the T cells administered to the patient, or their progeny, persist in the patient for at least one month, two month, three months, four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the T cell to the patient.

The present disclosure also includes a type of cellular therapy where T cells are modified, e.g., by in vitro transcribed RNA, to transiently express a TFP and the TFP-expressing T cell is infused to a recipient in need thereof. The infused cell is able to kill tumor cells in the recipient. Thus, in various aspects, the T cells administered to the patient, is present for less than one month, e.g., three weeks, two weeks, or one week, after administration of the T cell to the patient.

Without wishing to be bound by any particular theory, the anti-tumor immunity response elicited by the TFP-expressing T cells may be an active or a passive immune response, or alternatively may be due to a direct vs indirect immune response. The TFP transduced T cells may exhibit specific proinflammatory cytokine secretion and potent cytolytic activity in response to human cancer cells expressing the mesothelin antigen, resist soluble mesothelin inhibition, mediate bystander killing and/or mediate regression of an established human tumor. For example, antigen-less tumor cells within a heterogeneous field of mesothelin-expressing tumor may be susceptible to indirect destruction by mesothelin-redirected T cells that has previously reacted against adjacent antigen-positive cancer cells.

The human TFP-modified T cells of the present disclosure may be a type of vaccine for ex vivo immunization and/or in vivo therapy in a mammal, e.g., a human.

With respect to ex vivo immunization, at least one of the following occurs in vitro prior to administering the cell into a mammal: i) expansion of the cells, ii) introducing a nucleic acid encoding a TFP to the cells or iii) cryopreservation of the cells.

Ex vivo procedures are well known in the art and are discussed more fully herein. Briefly, cells are isolated from a mammal (e.g., a human) and genetically modified (i.e., transduced or transfected in vitro) with a vector expressing a TFP disclosed herein. The TFP-modified cell can be administered to a mammalian recipient to provide a therapeutic benefit. The mammalian recipient may be a human and the TFP-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present disclosure. Other suitable methods are known in the art, therefore the present disclosure is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the TFP-modified T cells of the present disclosure are used in the treatment of diseases, disorders and conditions associated with expression of mesothelin. The cells of the present disclosure may be used in the treatment of patients at risk for developing diseases, disorders and conditions associated with expression of mesothelin. Thus, the present disclosure provides methods for the treatment or prevention of diseases, disorders and conditions associated with expression of mesothelin comprising administering to a subject in need thereof, a therapeutically effective amount of the TFP-modified T cells of the disclosure.

The TFP-T cells of the present disclosure may be used to treat a proliferative disease such as a cancer or malignancy or a precancerous condition. In one aspect, he cancer is a mesothelioma. In one aspect, the cancer is selected from malignant pleural mesothelioma (MPM), non-small cell lung cancer (NSCLC), serous ovarian adenocarcinoma, or cholangiocarcinoma. Further, a disease associated with mesothelin expression includes, but is not limited to, e.g., atypical and/or non-classical cancers, malignancies, precancerous conditions or proliferative diseases expressing mesothelin. Non-cancer related indications associated with expression of mesothelin include, but are not limited to, e.g., autoimmune disease, (e.g., lupus), inflammatory disorders (allergy and asthma) and transplantation.

The TFP-modified T cells of the present disclosure may be administered either alone, or as a pharmaceutical composition in combination with diluents and/or with other components such as IL-2 or other cytokines or cell populations.

The present disclosure also provides methods for inhibiting the proliferation or reducing a mesothelin-expressing cell population, the methods comprising contacting a population of cells comprising a mesothelin-expressing cell with an anti-mesothelin TFP-T cell of the present disclosure that binds to the mesothelin-expressing cell. The anti-mesothelin TFP-T cell of the present disclosure may reduce the quantity, number, amount or percentage of cells and/or cancer cells by at least 25%, at least 30%, at least 40%, at least 50%, at least 65%, at least 75%, at least 85%, at least 95%, or at least 99% in a subject with or animal model a cancer associated with mesothelin-expressing cells relative to a negative control. In one aspect, the subject is a human.

The present disclosure also provides methods for preventing, treating and/or managing a disease associated with mesothelin-expressing cells (e.g., a cancer expressing mesothelin), the methods comprising administering to a subject in need an anti-mesothelin TFP-T cell of the present disclosure that binds to the mesothelin-expressing cell. In one aspect, the subject is a human. Non-limiting examples of disorders associated with mesothelin-expressing cells include autoimmune disorders (such as lupus), inflammatory disorders (such as allergies and asthma) and cancers (such as pancreatic cancer, ovarian cancer, stomach cancer, lung cancer, or endometrial cancer. or atypical cancers expressing mesothelin).

The present disclosure also provides methods for preventing, treating and/or managing a disease associated with mesothelin-expressing cells, the methods comprising administering to a subject in need an anti-mesothelin TFP-T cell of the present disclosure that binds to the mesothelin-expressing cell. In one aspect, the subject is a human.

The present disclosure provides methods for preventing relapse of cancer associated with mesothelin-expressing cells, the methods comprising administering to a subject in need thereof an anti-mesothelin TFP-T cell of the present disclosure that binds to the mesothelin-expressing cell. In one aspect, the methods comprise administering to the subject in need thereof an effective amount of an anti-mesothelin TFP-T cell described herein that binds to the mesothelin-bmca expressing cell in combination with an effective amount of another therapy.

### Combination Therapies

In some embodiments, the TFP T cells provided herein are administered with at least one additional therapeutic agent. Any suitable additional therapeutic agent may be administered with a TFP T cell provided herein. In some aspects, the additional therapeutic agent is selected from radiation, a cytotoxic agent, a chemotherapeutic agent, a cytostatic agent, an anti-hormonal agent, an EGFR inhibitor, an immunostimulatory agent, an anti-angiogenic agent, and combinations thereof.

In some embodiments, the additional therapeutic agent comprises an immunostimulatory agent.

In some embodiments, the immunostimulatory agent is an agent that blocks signaling of an inhibitory receptor of an immune cell, or a ligand thereof. In some aspects, the inhibitory receptor or ligand is selected from cytotoxic T-lymphocyte-associated protein 4 (CTLA-4, also known as CD152), programmed cell death protein 1 (also PD-1 or CD279), programmed death ligand 1 (also PD-L1 or CD274), transforming growth factor beta (TGFβ), lymphocyte-activation gene 3 (LAG-3, also CD223), Tim-3 (hepatitis A virus cellular receptor 2 or HAVCR2 or CD366), neuritin, B- and T-lymphocyte attenuator (also BTLA or CD272), killer cell immunoglobulin-like receptors (KIRs), and combinations thereof. In some aspects, the agent is selected from an anti-PD-1 antibody (e.g., pembrolizumab or nivolumab), and anti-PD-L1 antibody (e.g., atezolizumab), an anti-CTLA-4 antibody (e.g., ipilimumab), an anti-TIM3 antibody, carcinoembryonic antigen-related cell adhesion molecule 1 (CECAM-1, also CD66a) and 5 (CEACAM-5, also CD66e), vset immunoregulatory receptor (also VISR or VISTA), leukocyte-associated immunoglobulin-like receptor 1 (also LAIR1 or CD305), CD160, natural killer cell receptor 2B4 (also CD244 or SLAMF4), and combinations thereof. In some aspects, the agent is pembrolizumab. In some aspects, the agent is nivolumab. In some aspects, the agent is atezolizumab.

In some embodiments, the additional therapeutic agent is an agent that inhibits the interaction between PD-1 and PD-L1. In some aspects, the additional therapeutic agent that inhibits the interaction between PD-1 and PD-L1 is selected from an antibody, a peptidomimetic and a small molecule. In some aspects, the additional therapeutic agent that inhibits the interaction between PD-1 and PD-L1 is selected from pembrolizumab (KEYTRUDA), nivolumab (OPDIVO), atezolizumab, avelumab, pidilizumab, durvalumab, sulfamonomethoxine 1, and sulfamethizole 2. In some embodiments, the additional therapeutic agent that inhibits the interaction between PD-1 and PD-L1 is any therapeutic known in the art to have such activity, for example as described in Weinmann et al., Chem Med Chem, 2016, 14:1576 (DOI: 10.1002/cmdc.201500566), incorporated by reference in its entirety. In some embodiments, the agent that inhibits the interaction between PD-1 and PD-L1 is formulated in the same pharmaceutical composition an antibody provided herein. In some embodiments, the agent that inhibits the interaction between PD-1 and PD-L1 is formulated in a different pharmaceutical composition from an antibody provided herein. In some embodiments, the agent that inhibits the interaction between PD-1 and PD-L1 is administered prior to administration of an antibody provided herein. In some embodiments, the agent that inhibits the interaction between PD-1 and PD-L1 is administered after administration of an antibody provided herein. In some embodiments, the agent that inhibits the interaction between PD-1 and PD-L1 is administered contemporaneously with an antibody provided herein, but the agent and antibody are administered in separate pharmaceutical compositions.

In some embodiments, the immunostimulatory agent is an agonist of a co-stimulatory receptor of an immune cell. In some aspects, the co-stimulatory receptor is selected from GITR, OX40, ICOS, LAG-2, CD27, CD28, 4-1BB, CD40, STING, a toll-like receptor, RIG-1, and a NOD-like receptor. In some embodiments, the agonist is an antibody.

In some embodiments, the immunostimulatory agent modulates the activity of arginase, indoleamine-2 3-dioxygenase, or the adenosine A2A receptor.

In some embodiments, the immunostimulatory agent is a cytokine. In some aspects, the cytokine is selected from IL-2, IL-5, IL-7, IL-12, IL-15, IL-21, and combinations thereof.

In some embodiments, the immunostimulatory agent is an oncolytic virus. In some aspects, the oncolytic virus is selected from a herpes simplex virus, a vesicular stomatitis virus, an adenovirus, a Newcastle disease virus, a vaccinia virus, and a maraba virus.

Further examples of additional therapeutic agents include a taxane (e.g., paclitaxel or docetaxel); a platinum agent (e.g., carboplatin, oxaliplatin, and/or cisplatin); a topoisomerase inhibitor (e.g., irinotecan, topotecan, etoposide, and/or mitoxantrone); folinic acid (e.g., leucovorin); or a nucleoside metabolic inhibitor (e.g., fluorouracil, capecitabine, and/or gemcitabine). In some embodiments, the additional therapeutic agent is folinic acid, 5-fluorouracil, and/or oxaliplatin. In some embodiments, the additional therapeutic agent is 5-fluorouracil and irinotecan. In some embodiments, the additional therapeutic agent is a taxane and a platinum agent. In some embodiments, the additional therapeutic agent is paclitaxel and carboplatin. In some embodiments, the additional therapeutic agent is pemetrexate. In some embodiments, the additional therapeutic agent is a targeted therapeutic such as an EGFR, RAF or MEK-targeted agent.

The additional therapeutic agent may be administered by any suitable means. In some embodiments, a medicament provided herein, and the additional therapeutic agent are included in the same pharmaceutical composition. In some embodiments, an antibody provided herein, and the additional therapeutic agent are included in different pharmaceutical compositions.

In embodiments where an antibody provided herein and the additional therapeutic agent are included in different pharmaceutical compositions, administration of the antibody can occur prior to, simultaneously, and/or following, administration of the additional therapeutic agent. In some aspects, administration of an antibody provided herein, and the additional therapeutic agent occur within about one month of each other. In some aspects, administration of an antibody provided herein, and the additional therapeutic agent occur within about one week of each other. In some aspects, administration of an antibody provided herein, and the additional therapeutic agent occur within about one day of each other. In some aspects, administration of an antibody provided herein, and the additional therapeutic agent occur within about twelve hours of each other. In some aspects, administration of an antibody provided herein, and the additional therapeutic agent occur within about one hour of each other.

### Tumor Antigen Associated Diseases or Disorders

Many patients treated with cancer therapeutics that are directed to one target on a tumor cell, e.g., BCMA, CD19, CD20, CD22, CD123, MUC16, MSLN, etc., become resistant over time as escape mechanisms such as alternate signaling pathways and feedback loops become activated. Dual specificity therapeutics attempt to address this by combining targets that often substitute for each other as escape routes. Therapeutic T cell populations having TCRs specific to more than one tumor-associated antigen are promising combination therapeutics. In some embodiments, the dual specificity TFP T cells are administered with an additional anti-cancer agent; in some embodiments, the anti-cancer agent is an antibody or fragment thereof, another TFP T cell, a CAR T cell, or a small molecule. Exemplary tumor-associated antigens include, but are not limited to, oncofetal antigens (e.g., those expressed in fetal tissues and in cancerous somatic cells), oncoviral antigens (e.g., those encoded by tumorigenic transforming viruses), overexpressed/accumulated antigens (e.g., those expressed by both normal and neoplastic tissue, with the level of expression highly elevated in neoplasia), cancer-testis antigens (e.g., those expressed only by cancer cells and adult reproductive tissues such as testis and placenta), lineage-restricted antigens (e.g., those expressed largely by a single cancer histotype), mutated antigens (e.g., those expressed by cancer as a result of genetic mutation or alteration in transcription), posttranslationally altered antigens (e.g., those tumor-associated alterations in glycosylation, etc.), and idiotypic antigens (e.g., those from highly polymorphic genes where a tumor cell expresses a specific clonotype, e.g., as in B cell, T cell lymphoma/leukemia resulting from clonal aberrancies). Exemplary tumor-associated antigens include, but are not limited to, antigens of alpha-actinin-4, ARTC1, alphafetoprotein (AFP), BCR-ABL fusion protein (b3a2), B-RAF, CASP-5, CASP-8, beta-catenin, Cdc27, CDK4, CDK12, CDKN2A, CLPP, COA-1, CSNK1A1, CD79, CD79B, dek-can fusion protein, EFTUD2, Elongation factor 2, ETV6-AML1 fusion protein, FLT3-ITD, FNDC3B, FN1, GAS7, GPNMB, HAUS3, HSDL1, LDLR-fucosyltransferase AS fusion protein, HLA-A2d, HLA-A11d, hsp70-2, MART2, MATN, ME1, MUM-1f, MUM-2, MUM-3, neo-PAP, Myosin class I, NFYC, OGT, OS-9, p53, pml-RARalpha fusion protein, PPP1R3B, PRDX5, PTPRK, K-ras, N-ras, RBAF600, SIRT2, SNRPD1, SYT-SSX1 or -SSX2 fusion protein, TGF-betaRII, triosephosphate isomerase, BAGE-1, D393-CD20n, Cyclin-AL GAGE-1, GAGE-2, GAGE-8, GAGE-3, GAGE-4, GAGE-5, GAGE-6, GAGE-7, GnTVf, HERV-K-MEL, KK-LC-1, KM-HN-1, LAGE-1, LY6K, MAGE-A1, MAGE-A2, MAGE-A3, MAGE-A4, MAGE-A6, MAGE-A9, MAGE-A10, MAGE-A12 m, MAGE-C1, MAGE-C2, mucink, NA88-A, NY-ESO-1/LAGE-2, SAGE, Sp17, SSX-2, SSX-4, TAG-1, TAG-2, TRAG-3, TRP2-INT2g, XAGE-1b/GAGED2a, Gene/protein, CEA, gp100/Pme117, mammaglobin-A, Melan-A/MART-1, NY-BR-1, OAL PAP, PSA, RAB38/NY-MEL-1, TRP-1/gp75, TRP-2, tyrosinase, adipophilin, AIM-2, ALDH1A1, BCLX (L), BING-4, CALCA, CD45, CD274, CPSF, cyclin D1, DKK1, ENAH (hMena), EpCAM, EphA3, EZH2, FGF5, glypican-3, G250/MN/CAIX, HER-2/neu, HLA-DOB, Hepsin, IDOL IGF2B3, IL13Ralpha2, Intestinal carboxyl esterase, alpha-foetoprotein, Kallikrein 4, KIF20A, Lengsin, M-CSF, MCSP, mdm-2, Meloe, Midkine, MMP-2, MMP-7, MUC1, MUC5AC, p53, PAX5, PBF, PRAME, PSMA, RAGE-1, RGS5, RhoC, RNF43, RU2AS, secernin 1, SOX10, STEAP1, survivin, Telomerase, TPBG, VEGF, and WT1.

A TFP-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. The delivery of one treatment can still be occurring when the delivery of the second begins, so that there is overlap in terms of administration. This is sometimes referred to herein as “simultaneous” or “concurrent delivery”. Alternatively, the delivery of one treatment may end before the delivery of the other treatment begins. In either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment or the analogous situation is seen with the first treatment. Delivery can be such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The “at least one additional therapeutic agent” may include a TFP-expressing cell. Also provided are T cells that express multiple TFPs, which bind to the same or different target antigens, or same or different epitopes on the same target antigen. Also provided are populations of T cells in which a first subset of T cells express a first TFP and a second subset of T cells express a second TFP.

A TFP-expressing cell described herein and the at least one additional therapeutic agent can be administered simultaneously, in the same or in separate compositions, or sequentially. For sequential administration, the TFP-expressing cell described herein can be administered first, and the additional agent can be administered second, or the order of administration can be reversed.

A TFP-expressing cell described herein may be used in a treatment regimen in combination with surgery, chemotherapy, radiation, immunosuppressive agents, such as cyclosporin, azathioprine, methotrexate, mycophenolate, and tacrolimus, antibodies, or other immunoablative agents such as alemtuzumab, anti-CD3 antibodies or other antibody therapies, cytoxin, fludarabine, cyclosporin, tacrolimus, rapamycin, mycophenolic acid, steroids, romidepsin, cytokines, and irradiation. A TFP-expressing cell described herein may also be used in combination with a peptide vaccine, such as that described in Izumoto et al. 2008 J Neurosurg 108:963-971. A TFP-expressing cell described herein may also be used in combination with a promoter of myeloid cell differentiation (e.g., all-trans retinoic acid), an inhibitor of myeloid-derived suppressor cell (MDSC) expansion (e.g., inhibitors of c-kit receptor or a VEGF inhibitor), an inhibitor of MDSC function (e.g., COX2 inhibitors or phosphodiesterase-5 inhibitors), or therapeutic elimination of MDSCs (e.g., with a chemotherapeutic regimen such as treatment with doxorubicin and cyclophosphamide). Other therapeutic agents that may prevent the expansion of MDSCs include amino-biphosphonate, biphosphanate, sildenafil and tadalafil, nitroaspirin, vitamin D3, and gemcitabine. (See, e.g., Gabrilovich and Nagaraj, Nat. Rev. Immunol, (2009) v9(3): 162-174).

The subject can be administered an agent which reduces or ameliorates a side effect associated with the administration of a TFP-expressing cell. Side effects associated with the administration of a TFP-expressing cell include, but are not limited to cytokine release syndrome (CRS), and hemophagocytic lymphohistiocytosis (HLH), also termed Macrophage Activation Syndrome (MAS). Symptoms of CRS include high fevers, nausea, transient hypotension, hypoxia, and the like. Accordingly, the methods described herein can comprise administering a TFP-expressing cell described herein to a subject and further administering an agent to manage elevated levels of a soluble factor resulting from treatment with a TFP-expressing cell. The soluble factor elevated in the subject is one or more of IFN-γ, TNFα, IL-2, IL-6 and IL8. Therefore, an agent administered to treat this side effect can be an agent that neutralizes one or more of these soluble factors. Such agents include, but are not limited to a steroid, an inhibitor of TNFα, and an inhibitor of IL-6. An example of a TNFα inhibitor is entanercept. An example of an IL-6 inhibitor is tocilizumab (toc).

The subject can be administered an agent which enhances the activity of a TFP-expressing cell. For example, the agent can be an agent which inhibits an inhibitory molecule. Inhibitory molecules, e.g., Programmed Death 1 (PD1), can, decrease the ability of a TFP-expressing cell to mount an immune effector response. Examples of inhibitory molecules include PD1, PD-L1, CTLA4, TIM3, LAG3, VISTA, BTLA, TIGIT, LAIR1, CD160, 2B4 and TGFR beta. Inhibition of an inhibitory molecule, e.g., by inhibition at the DNA, RNA or protein level, can optimize a TFP-expressing cell performance. An inhibitory nucleic acid, e.g., an inhibitory nucleic acid, e.g., a dsRNA, e.g., an siRNA or shRNA, can be used to inhibit expression of an inhibitory molecule in the TFP-expressing cell. The inhibitor can be a shRNA. The inhibitory molecule is inhibited within a TFP-expressing cell. In these cases, a dsRNA molecule that inhibits expression of the inhibitory molecule is linked to the nucleic acid that encodes a component, e.g., all of the components, of the TFP. The inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy™; Bristol-Myers Squibb; tremelimumab (IgG2 monoclonal antibody available from Pfizer, formerly known as ticilimumab, CP-675,206)). The agent is an antibody or antibody fragment that binds to TIM3. The agent is an antibody or antibody fragment that binds to LAG3.

The T cells may be altered (e.g., by gene transfer) in vivo via a lentivirus, e.g., a lentivirus specifically targeting a CD4+ or CD8+ T cell. (See, e.g., Zhou et al., J. Immunol. (2015) 195:2493-2501).

The agent which enhances the activity of a TFP-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comprising an intracellular signaling domain as described herein. The polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. The fusion protein can be expressed by the same cell that expressed the TFP. The fusion protein may be expressed by a cell, e.g., a T cell that does not express an anti-mesothelin TFP.

The human or humanized antibody domain comprising an antigen binding domain that is an anti-mesothelin binding domain encoded by the nucleic acid, or an antibody comprising the anti-mesothelin binding domain, or a cell expressing the anti-mesothelin binding domain encoded by the nucleic acid can have an affinity value of at most about 200 nM, 100 nM, 75 nM, a 50 nM, 25 nM, 20 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, or 0.01 nM; and/or at least about 100 nM, 75 nM, a 50 nM, 25 nM, 20 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, or 0.01 nM; and or about 200 nM, 100 nM, 75 nM, a 50 nM, 25 nM, 20 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, 0.1 nM, 0.09 nM, 0.08 nM, 0.07 nM, 0.06 nM, 0.05 nM, 0.04 nM, 0.03 nM, 0.02 nM, or 0.01 nM.

### Pharmaceutical Compositions

Pharmaceutical compositions of the present disclosure may comprise a TFP-expressing cell, e.g., a plurality of TFP-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

The pharmaceutical composition can be substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. The bacterium may be at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

It may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. T cells can be activated from blood draws of from 10 cc to 400 cc. T cells can be activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. The T cell compositions of the present disclosure can be administered to a patient by intradermal or subcutaneous injection. The T cell compositions of the present disclosure can be administered by i.v. injection. The compositions of T cells may be injected directly into a tumor, lymph node, or site of infection. In some embodiments, described herein are compositions for parenteral administration which comprises a solution of cells is dissolved or suspended in an acceptable carrier, for example, an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The compositions can contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

In a particular exemplary aspect, subjects may undergo leukapheresis, wherein leukocytes are collected, enriched, or depleted ex vivo to select and/or isolate the cells of interest, e.g., T cells. These T cell isolates may be expanded by methods known in the art and treated such that one or more TFP constructs of the present disclosure may be introduced, thereby creating a TFP-expressing T cell of the disclosure. Subjects in need thereof may subsequently undergo standard treatment with high dose chemotherapy followed by peripheral blood stem cell transplantation. Following or concurrent with the transplant, subjects may receive an infusion of the expanded TFP T cells of the present disclosure. Expanded cells may be administered before or following surgery.

The dosage of the above treatments to be administered to a patient will vary with the precise nature of the condition being treated and the recipient of the treatment. The scaling of dosages for human administration can be performed according to art-accepted practices. The dose for alemtuzumab, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

The TFP can be introduced into T cells, e.g., using in vitro transcription, and the subject (e.g., human) receives an initial administration of TFP T cells of the disclosure, and one or more subsequent administrations of the TFP T cells of the disclosure, wherein the one or more subsequent administrations are administered less than 15 days, e.g., 14, 13, 12, 11, 10, 9, 8, 7, 6, 5, 4, 3, or 2 days after the previous administration. More than one administration of the TFP T cells of the present disclosure may be administered to the subject (e.g., human) per week, e.g., 2, 3, or 4 administrations of the TFP T cells of the present disclosure are administered per week. The subject (e.g., human subject) may receive more than one administration of the TFP T cells per week (e.g., 2, 3 or 4 administrations per week) (also referred to herein as a cycle), followed by a week of no TFP T cells administrations, and then one or more additional administration of the TFP T cells (e.g., more than one administration of the TFP T cells per week) is administered to the subject. The subject (e.g., human subject) may receive more than one cycle of TFP T cells, and the time between each cycle is less than 10, 9, 8, 7, 6, 5, 4, or 3 days. The TFP T cells can be administered every other day for 3 administrations per week. The TFP T cells of the present disclosure can be administered for at least two, three, four, five, six, seven, eight or more weeks.

Mesothelin TFP T cells can be generated using lentiviral viral vectors, such as lentivirus. TFP-T cells generated that way will have stable TFP expression.

TFP T cells may transiently express TFP vectors for 4, 5, 6, 7, 8, 9, 10, 11, 12, 13, 14, 15 days after transduction. Transient expression of TFPs can be effected by RNA TFP vector delivery. The TFP RNA can be transduced into the T cell by electroporation.

A potential issue that can arise in patients being treated using transiently expressing TFP T cells (particularly with murine scFv bearing TFP T cells) is anaphylaxis after multiple treatments.

Without being bound by this theory, it is believed that such an anaphylactic response might be caused by a patient developing humoral anti-TFP response, i.e., anti-TFP antibodies having an anti-IgE isotype. It is thought that a patient's antibody producing cells undergo a class switch from IgG isotype (that does not cause anaphylaxis) to IgE isotype when there is a ten to fourteen-day break in exposure to antigen.

If a patient is at high risk of generating an anti-TFP antibody response during the course of transient TFP therapy (such as those generated by RNA transductions), TFP T cell infusion breaks should not last more than ten to fourteen days.

### Methods of Treatment

Also provided herein are methods of treating a subject with a disease, disorder or condition. A method of treatment can comprise administering a pharmaceutical composition disclosed herein to a subject with a disease, disorder or condition. The present disclosure provides methods of treatment comprising an immunogenic therapy. Methods of treatment for a disease (such as cancer or a viral infection) are provided. A method can comprise administering to a subject an effective amount of a pharmaceutical composition comprising anti-MSLN TFP T cells.

In some embodiments, the method of treating a subject with a disease or condition comprises administering to the subject the pharmaceutical composition disclosed herein. In some embodiments, the method is a method of preventing resistance to a cancer therapy, wherein the method comprises administering to a subject in need thereof the pharmaceutical composition disclosed herein. In some embodiments, the method is a method of inducing an immune response, wherein the method comprises administering to a subject in need thereof the pharmaceutical composition disclosed herein. In some embodiments, the immune response is a humoral response. In some embodiments, the immune response is a cytotoxic T cell response.

In some embodiments, the subject has cancer, wherein the cancer is selected from the group consisting of mesothelioma, ovarian cancer, cholangiocarcinoma, lung adenocarcinoma, triple-negative breast cancer, and pancreatic adenocarcinoma.

In some embodiments, the method further comprises administering at least one additional therapeutic agent or modality. In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering the pharmaceutical composition disclosed herein.

In some embodiments, the additional therapeutic agent is administered before, simultaneously, or after administering the pharmaceutical composition disclosed herein.

In some embodiments, the cancer is selected from the group consisting of carcinoma, lymphoma, blastoma, sarcoma, leukemia, squamous cell cancer, lung cancer (including small cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, melanoma, endometrial or uterine carcinoma, salivary gland carcinoma, kidney or renal cancer, liver cancer, prostate cancer, vulval cancer, thyroid cancer, hepatic carcinoma, head and neck cancer, colorectal cancer, rectal cancer, soft-tissue sarcoma, Kaposi's sarcoma, B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's macroglobulinemia), chronic lymphocytic leukemia (CLL), acute lymphoblastic leukemia (ALL), myeloma, Hairy cell leukemia, chronic myeloblasts leukemia, and post-transplant lymphoproliferative disorder (PTLD), abnormal vascular proliferation associated with phakomatoses, edema, Meigs' syndrome, and combinations thereof.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NEIL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, malignant pleural mesothelioma (MPM), non-small cell lung cancer (NSCLC), serous ovarian adenocarcinoma, or cholangiocarcinoma.

The pharmaceutical compositions provided herein may be used alone or in combination with conventional therapeutic regimens such as surgery, irradiation, chemotherapy and/or bone marrow transplantation (autologous, syngeneic, allogeneic or unrelated).

In some embodiments, at least one or more chemotherapeutic agents may be administered in addition to the pharmaceutical composition comprising an immunogenic therapy. In some embodiments, the one or more chemotherapeutic agents may belong to different classes of chemotherapeutic agents.

In practicing the methods of treatment or use provided herein, therapeutically-effective amounts of the pharmaceutical compositions can be administered to a subject having a disease or condition. A therapeutically-effective amount can vary widely depending on the severity of the disease, the age and relative health of the subject, the potency of the compounds used, and other factors.

In some embodiments, the methods of treatment include one or more rounds of leukapheresis prior to transplantation of T cells. The leukapheresis may include collection of peripheral blood mononuclear cells (PBMCs). Leukapheresis may include mobilizing the PBMCs prior to collection. Alternatively, non-mobilized PBMCs may be collected. A large volume of PBMCs may be collected from the subject in one round. Alternatively, the subject may undergo two or more rounds of leukapheresis. The volume of apheresis may be dependent on the number of cells required for transplant. For instance, 12-15 litres of non-mobilized PBMCs may be collected from a subject in one round. The number of PBMCs to be collected from a subject may be between 1×10⁸ to 5×10¹⁰ cells. The number of PBMCs to be collected from a subject may be 1×10⁸, 5×10⁸, 1×10⁹, 5×10⁹, 1×10¹⁰ or 5×10¹⁰ cells. The minimum number of PBMCs to be collected from a subject may be 1×10⁶/kg of the subject's weight. The minimum number of PBMCs to be collected from a subject may be 1×10⁶/kg, 5×10⁶/kg, 1×10⁷/kg, 5×10⁷/kg, 1×10⁸/kg, 5×10⁸/kg of the subject's weight.

In some embodiments, the methods of treatment include cancer treatment of a subject prior to administering anti-MSLN TFP cells. The cancer treatment may include chemotherapy, immunotherapy, targeted agents, radiation and high dose corticosteroid. The methods may include administering chemotherapy to a subject including lymphodepleting chemotherapy using high doses of myeloablative agents. In some embodiments, the methods include administering a preconditioning agent, such as a lymphodepleting or chemotherapeutic agent, such as cyclophosphamide, fludarabine, or combinations thereof, to a subject prior to the first or subsequent dose. For example, the subject may be administered a preconditioning agent at least 2 days prior, such as at least 3, 4, 5, 6, 7, 8, 9 or 10 days prior, to the first or subsequent dose. In some embodiments, the subject is administered a preconditioning agent no more than 10 days prior, such as no more than 9, 8, 7, 6, 5, 4, 3, or 2 days prior, to the first or subsequent dose.

In some embodiments, where the lymphodepleting agent comprises cyclophosphamide, the subject is administered between 0.3 grams per square meter of the body surface of the subject (g/m²) and 5 g/m² cyclophosphamide. In some cases, the amount of cyclophosphamide administered to a subject is about at least 0.3 g/m². In some cases, the amount of cyclophosphamide administered to a subject is about at most 5 g/m². In some cases, the amount of cyclophosphamide administered to a subject is about 0.3 g/m² to 0.4 g/m², 0.3 g/m² to 0.5 g/m², 0.3 g/m² to 0.6 g/m², 0.3 g/m² to 0.7 g/m², 0.3 g/m² to 0.8 g/m², 0.3 g/m² to 0.9 g/m², 0.3 g/m² to 1 g/m², 0.3 g/m² to 2 g/m², 0.3 g/m² to 3 g/m², 0.3 g/m² to 4 g/m², 0.3 g/m² to 5 g/m², 0.4 g/m² to 0.5 g/m², 0.4 g/m² to 0.6 g/m², 0.4 g/m² to 0.7 g/m², 0.4 g/m² to 0.8 g/m², 0.4 g/m² to 0.9 g/m², 0.4 g/m² to 1 g/m², 0.4 g/m² to 2 g/m², 0.4 g/m² to 3 g/m², 0.4 g/m² to 4 g/m², 0.4 g/m² to 5 g/m², 0.5 g/m² to 0.6 g/m², 0.5 g/m² to 0.7 g/m², 0.5 g/m² to 0.8 g/m², 0.5 g/m² to 0.9 g/m², 0.5 g/m² to 1 g/m², 0.5 g/m² to 2 g/m², 0.5 g/m² to 3 g/m², 0.5 g/m² to 4 g/m², 0.5 g/m² to 5 g/m², 0.6 g/m² to 0.7 g/m², 0.6 g/m² to 0.8 g/m², 0.6 g/m² to 0.9 g/m², 0.6 g/m² to 1 g/m², 0.6 g/m² to 2 g/m², 0.6 g/m² to 3 g/m², 0.6 g/m² to 4 g/m², 0.6 g/m² to 5 g/m², 0.7 g/m² to 0.8 g/m², 0.7 g/m² to 0.9 g/m², 0.7 g/m² to 1 g/m², 0.7 g/m² to 2 g/m², 0.7 g/m² to 3 g/m², 0.7 g/m² to 4 g/m², 0.7 g/m² to 5 g/m², 0.8 g/m² to 0.9 g/m², 0.8 g/m² to 1 g/m², 0.8 g/m² to 2 g/m², 0.8 g/m² to 3 g/m², 0.8 g/m² to 4 g/m², 0.8 g/m² to 5 g/m², 0.9 g/m² to 1 g/m², 0.9 g/m² to 2 g/m², 0.9 g/m² to 3 g/m², 0.9 g/m² to 4 g/m², 0.9 g/m² to 5 g/m², 1 g/m² to 2 g/m², 1 g/m² to 3 g/m², 1 g/m² to 4 g/m², 1 g/m² to 5 g/m², 2 g/m² to 3 g/m², 2 g/m² to 4 g/m², 2 g/m² to 5 g/m², 3 g/m² to 4 g/m², 3 g/m² to 5 g/m², or 4 g/m² to 5 g/m². In some cases, the amount of cyclophosphamide administered to a subject is about 0.3 g/m², 0.4 g/m², 0.5 g/m², 0.6 g/m², 0.7 g/m², 0.8 g/m², 0.9 g/m², 1 g/m², 2 g/m², 3 g/m², 4 g/m², or 5 g/m². In some embodiments, the subject is preconditioned with cyclophosphamide at a dose between or between about 200 mg/kg and 1000 mg/kg, such as between or between about 400 mg/kg and 800 mg/kg. In some aspects, the subject is preconditioned with or with about 600 mg/kg of cyclophosphamide. In some embodiments, the cyclophosphamide can be administered in a single dose or can be administered in a plurality of doses, such as given daily, every other day or every three days. For example, in some instances, the agent, e.g., cyclophosphamide, is administered between or between about 1 and 5 times, such as between or between about 2 and 4 times. In some embodiments, such plurality of doses is daily, such as on days −6 through −4 relative to administration of anti-MSLN TFP T cells.

In some embodiments, where the lymphodepleting agent comprises fludarabine, the subject is administered fludarabine at a dose between or between about 1 milligrams per square meter of the body surface of the subject (mg/m²) and 100 mg/m². In some cases, the amount of fludarabine administered to a subject is about at least 1 mg/m². In some cases, the amount of fludarabine administered to a subject is about at most 100 mg/m². In some cases, the amount of fludarabine administered to a subject is about 1 mg/m² to 5 mg/m², 1 mg/m² to 10 mg/m², 1 mg/m² to 15 mg/m², 1 mg/m² to 20 mg/m², 1 mg/m² to 30 mg/m², 1 mg/m² to 40 mg/m², 1 mg/m² to 50 mg/m², 1 mg/m² to 70 mg/m², 1 mg/m² to 90 mg/m², 1 mg/m² to 100 mg/m², 5 mg/m² to 10 mg/m², 5 mg/m² to 15 mg/m², 5 mg/m² to 20 mg/m², 5 mg/m² to 30 mg/m², 5 mg/m² to 40 mg/m², 5 mg/m² to 50 mg/m², 5 mg/m² to 70 mg/m², 5 mg/m² to 90 mg/m², 5 mg/m² to 100 mg/m², 10 mg/m² to 15 mg/m², 10 mg/m² to 20 mg/m², 10 mg/m² to 30 mg/m², 10 mg/m² to 40 mg/m², 10 mg/m² to 50 mg/m², 10 mg/m² to 70 mg/m², 10 mg/m² to 90 mg/m², 10 mg/m² to 100 mg/m², 15 mg/m² to 20 mg/m², 15 mg/m² to 30 mg/m², 15 mg/m² to 40 mg/m², 15 mg/m² to 50 mg/m², 15 mg/m² to 70 mg/m², 15 mg/m² to 90 mg/m², 15 mg/m² to 100 mg/m², 20 mg/m² to 30 mg/m², 20 mg/m² to 40 mg/m², 20 mg/m² to 50 mg/m², 20 mg/m² to 70 mg/m², 20 mg/m² to 90 mg/m², 20 mg/m² to 100 mg/m², 30 mg/m² to 40 mg/m², 30 mg/m² to 50 mg/m², 30 mg/m² to 70 mg/m², 30 mg/m² to 90 mg/m², 30 mg/m² to 100 mg/m², 40 mg/m² to 50 mg/m², 40 mg/m² to 70 mg/m², 40 mg/m² to 90 mg/m², 40 mg/m² to 100 mg/m², 50 mg/m² to 70 mg/m², 50 mg/m² to 90 mg/m², 50 mg/m² to 100 mg/m², 70 mg/m² to 90 mg/m², 70 mg/m² to 100 mg/m², or 90 mg/m² to 100 mg/m². In some cases, the amount of fludarabine administered to a subject is about 1 mg/m², 5 mg/m², 10 mg/m², 15 mg/m², 20 mg/m², 30 mg/m², 40 mg/m², 50 mg/m², 70 mg/m², 90 mg/m², or 100 mg/m². In some embodiments, the fludarabine can be administered in a single dose or can be administered in a plurality of doses, such as given daily, every other day or every three days. For example, in some instances, the agent, e.g., fludarabine, is administered between or between about 1 and 5 times, such as between or between about 3 and 5 times. In some embodiments, such plurality of doses is administered daily, such as on days −7 through −4 relative to administration of anti-MSLN TFP T cells.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 400 mg/m² of cyclophosphamide and one or more doses of 20 mg/m² fludarabine prior to the first or subsequent dose of T cells. In some examples, the subject is administered 500 mg/m² of cyclophosphamide and one or more doses of 25 mg/m² fludarabine prior to the first or subsequent dose of T cells. In some examples, the subject is administered 600 mg/m² of cyclophosphamide and one or more doses of 30 mg/m² fludarabine prior to the first or subsequent dose of T cells. In some examples, the subject is administered 700 mg/m² of cyclophosphamide and one or more doses of 35 mg/m² fludarabine prior to the first or subsequent dose of T cells. In some examples, the subject is administered 700 mg/m² of cyclophosphamide and one or more doses of 40 mg/m² fludarabine prior to the first or subsequent dose of T cells. In some examples, the subject is administered 800 mg/m² of cyclophosphamide and one or more doses of 45 mg/m² fludarabine prior to the first or subsequent dose of T cells.

Fludarabine and cyclophosphamide may be administered on alternative days. In some cases, fludarabine and cyclophosphamide may be administered concurrently. In some cases, an initial dose of fludarabine is followed by a dose of cyclophosphamide. In some cases, an initial dose of cyclophosphamide may be followed by an initial dose of fludarabine. In some examples, a treatment regimen may include treatment of a subject with an initial dose of fludarabine 10 days prior to the transplant, followed by treatment with an initial dose of cyclophosphamide administered 9 days prior to the cell transplant, concurrently with a second dose of fludarabine. In some examples, a treatment regimen may include treatment of a subject with an initial dose of fludarabine 8 days prior to the transplant, followed by treatment with an initial dose of cyclophosphamide administered 7 days prior to the transplant concurrently with a second dose of fludarabine.

The anti-MSLN TFP T cell product may be administered as one or more infusions. In some cases, a subject is administered one dose of T cells. In some cases, a subject is administered more than one doses of T cells. In some cases, a subject is administered three doses of T cells. In some cases, a subject is administered four doses of T cells. In some cases, a subject is administered five or more doses of T cells. In some embodiments, two consecutive doses of T cells are administered no less than 60 days and no more than 12 months apart. In some embodiments, two doses of T cells are administered no more than 60 days apart. In some embodiments, the more than one doses of T cells are evenly spaced. In some embodiments, the one or more doses of T cells are not evenly spaced.

A single infusion may comprise a dose between 1×10⁶ transduced cells per square meter body surface of the subject (cells/m²) and 5×10⁹ transduced cells/m². A single infusion may comprise between about 2.5×10⁶ to about 5×10⁹ transduced cells/m². A single infusion may comprise between at least about 2.5×10⁶ transduced cells/m². A single infusion may comprise between at most 5×10⁹ transduced cells/m². A single infusion may comprise between 1×10⁶ to 1×10⁸, 1×10⁶ to 2.5×10⁸, 1×10⁶ to 5×10⁸, 1×10⁶ to 1×10⁹, 1×10⁶ to 5×10⁹, 2.5×10⁶ to 5×10⁶, 2.5×10⁶ to 7.5×10⁶, 2.5×10⁶ to 1×10⁷, 2.5×10⁶ to 5×10⁷, 2.5×10⁶ to 7.5×10⁷, 2.5×10⁶ to 1×10⁸, 2.5×10⁶ to 2.5×10⁸, 2.5×10⁶ to 5×10⁸, 2.5×10⁶ to 1×10⁹, 2.5×10⁶ to 5×10⁹, 5×10⁶ to 7.5×10⁶, 5×10⁶ to 1×10⁷, 5×10⁶ to 5×10⁷, 5×10⁶ to 7.5×10⁷, 5×10⁶ to 1×10⁸, 5×10⁶ to 2.5×10⁸, 5×10⁶ to 5×10⁸, 5×10⁶ to 1×10⁹, 5×10⁶ to 5×10⁹, 7.5×10⁶ to 1×10⁷, 7.5×10⁶ to 5×10⁷, 7.5×10⁶ to 7.5×10⁷, 7.5×10⁶ to 1×10⁸, 7.5×10⁶ to 2.5×10⁸, 7.5×10⁶ to 5×10⁸, 7.5×10⁶ to 1×10⁹, 7.5×10⁶ to 5×10⁹, 1×10⁷ to 5×10⁷, 1×10⁷ to 7.5×10⁷, 1×10⁷ to 1×10⁸, 1×10⁷ to 2.5×10⁸, 1×10⁷ to 5×10⁸, 1×10⁷ to 1×10⁹, 1×10⁷ to 5×10⁹, 4.25×10⁷ to 7.5×10⁷, 5×10⁷ to 7.5×10⁷, 5×10⁷ to 1×10⁸, 5×10⁷ to 2.5×10⁸, 5×10⁷ to 5×10⁸, 5×10⁷ to 1×10⁹, 5×10⁷ to 5×10⁹, 7.5×10⁷ to 1×10⁸, 7.5×10⁷ to 2.5×10⁸, 7.5×10⁷ to 5×10⁸, 7.5×10⁷ to 1×10⁹, 7.5×10⁷ to 5×10⁹, 1×10⁸ to 2.5×10⁸, 1×10⁸ to 5×10⁸, 1×10⁸ to 1×10⁹, 1×10⁸ to 5×10⁹, 2.5×10⁸ to 5×10⁸, 2.5×10⁸ to 1×10⁹, 2.5×10⁸ to 5×10⁹, 5×10⁸ to 1×10⁹, 5×10⁸ to 5×10⁹, or 1×10⁹ to 5×10⁹ transduced cells/m². A single infusion may comprise between 1×10⁶ transduced cells/m², 2.5×10⁶ transduced cells/m², 5×10⁶ transduced cells/m², 7.5×10⁶ transduced cells/m², 1×10⁷ transduced cells/m², 4.25×10⁷ transduced cells/m², 5×10⁷ transduced cells/m², 7.5×10⁷ transduced cells/m², 1×10⁸ transduced cells/m², 2.5×10⁸ transduced cells/m², 5×10⁸ transduced cells/m², 1×10⁹ transduced cells/m², or 5×10⁹ transduced cells/m². In some embodiments, a subject is administered more than one dose of T cells and each dose has the same number of transduced T cells. In some embodiments, a subject is administered more than one dose of T cells and one or more of the doses do not have the same number of transduced T cells.

In one example, the method of treatment may comprise an initial PBMC collection from a subject. 1×10⁶ to 1×10⁸ PBMCs/kg of the subject weight may be collected. The PBMC fraction collected from the subject may then be enriched for T cells. Enriched T cells may be transduced as described herein to express anti-MSLN T cell receptor fusion protein (TFP). In some cases, the transduced T cells may be expanded and/or crysopreserved. The subject may undergo lymphodepletig chemotherapy following the leukapheresis. An alternating dose of fludarabine and cyclophosphamide may be administered to the subject. The dosing schedule may be one described elsewhere herein. In one example, the dose of fludarabine or an equivalent chemoitherapeutic agent administered to the subject may be between 15 mg/m² to 45 mg/m². The dose of cyclophosphamide or an equivalent chemotherapeutic agent administered to the subject may be between 400 g/m² to 800 mg/m². The doses of fludarabine and cyclophosphamide may be administered in an alternating manner, for instance in this scenario an initial dose of fludarabine may be followed by an initial dose of cyclophosphamide. The administration of lymphodepleting agents may be followed by the transplantation of anti-MSLN TFP producing T cells. T cells may be administered intravenously as a single dose to the subject. A single infusion of cells may comprise between 1×10⁷ transduced cells/m² to 5×10⁹ transduced cells/m².

In another example, the method of treatment may comprise an initial PBMC collection from a subject. 1×10⁶ to 1×10⁸ PBMCs/kg of the subject weight may be collected. The PBMC fraction collected from the subject may then be enriched for T cells. Enriched T cells may be transduced as described herein to express anti-MSLN T cell receptor fusion protein (TFP). An alternating dose of fludarabine and cyclophosphamide may be administered to the subject after the leukapheresis is complete. The dosing schedule may be one described elsewhere herein. In one example, the dose of fludarabine or an equivalent chemotherapeutic agent administered to the subject may be 20 mg/m². The dose of cyclophosphamide or an equivalent chemotherapeutic agent administered to the subject may be 700 g/m². The doses of fludarabine and cyclophosphamide may be administered in an alternating manner, for instance in this scenario an initial dose of fludarabine may be followed by an initial dose of cyclophosphamide. An initial dose of fludarabine may be administered on day −9 of the T cell transplant. Other doses of fludarabine may be administered on days −8, −7, −6, −5, −4 and −3. An initial dose of cyclophosphamide may be administered on day −7 concurrently with the fludarabine. Other doses of cyclophosphamide may be administered on days −5 and −4. The administration of lymphodepleting agents may be followed by the transplantation of anti-MSLN TFP producing T cells. T cells may be administered intravenously as a single dose to the subject. A single infusion of cells may comprise at least 1×10⁷ transduced cells/m². A single infusion of cells may comprise at most 1×10¹⁰ transduced cell s/m².

In another example, the method of treatment may comprise an initial PBMC collection from a subject. 1×10⁶ to 1×10⁸ PBMCs/kg of the subject weight may be collected. The PBMC fraction collected from the subject may then be enriched for T cells. Enriched T cells may be transduced as described herein to express anti-MSLN T cell receptor fusion protein (TFP). An alternating dose of fludarabine and cyclophosphamide may be administered to the subject after the leukapheresis is complete. The dosing schedule may be one described elsewhere herein. In one example, the dose of fludarabine or an equivalent chemoitherapeutic agent administered to the subject may be 40 mg/m². The dose of cyclophosphamide or an equivalent chemotherapeutic agent administered to the subject may be 400 g/m². The doses of fludarabine and cyclophosphamide may be administered in an alternating manner, for instance in this scenario an initial dose of fludarabine may be followed by an initial dose of cyclophosphamide. An initial dose of fludarabine may be administered on day −6 of the T cell transplant. Other doses of fludarabine may be administered on days −5 and −4. An initial dose of cyclophosphamide may be administered on day −5 concurrently with the fludarabine. Another dose of cyclophosphamide may be administered on day −3. The administration of lymphodepleting agents may be followed by the transplantation of anti-MSLN TFP producing T cells. T cells may be administered intravenously as a single dose to the subject. A single infusion of cells may comprise at least 1×10⁸ transduced cells/m². A single infusion of cells may comprise at most 1×10⁹ transduced cells/m².

In another example, the method of treatment may comprise an initial PBMC collection from a subject. 1×10⁶ to 1×10⁸ PBMCs/kg of the subject weight may be collected. The PBMC fraction collected from the subject may then be enriched for T cells. Enriched T cells may be transduced as described herein to express anti-MSLN T cell receptor fusion protein (TFP). In some cases, the transduced T cells may be expanded and/or crysopreserved. In this example, the subject does not undergo lymphodepletig chemotherapy following the leukapheresis. Anti-MSLN TFP producing T cells may be administered intravenously as a single dose to the subject. A single infusion of cells may comprise between 1×10⁸ cells/m² to 10×10⁹ cells/m².

### EXEMPLARY EMBODIMENTS

Embodiment 1: A method for the treatment of a human patient diagnosed with a mesothelin (MSLN)-expressing unresectable, metastatic, or recurrent cancer, the method comprising administering to the patient a first dose comprising an amount of transduced anti-MSLN T cell receptor fusion protein (TFP) T cells, and further comprising administering one or more additional doses, wherein the first dose and each additional doses comprise from about 5×107 to about 1×109 transduced cells/m2.

Embodiment 2: The method of embodiment 1, wherein the cancer comprises malignant pleural mesothelioma (MPM), non-small cell lung cancer (NSCLC), serous ovarian adenocarcinoma, or cholangiocarcinoma.

Embodiment 3: The method of embodiment 1, comprising administering one, two, three, or more than three additional doses of anti-MSLN TFP T cells in evenly spaced increments.

Embodiment 4: The method of embodiment 1, comprising administering four doses of anti-MSLN TFP T cells in evenly spaced increments, comprising a first dose, a second dose, a third dose, and a fourth dose.

Embodiment 5: The method of embodiment 1, wherein the anti-MSLN TFP T cells is administered via intravenous infusion.

Embodiment 6: The method of embodiment 1, wherein the anti-MSLN TFP T cells is administered as a single agent.

Embodiment 7: The method of embodiment 1, wherein the dose of anti-MSLN TFP T cells is about 5×107/m2.

Embodiment 8: The method of embodiment 1, wherein the dose of anti-MSLN TFP T cells is about 1×108/m2.

Embodiment 9: The method of embodiment 1, wherein the dose of anti-MSLN TFP T cells is about 5×108/m2.

Embodiment 10: The method of embodiment 1, wherein the dose of anti-MSLN TFP T cells is about 1×109/m2.

Embodiment 11: The method of embodiment 1, wherein a dose range of ±15% of the target dose may be administered.

Embodiment 12: The method of embodiment 3, wherein the second dose of anti-MSLN TFP T cells is administered no sooner than 60 days and no later than 12 months following administration of the first dose of anti-MSLN TFP T cells.

Embodiment 13: The method of embodiment 1, further comprising the step of administering to the patient a lymphodepleting chemotherapy regimen prior to administration of the first dose of anti-MSLN TFP T cells.

Embodiment 14: The method of embodiment 13, wherein the lymphodepleting chemotherapy regimen comprises administration of four doses of fludarabine and three doses of cyclophosphamide.

Embodiment 15: The method of embodiment 13, wherein the lymphodepleting chemotherapy comprises fludarabine administered to the patient at a level of 30 mg/m2/day on days −7 through −4 relative to administration of anti-MSLN TFP T cells, and further comprises cyclophosphamide administered at a level of 600 mg/m2/day on days −6 through −4 relative to administration of anti-MSLN TFP T cells.

Embodiment 16: The method of embodiment 1, further comprising administration of a chemotherapeutic agent.

Embodiment 17: The method of embodiment 16, wherein the chemotherapeutic agent is administered four times at three dose levels, comprising a first dose, a second dose, a third dose, and a fourth dose.

Embodiment 18: The method of embodiment 16, wherein the first dose of the chemotherapeutic agent is administered three weeks after administration of anti-MSLN TFP T cells, and wherein subsequent doses are administered every three weeks thereafter.

Embodiment 19: The method of embodiment 16, wherein the chemotherapeutic agent is administered every three weeks.

Embodiment 20: The method of embodiment 16, wherein the chemotherapeutic agent comprises chemotherapy.

Embodiment 21: The method of embodiment 16, wherein the chemotherapeutic agent comprises pembrolizumab.

Embodiment 22: A method for the treatment of a human patient diagnosed with a mesothelin (MSLN)-expressing unresectable, metastatic, or recurrent cancer, the method comprising the steps of: administering to the patient a lymphodepleting chemotherapy regimen; administering to the patient a plurality of doses, each dose comprising an amount of transduced anti-MSLN TFP T cells T cells, at an interval less than about sixty days between doses; and optionally administering to the patient an effective amount of a chemotherapeutic agent.

Embodiment 23: The method of embodiment 22, wherein the cancer comprises malignant pleural mesothelioma (MPM), non-small cell lung cancer (NSCLC), serous ovarian adenocarcinoma, or cholangiocarcinoma.

Embodiment 24: The method of embodiment 22, comprising administering four doses of anti-MSLN TFP T cells in evenly spaced increments, comprising a first dose, a second dose, a third dose, and a fourth dose.

Embodiment 25: The method of embodiment 24, wherein each dose comprises from about 5×107 to about 1×109 transduced cells/m2.

Embodiment 26: The method of embodiment 25, wherein each dose is about 5×107/m2.

Embodiment 27: The method of embodiment 25, wherein each dose about 1×108/m2.

Embodiment 28: The method of embodiment 25, wherein each dose is about 5×108/m2.

Embodiment 29: The method of embodiment 25, wherein each dose is about 1×109/m2.

Embodiment 30: The method of embodiment 22, wherein each dose comprises a dose range of ±15% of the target dose may be administered.

Embodiment 31: The method of embodiment 24, wherein the second dose of anti-MSLN TFP T cells is administered no sooner than 60 days and no later than 12 months following administration of the first dose of anti-MSLN TFP T cells.

Embodiment 32: The method of embodiment 22, wherein the dose is administered via intravenous infusion.

Embodiment 33: The method of embodiment 22, further comprising the step of administering to the patient a lymphodepleting chemotherapy regimen prior to administration of the first dose of anti-MSLN TFP T cells.

Embodiment 34: The method of embodiment 33, wherein the lymphodepleting chemotherapy regimen comprises administration of four doses of fludarabine and three doses of cyclophosphamide.

Embodiment 35: The method of embodiment 33, wherein the lymphodepleting chemotherapy comprises fludarabine administered to the patient at a level of 30 mg/m2/day on days −7 through −4 relative to administration of anti-MSLN TFP T cells, and further comprises cyclophosphamide administered at a level of 600 mg/m2/day on days −6 through −4 relative to administration of anti-MSLN TFP T cells.

Embodiment 36: The method of embodiment 33, further comprising administering a chemotherapeutic agent, wherein the chemotherapeutic agent is administered four times at three dose levels, comprising a first dose, a second dose, a third dose, and a fourth dose.

Embodiment 37: The method of embodiment 36, wherein the first dose of the chemotherapeutic agent is administered three weeks after administration of anti-MSLN TFP T cells, and wherein subsequent doses are administered every three weeks thereafter.

Embodiment 38: The method of embodiment 36, wherein the chemotherapeutic agent is administered every three weeks.

Embodiment 39: The method of embodiment 36, wherein the chemotherapeutic agent comprises chemotherapy.

Embodiment 40: The method of embodiment 36, wherein the chemotherapeutic agent comprises pembrolizumab.

### EXAMPLES

The present disclosure is further described in detail by reference to the following experimental examples. These examples are provided for purposes of illustration only, and are not intended to be limiting unless otherwise specified. Thus, the present disclosure should in no way be construed as being limited to the following examples, but rather, should be construed to encompass any and all variations which become evident as a result of the teaching provided herein. Without further description, it is believed that one of ordinary skill in the art can, using the preceding description and the following illustrative examples, make and utilize the compounds of the present disclosure and practice the claimed methods. The following working examples specifically point out various aspects of the present disclosure, and are not to be construed as limiting in any way the remainder of the disclosure.

### Example 1

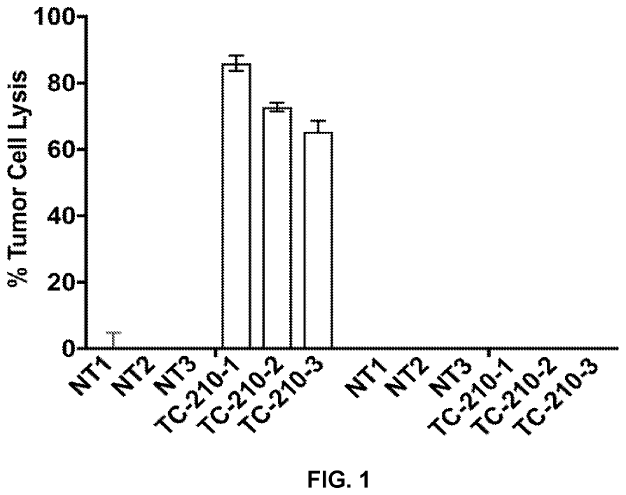

10⁵ anti-MSLN TFP or non-transduced (NT) cells were co-cultured with labeled with 10⁴ MSTO-MSLN-LUC (mesothelin positive) or U251-LUC (mesothelin negative) tumor cells and incubated for 20 hours at 37° C., 5% CO₂. Luciferase activity was measured on cell lysates using SpectraMax Multi-mode Microplate ReaderCells. Bars in FIG. 1 shows the average percentage of cell lysis for 3 wells±standard deviation. Results indicate that anti-MSLN TFP T cells produce IFNγ and IL2 in response to mesothelin expressing tumor cells.

### Example 2: Anti-Tumor and Pro-Inflammatory Effects of Anti-MSLN TFP are MSLN Dependent

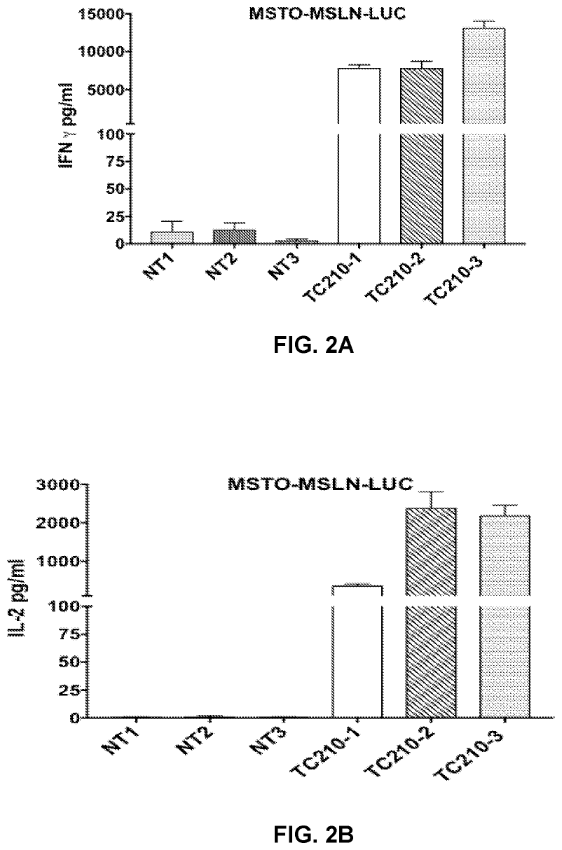

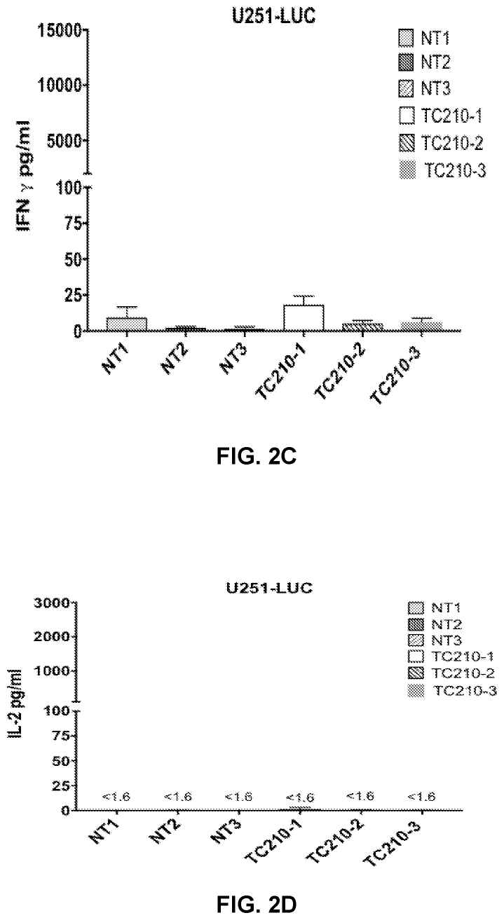

10⁵ anti-MSLN TFP T cells or NT cells were co-cultured with 10⁴ MSTO-MSLN-LUC (mesothelin positive) or U251-LUC (mesothelin negative) tumor cells at 37° C., 5% CO₂. Culture supernatants were collected after ˜20 hours and IFNg and IL-2 were measured by Luminex-based assays. Bars in FIG. 2 show average percentage of cytokine concentration in pg/mL for triplicate wells±standard deviation. These results indicate that anti-tumor and pro-inflammatory effects of anti-MSLN TFP were mesothelin expression dependent.

### Example 3: Primary Mesothelioma Tumor Nod SCID Gamma (NSG) Animal Model

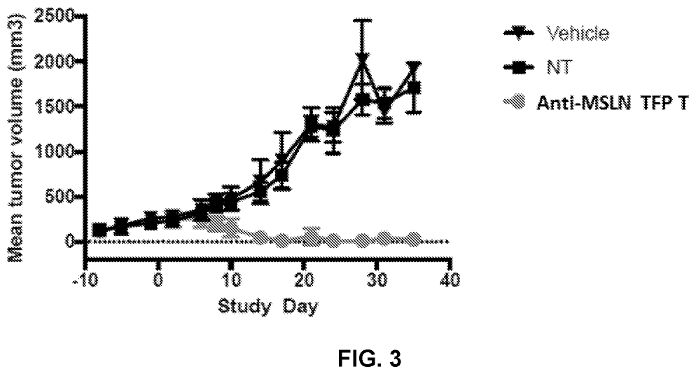

Primary mesothelioma NSG animal models were developed using a cell line originally isolated from a biphasic malignant pleural mesothelioma, which were subsequently engineered to overexpress mesothelin (MSTO-MSLN). NSG mice (n=21) were injected s.c. in the hind flank with 10⁶ MSTO-211H-FL MSLN tumor cells. Growth of mesothelioma MSTO-211HFL MSLN in NSG mice treated with T cells as shown by s.c. caliper tumor measurement of mice. Tumors were measured using calipers and tumor volume determined. When tumors reached a size of 200 mm³, mice were infused with either anti-MSLN TFP T cells or non-transduced (NT) T cells and Tumor growth was monitored in the tumor bearing animals. As shown in FIG. 3, treatment of tumor bearing animals with anti-MSLN TFP T cells showed rapid tumor control and eventual clearance by day 25 post treatment. In contrast, infusion of NT T cells resulted in unabated tumor growth, until animals became moribund. Tumor-bearing mice treated with either NT T cells or vehicle had progressive tumor growth (0/5 and 0/9 tumor-free mice for vehicle and NT, respectively), while mice treated with anti-MSLN TFP had regression of tumor in 7/9 mice.

### Example 4: Persistence and Efficacy of Anti-MSLN TFP T Cells after MSTO-MSLN Tumor Clearance in Mice

Animals that had previously been treated with anti-MSLN TFP T cells and cleared primary mesothelioma tumors (MSTO-MSLN), were split into 2 groups, and subsequently re-challenged with either MSTO-MSLN or the parental low expressing MSTO cells in the contralateral flank to the site of the primary tumor challenge. anti-MSLN TFP T cells were injected into the animals with established mesothelioma. On study day 56, 4 weeks after complete tumor clearance, anti-MSLN TFP injected mice were rechallenged with MSTO (low msln) (panel A) or MSTO-MSLN (high MSLN) (panel B) at 10⁶ cells per mouse. Naïve mice were implanted with the same dose of each of the tumor cell lines. Tumor volume was monitored by caliper measurements twice a week.

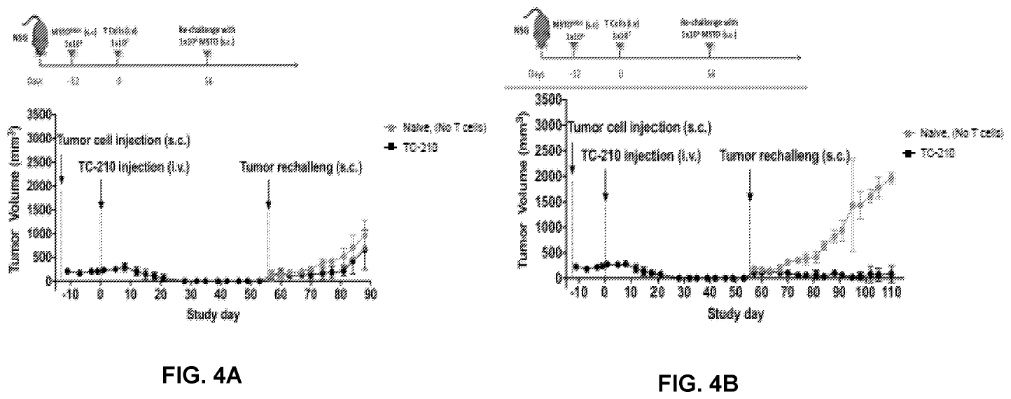

Animals that had been re-challenged with MSTO-MSLN cells showed rapid and robust tumor control, whereas the control MSTO-mesothelin low tumors were observed to grow (FIG. 4). Together, the data show that anti-MSLN TFP T cells are not only very efficacious at eliminating primary mesothelin expressing tumors, but also that the anti-MSLN TFP shows long-lasting persistence and function. The effect exists long after primary tumor clearance and is associated with maintenance of functionality, with re-challenge experiments showing not only clear tumor eradication, reflecting proliferation and lytic functions, but that anti-MSLN TFP T cells are also capable of promoting immune surveillance, trafficking to sites distal from the primary tumor, and mediating anti-tumor activity.

### Example 5

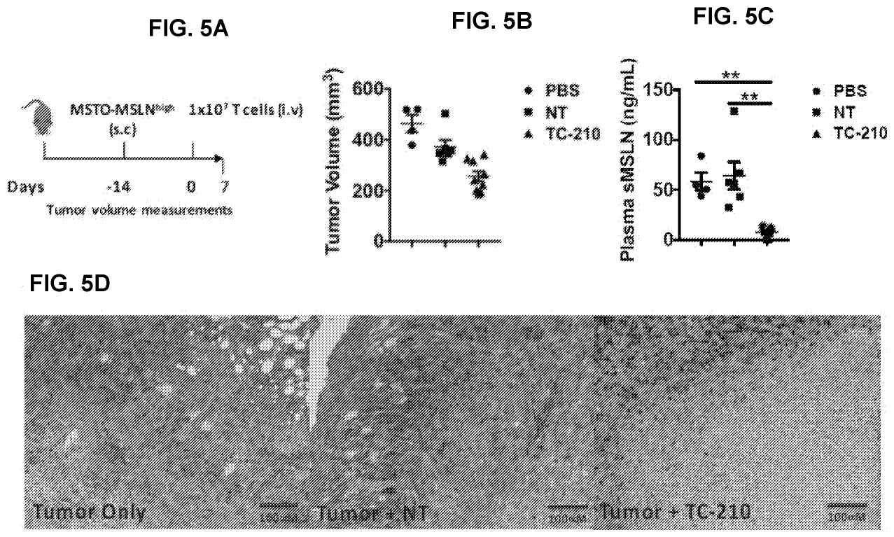

Peripheral blood and tumors were collected from MSTO-MSLN tumor bearing animals seven days post anti-MSLN TFP or NT T cell infusion. Tumor volumes were measured on day 6 after injection with T cells. The level of soluble MSLN (sMSLN) in plasma was measured on day 7 after T cell injection. Circulating levels of soluble mesothelin were reduced in anti-MSLN TFP Treated animals. Tumors harvested on day 7 after injection were examined by immunohistochemistry, which clearly showed T cell infiltration in tumors from anti-MSLN TFP T cell treated animals. This infiltration correlated with a reduction in mesothelin staining in these tumors (FIG. 5). Flow cytometry evaluation of the peripheral blood showed a strong population of anti-MSLN TFP T cells in the circulation and serum cytokine analysis confirmed T cell activity, with elevations in effector cytokines (e.g., IFNγ, TNF, granulocyte-macrophage colony-stimulating factor [GM-CSF], IL-1) and lytic molecules (granzyme).

### Example 6: Non-Small Cell Lunt Carcinoma Model

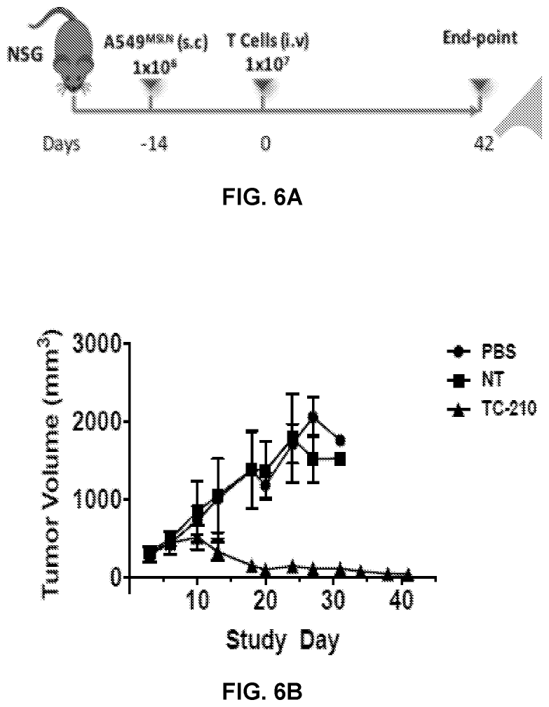

The efficacy of anti-MSLN TFP T cells was addressed in an NSCLC model. NSG mice were subcutaneously seeded with the A549 lung carcinoma cell line engineered to express high levels of MSLN (FIG. 6). Tumor bearing mice were treated with PBS, NT T cells or anti-MSLN TFP T cells. Anti-MSLN TFP T cell treatment resulted in tumor control and recession between days 7 to 10 post infusion and complete tumor regression were achieved by day 20 post infusion and were maintained until the endpoint of the study (day 42 post fusion). No tumor regression was observed in PBS or NT T cell treated groups (FIG. 6). Anti-MSLN TFP T cell anti-tumor activity correlated with anti-MSLN TFP T cell expansion, elevated levels of inflammatory cytokines (IFN-g, IL-2, IL-5, IL-6, IL-10, sCD137, GM-CSF, and tumor necrosis factor alpha), as well as cytolytic payload proteins (Granzyme A and Granzyme B). The production kinetics of all cytokines, except IL-2, correlated with the tumor control observed for anti-MSLN TFP T cells. These data indicate that anti-MSLN TFP T cells expand and generate a potent anti-tumor response in an NSG NSCLC model.

### Example 7: Ovarian (OVCAR3) Adenocarcinoma NSG Model

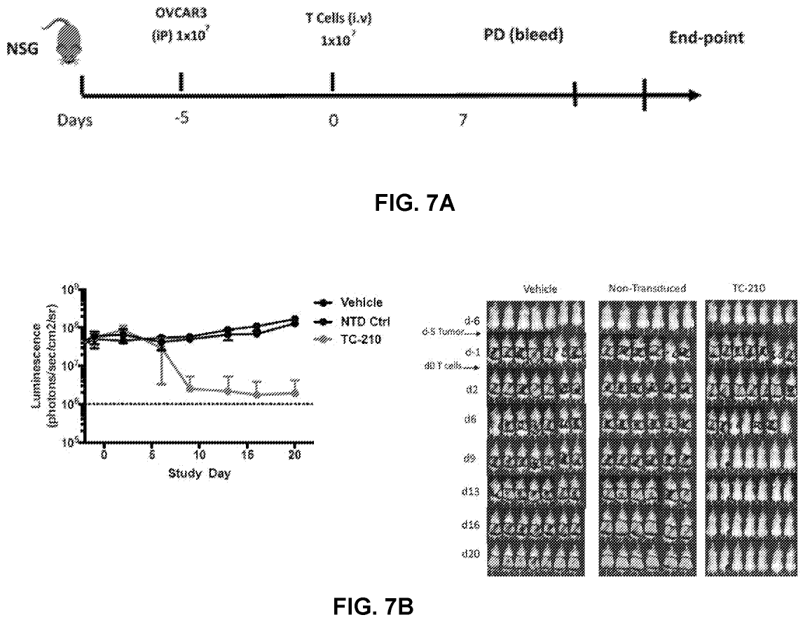

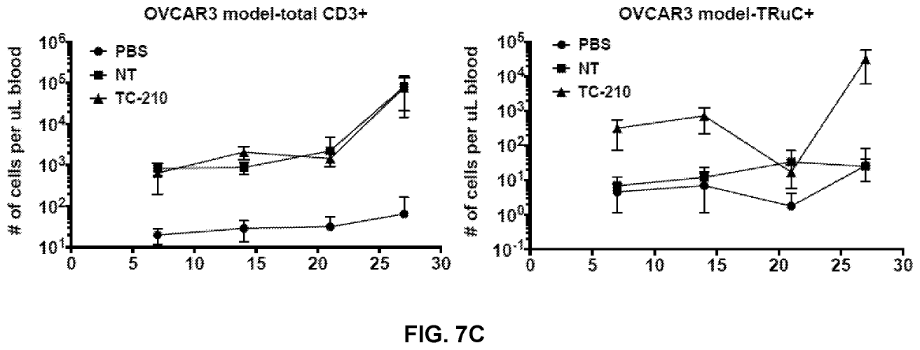

An ovarian adenocarcinoma NSG model was developed using the OVCAR3 cell line. This cell line expresses luciferase, which enables tracking and monitoring disease longitudinally with bioluminescence imaging (BLI) (FIG. 7). Intraperitoneal (IP) delivery of OVCAR3 cells was conducted to better reflect the clinical condition. NSG mice were implanted IP with 10⁷ OVCAR3-luc tumor cells. Five days later, mice were injected intraperitoneally (IP) with 4×10⁶ anti-MSLN TFP-transduced T cells (1×10⁷ total T cells), NT T cells (1×10⁷ total T cells) or vehicle. Mice were injected IP with luciferin (150 mg/kg) and imaged using IVIS to monitor tumor growth. Tumor-bearing mice treated with either NT T cells or vehicle had progressive tumor growth, while mice treated with anti-MSLN TFP had regression of tumor in 6/7 mice. When tumor burden reached luminesce of 5×10⁷, animals were treated with either NT or anti-MSLN TFP T cells. As shown in FIG. 7, anti-MSLN TFP Treatment significantly reduced the disease burden in most mice when compared to vehicle or NT T cells.

### Example 8: Phase 1/II Clinical Trial of Anti-MSLN TFP

This example provides details of a first-in-human phase 1/II open label clinical study to evaluate the safety and efficacy of autologous genetically engineered anti-MSLN TFP T cells in subjects with advanced mesothelin-positive cancers.

### Phase 1 Primary Objectives

Evaluate the safety of autologous genetically modified anti-MSLN TFP T cells in subjects with mesothelin-expressing unresectable, metastatic, or recurrent cancers; and establish the recommended phase 2 dose (RP2D) according to dose-limiting toxicity (DLT) of defined adverse events (AEs).

### Phase 1 Secondary Objectives

Determine the ORR: complete response (CR)+PR according to Response Evaluation Criteria in Solid Tumors RECIST) v 1.1 and response duration when anti-MSLN TFP T cells are administered with or without a lymphodepletion regimen; evaluate the efficacy of autologous genetically modified anti-MSLN TFP T cells in subjects with mesothelin-expressing unresectable, metastatic, or recurrent cancers as assessed by time to response (TTR), duration of response (DoR), PFS, and OS; and develop and validate an in vitro diagnostic (IVD) assay for the screening of mesothelin expression for regulatory approval.

### Phase 2 Primary Objectives

To evaluate the efficacy of autologous genetically modified anti-MSLN TFP T cells in subjects with mesothelin-expressing unresectable, metastatic, or recurrent cancers; and endpoints: overall response rate (ORR: CR+PR) according to RECIST v1.1.

### Phase 2 Secondary Objectives

To evaluate the efficacy of autologous genetically modified anti-MSLN TFP T cells in subjects with mesothelin-expressing unresectable, metastatic, or recurrent cancers as assessed by TTR, DoR, PFS, and OS; to assess whether subjects who experience progressive disease following anti-MSLN TFP cell therapy experience a response upon a second infusion; and to develop and validate an in vitro diagnostic (IVD) assay for the screening of mesothelin expression for regulatory approval.

### Phase 2 Exploratory Objectives

To evaluate the correlation between resistance or response to anti-MSLN TFP T cell therapy with anti-MSLN TFP T cell expansion, persistence, phenotype, and functionality; to evaluate candidate biomarkers in tumor tissue, including anti-MSLN TFP T cell infiltration and pre and post evaluation of the tumor microenvironment and measurement of immune cell markers, and correlation with clinical response to treatment; and to evaluate changes in health-related quality of life following treatment with anti-MSLN TFP T cells.

### Overall Study Design

The purpose of the clinical study is to evaluate the safety and efficacy of autologous genetically engineered anti-MSLN TFP T cells in subjects with advanced mesothelin-positive cancers. The study is completed when the last subject that responds to anti-MSLN TFP T cell treatment has been followed for 24 months, or when the last patient infused with anti-MSLN TFP T cells withdraws consent, experiences disease progression, dies, or is lost to follow-up, whichever occurs last. All subjects are transferred to a dedicated long-term follow-up (LTFU) protocol to be monitored for gene therapy-related delayed adverse events for 15 years (from initial date of anti-MSLN TFP T cell infusion), in accordance with FDA regulatory requirements for gene therapy clinical trials.

Subjects are screened for general health, performance status, diagnosis, disease stage, and mesothelin expression.

Subjects are aged ≥18 years with a diagnosis of MPM, NSCLC, ovarian cancer, or cholangiocarcinoma confirmed by central histology assessment. Subjects undergo testing to determine adequatepresence of mesothelin expression on the tumor.

Following screening, subjects meeting all eligibility will undergo a large-volume leukapheresis at the enrolling institution to obtain cells for the manufacture of autologous anti-MSLN TFP T cells. Subject peripheral blood mononuclear cells (PBMC) is collected and processed at the site for T cell selection, gene transduction, activation, and expansion.

Frozen leukocytes are shipped centrally for further processing. Then, anti-MSLN TFP T cells (lentivirally transduced T cells) is formulated, cryopreserved, and shipped back to the enrolling institution for infusion.

The phase 1 portion of the study evaluates 4 doses of anti-MSLN TFP T cells preceded (Dose Levels 1, 3, 5, and 7) or not (Dose Levels 0, 2, 4, and 6) by a lymphodepleting chemotherapy regimen with fludarabine for 4 days (day −7 to day −4) and cyclophosphamide for 3 days (day −6 through day −4). 16 to 28 subjects are treated during the dose escalation phase.

For dose levels without lymphodepletion (Dose Levels 0, 2, 4, and 6), dose escalation proceeds in single patient cohorts to identify the RP2D. Should the first subject enrolled in a dose level without lymphodepletion develop a grade ≥3 toxicity presumably related to anti-MSLN TFP T cell infusion, the single patient cohort is expanded to a 3-patient cohort and proceed according to a 3+3 dose escalation design. If the initial dose level (i.e., DLO) is deemed not safe, a lower dose of 1×107 m² is evaluated (DL-1). For dose levels with lymphodepletion (Dose Levels 1, 3, 5, and 7), a standard 3+3 dose escalation strategy is used to identify the recommended phase 2 dose (RP2D). Prior to the administration of protocol-defined therapy all eligibility criteria are reconfirmed, and a baseline tumor assessment obtained. Subjects receive Granulocyte Colony Stimulating Factor (G-CSF) support starting 24 hours after lymphodepleting chemotherapy until resolution of neutropenia.

Both the lymphodepleting chemotherapy as well as the anti-MSLN TFP T cell infusion may either be given as an outpatient treatment or subjects may be hospitalized at the discretion of the Investigator. For the phase 1 portion of the study, all subjects are admitted for observation overnight following the inpatient administration of anti-MSLN TFP T cells. The administration of anti-MSLN TFP T cells (if deemed safe in the phase 1 portion of the study) may take place on an outpatient setting at the discretion of the Investigator.

### Replacement of Subjects

Subjects who withdraw from the study prior to initiation of lymphodepleting chemotherapy may be replaced.

If the transduced T cell dose is less than the protocol specified dose, manufacturing of additional transduced T cells from excess banked leukapheresis product are undertaken to achieve a total dose in the target range. In the event that no banked leukapheresis product is available a second leukapheresis may be performed. If, after a second leukapheresis and manufacturing attempt, an anti-MSLN TFP T cell dose fails to meet the minimum dose requirement, subjects may still be eligible to receive anti-MSLN TFP T cells and participate on this trial. However, an additional subject whose anti-MSLN TFP T cell dose meets the minimum cell dose requirement is added to the cohort.

Subjects treated in the phase 2 portion of the study who have a confirmed response or have SD for >4 months and then progress may receive a second anti-MSLN TFP infusion, provided eligibility criteria are met again, including adequate mesothelin expression. The second infusion is administered following the same guidelines that rule the administration of the first one. Subjects meeting eligibility criteria may receive a second anti-MSLN TFP T cell infusion no sooner than 60 days and no later than 12 months following completion of the first anti-MSLN TFP T cell infusion.

### Study Duration and Completion

For each individual subject, the length of study participation includes an up to 28-day screening period that would account for the time from signing the pre-screening informed consent form to determining full eligibility and enrollment, a 4-day conditioning chemotherapy treatment period (as applicable), an anti-MSLN TFP T cell treatment period (which may include an in-hospital period), and a post treatment assessment period lasting a maximum of 24 months. Thus, for subjects who complete the entire protocol from the date of informed consent through the completion of the 24-months of follow up post anti-MSLN TFP T cell infusion, the duration of the study is approximately 2 years and 2 months. However, individual study duration varies depending on a subject's screening requirements, response to treatment, and survival. The study is completed when the last subject that responds to anti-MSLN TFP T cell treatment has been followed for 24 months, or when the last patient infused with anti-MSLN TFP T cells withdraws consent, experiences disease progression, dies, or is lost to follow-up, whichever occurs last.

All subjects are transferred to a dedicated long-term follow-up (LTFU) protocol in order to be monitored for gene therapy-related delayed adverse events for 15 years (from initial date of anti-MSLN TFP T cell infusion), in accordance with FDA regulatory requirements for gene therapy clinical trials. Transfer to the LTFU protocol occurs when patients complete 24 months of follow-up post anti-MSLN TFP T cell infusion, or when they withdraw consent from the current protocol or experience disease progression, whichever comes first. All subjects continue to be followed for overall survival on the LTFU protocol.

The primary analyses is conducted when all subjects in the phase 1 portion of the study have been followed for safety for at least 28 days post anti-MSLN TFP T cell infusion and when all subjects enrolled in the phase 2 portion of the study complete the 6-month disease response assessment, are lost to follow-up, withdraw from the study, or die, whichever occurs first.

### Subject Eligibility

Subjects are assessed for and must meet eligibility criteria for study participation (ie, at screening) AND again prior to first protocol defined therapy (i.e., at baseline) unless otherwise specified below.

### Inclusion Criteria

A subject must meet the following inclusion criteria to be eligible for participation in the study:

Subject (or legally authorized representative) has voluntarily agreed to participate by giving written informed consent in accordance with ICH Good Clinical Practice (GCP) guidelines and applicable local regulations.

Subject has agreed to abide by all protocol required procedures including study related assessments, and management by the treating institution for the duration of the study and LTFU.

Subject is ≥18 years of age at the time the Informed Consent is signed.

Subject has a pathologically confirmed diagnosis (for screening: fresh tissue preferred but banked tumor biopsy allowed if obtained within the prior 12 months. Note: cytology is insufficient) of either MPM, serous ovarian adenocarcinoma, cholangiocarcinoma, or NSCLC.

Subject's tumor has been pathologically reviewed with confirmed positive mesothelin expression on ≥50% of tumor cells that are 2+ and/or 3+ by immunohistochemistry.

Subject has advanced (ie, metastatic or unresectable) cancer. Unresectable refers to a tumor lesion in which clear surgical excision margins cannot be obtained without leading to significant functional compromise.

Subject has at least 1 evaluable and measurable lesion as defined by RECIST v 1.1 after the mandatory pre-anti-MSLN TFP T cell infusion fresh tissue biopsy has been performed. Subjects who have received prior local therapy (including but not limited to embolization, chemoembolization, radiofrequency ablation, or radiation therapy) are eligible provided measurable disease falls outside of the treatment field or within the field and has shown >20% growth in size since post-treatment assessment.

Prior to anti-MSLN TFP T cell infusion, subjects must have received at least 1 systemic standard of care therapy for metastatic or unresectable disease (unless otherwise specified), as follows:

### MPM

Subjects must have received standard first line therapy with a platinum-based regimen or they must have elected not to pursue frontline standard of care therapy. The subject must not have required a paracentesis within the preceding 4 weeks nor be projected to require a paracentesis within the next 8 weeks.

### NSCLC

Subjects must have a pathologically confirmed (by histology or cytology) diagnosis of NSCLC, which is currently Stage 3B or Stage 4 disease. A subject with non-squamous NSCLC must have been tested for relevant EGFR mutations, ALK translocation or other genomic aberrations (e.g., ROS rearrangement, BRAF V600E mutation) for which FDA-approved targeted therapy is available and, if positive, the subject should have received at least one such therapy prior to study enrollment. For subjects without an actionable mutation, the subject must have received a currently approved frontline regimen (e.g., immune checkpoint inhibitor-based therapy).

### Serous Ovarian Adenocarcinoma

The subject must have a histologically confirmed diagnosis of recurrent serous ovarian adenocarcinoma, which is currently Stage 3 or Stage 4 disease. A histologic diagnosis of borderline, low malignant potential epithelial carcinoma is not permitted. The subject must not have required a paracentesis within the preceding 4 weeks nor be projected to require a paracentesis within the next 8 weeks. The subject has no evidence of a bowel obstruction within the last 8 weeks.

### Cholangiocarcinoma

Subjects must have received at least one standard systemic regimen for unresectable or metastatic disease (e.g., gemcitabine- or 5-FU-containing regimens) or they must have elected not to pursue frontline standard of care therapy.

Subjects must have measurable disease, defined as at least one lesion that can be accurately measured in at least one dimension (longest diameter to be recorded for non-nodal lesions and short axis for nodal lesions) as ≥20 mm (≥2 cm) with conventional techniques or as ≥10 mm (≥1 cm) with computed tomography (CT) scan or magnetic resonance imaging (MRI). Subjects who have received systemic adjuvant chemotherapy are permitted. Subject has an Eastern Cooperative Oncology Group (ECOG) Performance Status 0 or 1. Subject has a left ventricular ejection fraction >45% as measured by resting echocardiogram, with no clinically significant pericardial effusion. Subject is fit for leukapheresis and has adequate venous access for the cell collection.

Female patients of childbearing potential (FCBP) must have a negative urine or serum pregnancy test (FPCP is defined as premenopausal and not surgically sterilized). FCBP must agree to use effective birth control or to abstain from heterosexual activity throughout the study, starting on the day of first dose of lymphodepleting chemotherapy through 12 months post anti-MSLN TFP T cell infusion or for 4 months after there is no evidence of persistence of gene modified cells in the blood, whichever is longer. Effective contraceptive methods include intra-uterine device, oral or injectable hormonal contraception, or 2 adequate barrier methods (e.g., diaphragm with spermicide, cervical cap with spermicide, or female condom with spermicide). Spermicides alone are not an adequate method of contraception.

Male subjects are surgically sterile or agree to use a double barrier contraception method or abstain from heterosexual activity with a female of childbearing potential starting at the first dose of protocol-defined treatment and for 4 months thereafter or longer (if indicated in the country specific monograph/label for cyclophosphamide).

Subject must have adequate organ function as indicated by the laboratory values in Table 2.

TABLE 2
Laboratory Values Indicating Sufficient Organ Function

| System | Laboratory Value |
|---|---|
| Hematological | |
| Absolute neutrophil count (ANC) | ≥1 × 10⁹/L (without G-CSF support) |
| Absolute lymphocyte count (ALC) | ≥0.3 × 10⁹/L |
| Platelets | ≥100 × 10⁹/L |
| Hemoglobin | ≥90 g/L (without transfusion support within |
| | 7 days prior to leukapheresis) |
| Coagulation | |
| Prothrombin time or international | ≤1.5 × upper limit of normal (ULN) |
| normalized ratio | |
| Partial thromboplastin time (PTT) | ≤1.5 × upper limit of normal (ULN) |
| Renal | |
| Calculated or measured creatinine clearance^{a} | ≥40 mL/min |
| Hepatic | |
| Serum total bilirubin | ≤2 × ULN (unless subject has documented |
| | Gilbert's syndrome with direct bilirubin < 35% |
| | of total bilirubin or unless secondary to bile duct |
| | obstruction by tumor) |
| Alanine aminotransferase (ALT) | ≤2.5 × ULN or ≤5 × ULN if documented liver |
| | metastasis |
| Serum glutamic pyruvic transaminase (SGPT) | ≤2.5 ≤ULN or ≤5 × ULN if documented liver |
| | metastasis |

^{a}= Screening (Visit 1) creatinine clearance is estimated using the Cockcroft-Gault formula: (140 − age) × weight kg Creatinine clearance = 72 × serum creatinine mg/dl (×0.85 in females)
Renal function is re-assessed at Baseline (visit 3). Subjects ≥ 65 years of age must have renal function measured either by 24-hour urine creatinine collection OR by nuclear medicine glomerular filtration rate measurement, according to standard practice at the treating institution. Subjects < 65 years of age may have creatinine clearance estimated using the Cockcroft-Gault formula.

### Exclusion Criteria

A subject meeting any of the following exclusion criteria is not be eligible for participation in the study:
Inability to follow the procedures of the study (e.g., due to language problems, psychological disorders, dementia, confusional state, etc). Known or suspected noncompliance, drug or alcohol abuse.
Participation in another study with investigational drug within the 28 days or 5 half-lives of the drug, whichever is shorter, preceding and during the present study.
Subject is pregnant (or intends to become pregnant during the course of the study) or breastfeeding.
Subject has received the following therapy/treatment within the specified timeframe prior to initiating protocol-defined treatment with either lymphodepletion or anti-MSLN TFP T cells:
   Cytotoxic chemotherapy within 3 weeks of leukapheresis or (with the exception of lymphodepleting chemotherapy) within 3 weeks of anti-MSLN TFP T cell infusion.
   Corticosteroids: therapeutic doses of steroids are stopped >72 hours prior to leukapheresis and at least 2 weeks prior to anti-MSLN TFP T cell infusion. Use of inhaled steroids or topical cutaneous steroids is not exclusionary. Corticosteroid therapy at a pharmacologic dose (≥5 mg/day of prednisone or equivalent doses of other corticosteroids) and other immunosuppressive drugs is avoided until 3 months after anti-MSLN TFP administration, unless medically indicated to treat new toxicity. Physiological replacement doses of steroids (up to 5 mg/day of prednisone equivalent) may be allowed.
   Immunosuppression: any other immunosuppressive medication is stopped ≥4 weeks prior to enrollment, including calcineurin inhibitors, methotrexate or other chemotherapy drugs, mycophenolyate, steroids (e.g., as described above), rapamycin, thalidomide, or immunosuppressive antibodies such as rituximab, anti-TNF, anti-IL6 or anti-IL6R.
   Use of an anti-cancer vaccine within 2 months in the absence of tumor response. The subject is excluded if their disease is responding to an experimental vaccine given within 6 months;
   Any previous gene therapy using an integrating vector; TKI (e.g., EGFR inhibitors) within 72 hours;
   Any previous allogeneic hematopoietic stem cell transplant;
   Investigational treatment or clinical trial within 4 weeks or 5 half-lives of investigational product, whichever is shorter;
   Radiotherapy to the target lesions within 3 months prior to lymphodepleting chemotherapy. A lesion with unequivocal progression may be considered a target lesion regardless of time from last radiotherapy dose. NOTE: There is no washout period for palliative radiation to non-target lesions;
   Hepatic radiation, chemoembolization, and/or radiofrequency ablation within 4 weeks.
   Current anticoagulative therapy.
Toxicity from previous anti-cancer therapy that has not recovered to ≤grade 1 (except for nonclinically significant toxicities, e.g., alopecia, vitiligo). Subjects with grade 2 toxicities that are deemed stable or irreversible (e.g., peripheral neuropathy) can be enrolled.
History of allergic reactions attributed to compounds of similar chemical or biologic composition to fludarabine, cyclophosphamide, or other agents used in the study.
History of autoimmune or immune mediated disease such as multiple sclerosis, lupus, rheumatoid arthritis, inflammatory bowel disease, or small vessel vasculitis.
Major surgery (other than diagnostic surgery) within 4 weeks prior to enrollment, minor surgery including diagnostic surgery within 2 weeks (14 days) excluding central intravenous port placements and needle aspirate/core biopsies. Radio frequency ablation or transcatheter arterial chemoembolization within 6 weeks prior to enrollment.
Biliary stents
Central nervous system (CNS) disease/brain metastases:
   Subjects with leptomeningeal disease, carcinomatous meningitis, or symptomatic CNS metastases: subjects are eligible if they have completed their treatment, have recovered from the acute effects of radiation therapy or surgery prior to study entry, and a) have no evidence of brain metastases post treatment or b) are asymptomatic, have discontinued corticosteroid treatment or anti-seizure medications for these metastases for at least 4 weeks and have radiographically stable CNS metastases without associated edema or shift for at least 3 months prior to study entry (note: prophylactic anti-seizure medications are acceptable; up to 5 mg/day of prednisone or equivalent is allowed).
Subject has any other prior or concurrent malignancy with the following exceptions:
   Adequately treated basal cell or squamous cell carcinoma (adequate wound healing is required prior to study entry)
   In situ carcinoma of the cervix or breast, treated curatively and without evidence of recurrence for at least 3 years prior to the study
   A primary malignancy which has been completely resected and in complete remission for ≥5 years
Subject has an electrocardiogram (ECG) showing a clinically significant abnormality at screening or showing an average QTc interval >450 msec in males and >470 msec in females (>480 msec for subjects with bundle branch block). Either Fridericia's or Bazett's formula may be used to correct the QT interval.
Subject has uncontrolled intercurrent illness including, but not limited to:
   Ongoing or active infection: e.g., sepsis, prolonged unresolved bacterial cholangitis with destruction of bile duct branches (e.g., after endoprosthesis insertion) or 2 or more cholangitis in the last 6 months;
   Clinically significant cardiac disease defined by congestive heart failure New York Heart Association class 3 or class 4;
   Uncontrolled clinically significant arrhythmia;
   Acute coronary syndrome (angina or myocardial infarction), stroke, or peripheralvascular disease in the last 6 months;
   Interstitial lung disease (subjects with existing pneumonitis as a result of radiation are not excluded; however, subjects must not be oxygen dependent as demonstrated by oxygen saturation <92% on room air);
   Liver cirrhosis or primary sclerosing cholangitis
Subject has active infection with human immunodeficiency virus (HIV), hepatitis B virus, hepatitis C virus (HCV), or human T-lymphotropic virus (HTLV) as defined below:
   Positive serology for HIV, HTLV-1, or HTLV-2;
   Active hepatitis B infection as demonstrated by test for hepatitis B surface antigen. Subjects who are hepatitis B surface antigen negative but are hepatitis B core antibody positive must have undetectable hepatitis B deoxyribonucleic acid (DNA) and receive prophylaxis against viral reactivation;
   Active hepatitis C infection as demonstrated by hepatitis C ribonucleic acid (RNA) test. Subjects who are HCV antibody positive are screened for HCV RNA by any reverse transcription polymerase chain reaction (PCR) or bDNA assay. If HCV antibody is positive, eligibility is determined based on a negative screening RNA value.

### Concomitant Treatments

### Study Treatment and Concomitant Therapy

During the course of the study, investigators may prescribe any concomitant medications or treatment deemed necessary to provide adequate supportive care except those medications listed in the excluded medication below. All concurrent therapies, including medications and supportive therapy (e.g., intubation, dialysis, and blood products), are recorded from the date the subject is enrolled into the study through 3 months after completing anti-MSLN TFP Therapy. After 3 months post anti-MSLN TFP T cell infusion, only targeted concomitant medication is collected, including immunosuppressive drugs, anti-infective drugs, vaccinations, and any therapy for the treatment of the subject's malignancy for 1 year beyond disease progression. Specific concomitant medication collection requirements and instructions are included in the case report form (CRF) completion guidelines.

### Prohibited Concomitant Medications

In general, medications that might interfere with the evaluation of the investigational product are not used unless absolutely necessary. Medications in this category include (but are not limited to): immunosuppressants and corticosteroid anti-inflammatory agents including prednisone, dexamethasone, solumedrol, and cyclosporine. Treatment for the subject's cancer such as chemotherapy, immunotherapy, targeted agents, radiation, and high dose corticosteroid, other than defined/allowed in this protocol, and other investigational agents are prohibited except as needed for treatment of disease progression. Other prohibited medicines are listed under Exclusion Criteria.

### Study Restrictions

### Contraception

There are no data regarding the safety of anti-MSLN TFP T cells during pregnancy or lactation in humans. Female subjects who are pregnant, intending to become pregnant, or breast feeding are excluded from this study.

Female and male subjects of reproductive potential agree to avoid becoming pregnant or impregnating a partner, respectively. The required duration of contraception is described below:
a. FCBP must agree to use an effective method of contraception starting at the first dose of chemotherapy for at least 12 months thereafter and 4 months after the anti-MSLN TFP gene modified cells are no longer detected in the blood.
b. Male subjects must agree to use an effective method of contraception starting at the first dose of chemotherapy and for 4 months thereafter or longer (if indicated in the country specific monograph/label for cyclophosphamide).

FCBP is defined as premenopausal and not surgically sterilized.

Effective methods of contraception include intra-uterine device, injectable hormonal contraception, oral contraception, or 2 adequate barrier methods (e.g., diaphragm with spermicide, cervical cap with spermicide, or female condom with spermicide—spermicides alone are not an adequate method of contraception).

Abstinence (relative to heterosexual activity) can be used as the sole method of contraception if it is consistently employed as the subject's preferred and usual lifestyle and if considered acceptable by local regulatory agencies and IRBs. Periodic abstinence (e.g., calendar, ovulation, sympto-thermal, post-ovulation methods, etc) and withdrawal are not acceptable methods of contraception.

### Long-Term Follow-Up

All subjects are followed for 15 years from time of anti-MSLN TFP T cell infusion for observation of delayed adverse events in accordance FDA and European Medicines Agency requirements for gene therapy clinical trials (FDA Guidance for Industry: Gene therapy clinical trials—observing subjects for delayed adverse events [November 2006]; CHMP Guideline on follow-up of patients administered with gene therapy medicinal products [October 2009]).

Subjects are seen in the clinic and safety blood samples taken according to the schedule of events at multiple timepoints within the first month following anti-MSLN TFP T cell infusion; at 3, 6, and 12 months during the first year post-infusion; every 6 months in years 2 to 5; and annually from years 6 to 15. Assessments, including medical history, physical examination, delayed adverse events associated with gene therapy, exposure to mutagenic agents, anti-tumor agents, and other medicinal products, are collected initially. However, all subjects are transferred to the LTFU study upon completion of 24 months of follow-up post anti-MSLN TFP infusion, or when subjects withdraw consent from the protocol or experience disease progression, whichever comes first. All subjects continue to be followed for overall survival on the LTFU protocol.

### Example 9: Leukapheresis and Anti-MSLN TFP T Cell Manufacturing

Anti-MSLN TFP T cell product is an engineered autologous ACT. The first step in the manufacturing of anti-MSLN TFP T cell product is the collection of a subject's T cells via leukapheresis. Subjects who complete screening procedures and who meet eligibility criteria are eligible to undergo leukapheresis to obtain starting material for the manufacture of autologous anti-MSLN TFP T cells. A large-volume non-mobilized PBMC collection is performed (12- to 15-liter apheresis) according to Institutional standard procedures for collection of the starting material. The goal is to collect approximately 5 to 10×10⁹ PBMCs (minimum collection goal 1.5×10⁷ PMBC/kg). The leukapheresed cells are then packaged for expedited shipment to the manufacturing facility as described in the investigational product manual. In cases where the minimum number of PBMCs are not collected or the manufacturing of sufficient anti-MSLN TFP T cells is not successful, a second leukapheresis may be performed. Citrate anticoagulant is used during the procedure and prophylaxis against the adverse effects of this anticoagulant (e.g., CaCl₂ infusions) may be employed at the Investigator's discretion. The collected leukapheresis product will then be frozen and transported either the same day or overnight to the approved Cell Processing Facility (CPF) as described in the investigational product manual.

Upon arrival at the CPF, each subject's leukapheresed product is processed to enrich for the T cells containing PBMC fraction. T cells are then stimulated to expand and transduced with a lentiviral vector to introduce the anti-MSLN TFP Transgene to obtain anti-MSLN TFP T cells. Transduced T cells (i.e., anti-MSLN TFP T cells or anti-MSLN TFP T cell product) are then expanded and cryopreserved to generate the investigational product per CPF standard operating procedures (SOPs). Once the anti-MSLN TFP T cell product has passed certain release tests, the CPF ships it back to the treating facility.

The anti-MSLN TFP T cell product is administered first during the phase 1 portion of the study (i.e., the dose escalation phase) as a single infusion at the initial dose 5×10⁷ cells/m² (i.e., dose level zero or DLO). The dose escalation phase evaluates four anti-MSLN TFP T cell product doses: 5×10⁷/m², 1×10⁸/m², 5×10⁸/m², and 1×10⁹/m². At each dose, the anti-MSLN TFP T cell product is first administered alone and, if deemed safe, is then administered following lymphodepletion with fludarabine and cyclophosphamide. For the purpose of dose escalation, the addition of lymphodepletion is considered a higher dose level even when using the same anti-MSLN TFP T cell product dose. At each dose level, a dose range of ±15% of the target dose may be administered.

The anti-MSLN TFP T cell product is supplied cryopreserved in cryostorage bags. The product in the bag is opaque, with cream to white color. The cryostorage bags containing anti-MSLN TFP T cell product arrive frozen in a liquid nitrogen dry shipper. The bags are stored in vapor phase of liquid nitrogen and the product remains frozen until the subject is ready for treatment to assure viable live autologous cells are administered to the subject. Several inactive ingredients are added to the product to assure viability and stability of the live cells through the freezing, thawing, and infusion process. Each bag contains a subject specific product, and the intended subject is identified by subject ID number. The product is thawed and administered to the subject as specified in the investigational product manual. The product must not be thawed until the subject is ready for the infusion. In case of accidental overdose, treatment is supportive. Corticosteroid therapy and/or tocilizumab may be considered if any dose is associated with severe toxicity.

### Example 10: Method of Anti-MSLN TFP Infusion

On day 0 of the study, subjects participating in the phase 1 portion of the study receive anti-MSLN TFP T cell product within the dose range of 5×10⁷ to 1×10⁹ transduced cells/square meter of surface area (depending on the dose level) by intravenous infusion. The recommended dose for subjects participating in the phase 2 portion is determined at the end of the dose escalating phase 1.

The anti-MSLN TFP T cell product is a subject-specific product. Upon receipt, verification that the product and subject-specific labels match the specific subject information is essential. The product is not infused if the information on the subject-specific label does not match the intended subject. Prior to infusion, 2 clinical personnel in the presence of the subject independently verify and confirm that the information on the infusion bag label is correctly matched to the subject, in accordance with institutional practice for the administration of cell products.

Thirty to 60 minutes prior to cell infusion, subjects are premedicated against potential infusion reactions with antihistamines and acetaminophen (paracetamol) as per institutional practice. Steroids are not administered as premedication for T cell infusion due to their lymphotoxic potential against the anti-MSLN TFP T cell product.

Anti-MSLN TFP T cells must not be thawed until immediately prior to infusion. The product can be thawed either in a water bath at the subject's bedside or with a device such as a GE ViaThaw in a centralized facility, according to Institutional standard procedures. The cells are infused without delay and, if thawed centrally, are transported to the subject by appropriately trained clinical staff, to preserve the chain of custody. The anti-MSLN TFP T cell product is not washed or otherwise processed.

It is expected that the infusion will commence within approximately 10 minutes of thawing (or within 10 minutes of receipt if thawed centrally) and complete within 45 minutes of thawing (or receipt from centralized thawing facility) to minimize exposure of the anti-MSLN TFP T cell product to cryoprotectant.

The anti-MSLN TFP T cell product is administered using a dual spike infusion set by gravity over 15 to 30 minutes (in the absence of reaction) via non-filtered tubing. The bag is be gently agitated during infusion to avoid cell clumping. Infusion pumps must not be used.

For administration of the anti-MSLN TFP T cells, 100 to 250 ml of 0.9% NaCl is connected to the second lumen of the infusion set, used to prime the line, and then the lumen closed. On completion of the infusion of a bag of anti-MSLN TFP T cells, the main line is closed and approximately 50 ml NaCl transferred into the cell bag, and then infused to minimize cell loss. This process is repeated for each cell bag if multiple bags are provided.

On completion of the cell infusion the set is flushed using additional saline from the attached bag. In the event that Institutional practice requires a single spike infusion set, the line is flushed with 0.9% NaCl once the infusion is complete. In the event of adverse reaction to anti-MSLN TFP T cell infusion, the infusion rate is reduced, and the reaction managed according to Institutional standard procedures.

Vital signs are recorded within 10 minutes prior to the infusion and at 5, 15, and 30 minutes and at 1, 1.5, 2, and 4 hours after the infusion has started. For the phase 1 portion of the study, subjects are admitted for inpatient observation overnight following the infusion of anti-MSLN TFP T cells.

### Example 11: Toxicity Management

### Infection Prophylaxis

Subjects receive prophylaxis for infection with pneumocystis Jiroveci pneumonia, herpes virus, varicella zoster, and fungal infections according to National Comprehensive Cancer Network guidelines or standard Institutional practice.

The instructions for prophylaxis are as follows:
Pneumocystis Jiroveci pneumonia: daily single strength trimethoprim sulfamethoxazole, for one year.
Herpes Simplex and Varicella Zoster: acyclovir (800 mg twice daily) or valacyclovir (500 mg twice daily) for one year.
CMV: patients are screened for CMV seropositivity at Baseline. If CMV viremia is detected at Baseline, treatment is initiated prior to lymphodepleting chemotherapy. Subjects are monitored for CMV as detailed in the Schedule of Events. If CMV viremia is documented, an infectious disease specialist is consulted and treatment initiated (if required) according to Institutional practice. Recommended regimens include ganciclovir-based therapy if ANC≥1.0×10⁹/L, and foscarnet if ANC<1.0×10⁹/L.

### Tumor Lysis Syndrome

All subjects with significant tumor burden and without a contraindication receive tumor lysis syndrome prophylaxis (e.g., allopurinol) as per institutional guidelines prior to anti-MSLN TFP T cell infusion. Prophylaxis is discontinued when the risk of tumor lysis has passed.

### Cytokine Release Syndrome

Cytokine release syndrome has been described with therapies that activate T lymphocytes such as BiTEs (e.g., blinatumomab) and ACT (e.g., CART cell therapy). CRS results from the massive release of cytokines from cells targeted by therapeutics, immune effector cells recruited to the tumor area and subject's immune cells activated during this process. CRS is associated with a variety of clinical signs and symptoms, which can be life-threatening, including cardiac, gastrointestinal, laboratory (disseminated intravascular coagulation, renal and hepatic abnormalities), neurological, respiratory, skin, vascular (hypotension), and constitutional (fever, rigors, headaches malaise, fatigue arthralgia, nausea and vomiting). The goal of CRS management is to prevent life-threatening conditions while preserving the potential benefit of anti-MSLN TFP T cell-induced antitumor activity. A revised CRS severity grading system was published by NCI investigators and is highlighted below (Table 3).

TABLE 3
Cytokine Release Syndrome Grading Scale

| | |
|---|---|
| Grade 1 | Symptoms are not life threatening and require |
| | symptomatic treatment only (e.g., fever, nausea, fatigue, |
| | headache, myalgia, malaise). |
| Grade 2 | Symptoms require and respond to moderate |
| | intervention oxygen requirement < 40% or |
| | hypotension responsive to fluids or low dose of one |
| | vasopressor or grade 2 organ toxicity. |
| Grade 3 | Symptoms require and respond to aggressive |
| | intervention oxygen requirement ≥ 40% or |
| | hypotension requiring high dose or multiple vasopressors |
| | or grade 3 organ toxicity or grade 4 transaminitis. |
| Grade 4 | Life-threatening symptoms, requirements for |
| | ventilator support or, grade 4 organ toxicity (excluding |
| | transaminitis) |
| Grade 5 | Death |

Source: Lee et. al., 2014

A recommended CRS management algorithm according to grade is illustrated in Table 4 below. The algorithm has been further adapted from Common Terminology Criteria for Adverse Events (CTCAE) for use with immunotherapy and is implemented in accordance with Institutional guidelines. The diagnosis of CRS is clinical and it is supported by the exclusion of infection as well as the presence of increased cytokine levels and other biomarkers. If CRS is suspected, in addition to assessment for infection, cytokine and C-Reactive Protein (CRP) levels are measured approximately every other day until symptoms are improving or an alternative diagnosis is confirmed.

TABLE 4
Management Guidelines for Cytokine Release Syndrome

| | Clinical Presentation for Grading | |
|---|---|---|
| Grade | Assessment | Management Guidelines |
| 1 | Constitutional symptoms not life- | Vigilant supportive care^{a} |
| | threatening (e.g., fever, nausea, | Assess for infection and treat^{b} |
| | fatigue, headache, myalgia, malaise) | |
| 2 | Symptoms require and respond to | Monitor cardiac and other organ function |
| | moderate intervention (hypotension | Vigilant supportive care^{a} |
| | responds to fluids or one low dose | Assess for infection and treat^{b} |
| | pressor, hypoxia responds to | Treat hypotension with fluid and pressors |
| | <40% O₂, and/or grade 2 organ | Administer O₂ for hypoxia |
| | toxicity) | Consider tocilizumab 8 mg/kg iv (maximum |
| | | 800 mg) ± corticosteroids^{c} in subjects with |
| | | extensive co-morbidities or of older age |
| 3 | Symptoms require and respond to | Monitor subject very closely for cardiac and other |
| | aggressive intervention (hypotension | organ dysfunction. Most likely will require |
| | requires multiple pressors or high-dose | monitoring in an intensive care unit (ICU). |
| | pressors, hypoxia requires □ 40% O₂, | Vigilant supportive care^{a} |
| | grade 3 organ toxicity or grade 4 | Assess for infection and treat^{b} |
| | transaminitis) | Treat hypotension with fluid and pressors. |
| | | Administer O₂ for hypoxia. |
| | | Administer tozilizumab 8 mg/kg iv (maximum |
| | | 800 mg) ± corticosteroids^{c} |
| 4 | Life-threatening symptoms | Manage subject in ICU |
| | Grade 4 organ toxicity (excluding | Intensive supportive care including mechanical |
| | transaminitis) | ventilation, fluids, pressors, antibiotics, and other |
| | | measures as required |
| | | Administer tocilizumab 8 mg/kg iv (maximum |
| | | 800 mg) ± corticosteroids^{c} |
| 5 | Death | Not applicable. |

iv = intravenous(ly); IL-1R = interleukin 1R; TNF-α = tumor necrosis factor alpha.
^{a}= Supportive care includes monitor fluid balance, maintain adequate hydration, and blood pressure
^{b}= Assessment and treatment to include history and physical, blood, and urine cultures, imaging studies, administration of antimicrobial agents for concurrent bacterial infections, and for treatment of fever and neutropenia as per institutional practice; and antipyretics, analgesics as needed.
^{c}= Corticosteroid dose: 2 mg/kg methylprednisolone as an initial dose, then 2 mg/kg per day (plan for rapid taper). Other immunosuppressor agents may be used, including TNF-α and IL-1R inhibitors.

Subjects with severe CRS may receive tocilizumab, corticosteroids, or both. Tocilizumab is a humanized anti-IL-6 receptor antibody that has been approved by US FDA for the management of CRS. Subjects may receive a repeat dose if clinical signs and symptoms do not improve within 24 to 48 hours. Subjects are considered responders if CRS resolves within 14 days of the first dose of tocilizumab, no more than 2 doses of tocilizumab are needed, and no drugs other than tocilizumab and corticosteroids are used for treatment.

Side effects attributed to chronic use of tocilizumab in rheumatologic disease include transaminitis, thrombocytopenia, elevated cholesterol and low-density lipoproteins, neutropenia, and increased infections, but acute infusional toxicities have not been reported in CRS use.

### Fever and Neutropenia

Evaluation for a source of infection is performed per institutional guidelines. Fever is treated with acetaminophen and comfort measures. Corticosteroids is avoided. Subjects who are neutropenic and febrile should receive broad-spectrum antibiotics.

Maintenance IV fluids (normal saline) is recommended in subjects with high fevers, particularly if oral intake is poor and/or if the subject has tachycardia.

Neutropenia is a common effect of lymphodepleting chemotherapy. Prophylactic G-CSF (e.g., filgrastim) is recommended in all subjects for management of neutropenia according to published guidelines (e.g., ASCO guidelines). G-CSF may be started 24 hours after the administration of lymphodepleting chemotherapy and continued until neutrophil recovery according to institutional practice. Long-acting (pegylated) G-CSF may be used as per institutional standard practice. Pegylated G-CSF is given as 1 dose 24 hours post the final dose of cyclophosphamide. GM-CSF is not allowed to be used while on study.

### Blood Product Support

The subject should receive platelets and packed red blood cells as needed as per institutional guidelines. All blood products is irradiated. Hemoglobin is kept >8.0 gm/dL and platelets >20,000/mm³. Leukocyte filters is utilized for all blood and platelet transfusions to decrease sensitization to transfused white blood cells and decrease the risk of cytomegalovirus (CMV) infection.

### Neurotoxicity

Neurotoxicity (e.g., encephalopathy, somnolence, delirium, seizures, aphasia) has been observed with different ACT modalities, particularly with anti-CD19 CAR T cell therapies, which has been termed CAR T cell-related encephalopathy syndrome (Table 4). Evaluation of any new onset neurotoxicity should include a neurological examination (e.g., CARTOX-10), brain MRI, papilledema grading, and examination of the cerebrospinal fluid (CSF) as clinically indicated.

TABLE 5
Grading of Chimeric Antigen Receptor T cell-related Encephalopathy Syndrome

| Symptom or Sign | Grade 1 | Grade 2 | Grade 3 | Grade 4 |
|---|---|---|---|---|
| Neurological | 7-9 (mild | 3-6 (moderate | 0-2 (severe impairment) | Subject in critical |
| assessment score | impairment) | impairment) | | condition, and/or |
| (by CARTOX-10*) | | | | obtunded and cannot |
| | | | | perform assessment of |
| | | | | tasks |
| Raised intracranial | NA | NA | Stage 1-2 papilledema‡, | Stage 3-5 papilledema^{a}, |
| pressure | | | or CSF opening | or CSF opening pressure ≥ |
| | | | pressure < 20 mmHg | 20 mmHg, or cerebral |
| | | | | edema |
| Seizures or motor | NA | NA | Partial seizure, or non- | Generalized seizures, or |
| weakness | | | convulsive seizures on | convulsive or non- |
| | | | EEG with response to | convulsive status |
| | | | benzodiazepine | epilepticus, or new motor |
| | | | | weakness |

CARTOX-10 = CAR-T cell-therapy-associated toxicity 10-point neurological assessment; CSF = cerebrospinal fluid; EEG = electroencephalogram; NA = not applicable.
^{a}= Papilledema grading is performed according to the modified Frisén scale. Source: Neepalu et al, 2018; Frisen, 1982

Endotracheal intubation may be needed for airway protection in severe cases. Corticosteroids may be considered for any severe or life-threatening neurotoxicity and anti-seizure and sedatives may be considered as clinically indicated. Guidance for management of chimeric antigen receptor T cell-related encephalopathy syndrome is provided in Table 6.

TABLE 6
Management of Chimeric Antigen Receptor T cell-related Encephalopathy Syndrome

Grade 1
Vigilant supportive care; aspiration precautions; intravenous (iv) hydration
Withhold oral intake of food, medicines, and fluids, and assess swallowing
Convert all oral medications and/or nutrition to iv if swallowing is impaired
Avoid medications that cause central nervous system depression
Low doses of lorazepam (0.25-0.5 mg iv every 8 h) or haloperidol (0.5 mg iv every 6 h) can be used,
with careful monitoring, for agitated subjects
Neurology consultation
Fundoscopic exam to assess for papilledema
MRI of the brain with and without contrast; diagnostic lumbar puncture with measurement of opening
pressure; MRI spine if the subject has focal peripheral neurological deficits; CT scan of the brain can be
performed if MRI of the brain is not feasible
Daily 30 min electroencephalogram (EEG) until toxicity symptoms resolve; if no seizures are detected
on EEG, continue levetiracetam 750 mg every 12 h
If EEG shows non-convulsive status epilepticus, treat as per algorithm in BOX 3
Consider anti-IL-6 therapy with tocilizumab 8 mg/kg iv (maximum 800 mg) or siltuximab 11 mg/kg iv, if
CRES is associated with concurrent cytokine-release syndrome (CRS)
Grade 2
Supportive care and neurological work-up as described for grade 1 CRES
Tocilizumab 8 mg/kg (maximum 800 mg) iv or siltuximab 11 mg/kg iv if associated with concurrent
CRS
Dexamethasone 10 mg iv every 6 h or methylprednisolone 1 mg/kg iv every 12 h if refractory to anti-
IL-6 therapy, or for CRES without concurrent CRS
Consider transferring subject to intensive-care unit (ICU) if CRES associated with grade ≥ 2 CRS
Grade 3
Supportive care and neurological work-up as indicated for grade 1 CRES
ICU transfer is recommended
Anti-IL-6 therapy if associated with concurrent CRS, as described for grade 2 CRES and if not
administered previously
Corticosteroids as outlined for grade 2 CRES if symptoms worsen despite anti-IL-6 therapy, or for
CRES without concurrent CRS; continue corticosteroids until improvement to grade 1 CRES and then
taper
Stage 1 or 2 papilledema with cerebrospinal fluid (CSF) opening pressure < 20 mmHg is treated as per
algorithm described in Neelapu, et al, 2018
Consider repeat neuroimaging (CT or MRI) every 2-3 days if subject has persistent grade ≥ 3 CRES
Grade 4
Supportive care and neurological work-up as outlined for grade 1 CRES
ICU monitoring; consider mechanical ventilation for airway protection
Anti-IL-6 therapy and repeat neuroimaging as described for grade 3 CRES
High-dose corticosteroids continued until improvement to grade 1 CRES and then taper; for example,
methylprednisolone iv 1 g/day for 3 days, followed by rapid taper at 250 mg every 12 h for 2 days, 125
mg every 12 h for 2 days, and 60 mg every 12 h for 2 days
For convulsive status epilepticus, treat as per algorithm described in Neelapu, et al, 2018
Stage ≥ 3 papilledema, with a CSF opening pressure ≥ 20 mmHg or cerebral edema, is treated as per
algorithm in Neelapu, et al, 2018
CRES = Cell-related Encephalopathy Syndrome; CRS = Cytokine Release Syndrome; CT = computed tomography; IL-6 = interleukin 6; MRI = magnetic resonance imaging.

### Example 12: Study Procedures and Schedule of Events

### Mesothelin Screening

Only subjects with tumor mesothelin expression above the cut-off (□50% of cells that are 2+ and/or 3+) as determined by immunohistochemistry (IHC) at the central laboratory are eligible to receive anti-MSLN TFP T cell therapy. All subjects are tested for this expression level prior to moving forward with full screening.

A new excisional fresh tumor biopsy is required for determination of mesothelin expression, unless otherwise contraindicated (e.g., tumor not accessible). This fresh tumor biopsy is obtained at pre-screening and/or at some point prior to the baseline visit (visit 4). If a new biopsy cannot be obtained at the time of pre-screening visit, an archival biopsy is submitted, provided the tissue was obtained within the previous 12 months and that there is sufficient tissue for analysis of mesothelin expression. Mesothelin expression above the aforementioned cut-off on archival tissue enables proceeding with leukapheresis and shipment of the leukapheresis product to the anti-MSLN TFP T cell manufacturing facility (provided all other eligibility criteria are met). Nevertheless, a new tissue biopsy for fresh sample must still be obtained and mesothelin expression analysis results are available to the Sponsor at the time of the baseline visit (visit 4) in order to proceed with study treatment (i.e., lymphodepleting chemotherapy and/or anti-MSLN TFP T cell infusion). A fresh tissue biopsy is not required prior to baseline if the prescreening fresh sample has enough residual tissue to perform the required testing according to the central laboratory specifications. If an archival specimen is unavailable and the subject cannot undergo a new biopsy, the subject is declared ineligible. The subject's tumor biopsy is tested for mesothelin antigen xpression by IHC using an analytically validated assay at a certified central laboratory.

In addition, a secondary objective of the study is the development and validation of an IVD assay for the screening of tumor mesothelin expression for regulatory approval. In order to perform the mesothelin expression assay for study eligibility determination, and to complete the precision testing requirements for a regulatory approved companion diagnostic test, it is essential that sufficient amount of tumor tissue be submitted.

### Example 13: Clinical Assessments and Procedures

Demographics: Demographic data including year of birth, age, sex, race, and ethnicity is collected at prescreening.

Medical History: Relevant medical history is recorded at screening in the subject's medical record and CRF.

Disease history: The following information is recorded in the CRF: cancer diagnosis, date of initial diagnosis, location of disease at initial diagnosis, stage at initial diagnosis, type of histology, histological grade, results of any historical molecular testing performed (if available), date of diagnosis of metastatic disease, location of metastatic disease, stage at screening.

Subjects undergo a physical examination at screening and baseline visits and subsequently as specified in the SOE (Appendix A).

Vital Signs: Measurement of vital signs (temperature, pulse, respiratory rate, blood pressure, weight, and height) are made at screening and at baseline (excluding height).

Performance Status: Performance status is measured using the ECOG performance scale (Table 7).

TABLE 7
Eastern Cooperative Oncology Group Performance Status

| Grade | ECOG Performance Status |
|---|---|
| 0 | Fully active, able to carry on all pre-disease performance |
| | without restriction |
| 1 | Restricted in physically strenuous activity but ambulatory |
| | and able to carry out work of a light or sedentary nature, |
| | e.g., light house work, office work |
| 2 | Ambulatory and capable of all selfcare but unable to |
| | carry out any work activities; up and about more than |
| | 50% of waking hours |
| 3 | Capable of only limited selfcare; confined to bed or chair |
| | more than 50% of waking hours |
| 4 | Completely disabled; cannot carry on any selfcare; totally |
| | confined to bed or chair |
| 5 | Dead |

ECOG = Eastern Cooperative Oncology Group.
Source: Oken et al, 1982

Clinical Safety Assessments: Subjects are assessed for AEs and graded according to the National Cancer Institute (NCI) CTCAE Version 4.0. All AEs are recorded in the CRF.

Laboratory Assessments: The following laboratory assessments are performed by a certified laboratory designated by the sponsor. Local testing may be performed in addition to the central laboratory collection for the following samples: Hematology, Clinical chemistry, CRP, Uric acid, Lipase, Coagulation, Thyroid function tests, Infectious disease screening, CMV viremia monitoring, Urinalysis, Glomerular filtration rate, FCBP must have a negative urine or serum pregnancy test at screening and prior to starting lymphodepleting chemotherapy.

Cardiac Assessments: The following assessments are conducted in order to monitor subject safety: An echocardiogram or multiple-gated acquisition (MUGA) scan is performed at screening to determine eligibility as well as in week 2 post infusion. Additional assessments may be performed if clinically indicated. The same method of cardiac evaluation is used consistently for any follow-up assessments. Blood troponin levels are tested on the days when echocardiogram/MUGA scans are performed. ECGs: ECGs (12-lead) are performed at baseline visit (visit 4) following a minimum of 15 minutes of supine rest. A subject with tumor lesions abutting the pericardium undergoes continuous cardiac monitoring (telemetry) for 5 days post-infusion.

Tumor Response Assessments: Imaging scans of the chest, abdomen, and pelvis are performed at eligibility, baseline, week 4, week 8, week 12, week 24, and every 3 months until confirmed disease progression, study completion, or withdrawal. Acceptable imaging modalities for this study include:
Diagnostic-quality CT scan with oral and/or iv iodinated contrast of the chest and abdomen/pelvis (CT is the preferred modality for tumor assessments).
MRI of the abdomen/pelvis acquired before and after gadolinium contrast agent administration and a non-contrast enhanced CT of the chest, if a subject is contraindicated for contrast enhanced CT.

The same imaging modality and image-acquisition protocol (including the use of iv contrast) are consistently used across all time points for individual subjects to allow uniform comparison of lesions. Prior to starting the study, a detailed imaging acquisition protocol is provided to the sites to describe the requirements for image acquisition and the standardized procedure for the transfer of image data to a central vendor.

Tumor assessments are evaluated according to the RECIST v 1.1. To allow time for an immune response to become apparent and to account for potential post-treatment transient inflammation of the tumor site (‘pseudoprogression’), response assessments are not carried out before 4 weeks post anti-MSLN TFP, unless there is unequivocal clinical evidence of deterioration. If disease progression is equivocal, confirmation of disease progression is required by a follow-up scan performed at least 4 weeks apart, unless there is an immediate medical need to initiate anti-cancer therapy before the confirmatory scan can be performed. Disease progression is not declared until results from the confirmatory scan are available. If confirmed, the date of progression is that of the initial scan where progression was first suspected (ie, not the confirmatory scan).

For clinical decision making, investigators assess tumor response according to RECISTv 1.1. To the extent that it is feasible, local tumor assessments are performed by the same radiologist. Additionally, scans are submitted to a central imaging facility for independent read and to give a RESIST criteria response. Results from both reads are summarized and reconciled at the end of the study phase.

For subjects who have new lesions, response by RECIST (Nishino, 2013) is assessed by the investigator for exploratory purposes. For new lesions, information on whether the lesion is measurable or non-measurable is recorded in the CRF.

Period of Hospitalization for Lymphodepletion and/or anti-MSLN TFP Infusion: Both the lymphodepleting chemotherapy as well as the anti-MSLN TFP infusion are either given as an outpatient treatment or subjects may be hospitalized at the discretion of the Investigator. However, for the phase 1 portion of the study, subjects are admitted for inpatient observation overnight following anti-MSLN TFP infusion. Should a subject require hospitalization for administration of lymphodepleting chemotherapy, information of such hospitalization (e.g., reason, number of days) is recorded in the CRF.

### Example 14: Cytokine and Anti-MSLN TFP T Cell Antibody Analysis

Serum is collected at baseline, and at each visit post infusion up to 8 weeks, to allow for measurement of cytokines in the blood. Serum is also collected from subjects with suspected CRS with samples being taken approximately every other day until symptoms are improving or an alternative diagnosis is confirmed. Cytokines, growth factors and soluble receptors including but not limited to IL-1 β, IL-6, IFN-γ, TNF-α, IL-2, IL-8, IL-10, IL-12, IL-13, 15, and GM-CSF are measured utilizing a multiplexed assay. Measurement of the cytokine subset IL-1β, IL-6, IL-10, TNF-α, and IFN-γ is performed following Good Laboratory Practice procedures. All other measurements are performed as exploratory. Serum samples are also used to detect presence of antibodies against anti-MSLN TFP pre-infusion and at week 8. For serum samples that demonstrate increased anti-anti-MSLN TFP human antibodies at the week 8 visit over pre-infusion values, an attempt is made to obtain and test additional serum samples at 3-month intervals until the antibody levels return to baseline (or become negative) or up to 1 year from date of anti-MSLN TFP infusion, whichever occurs first.

### Example 15: Tumor Biopsies

The activity of anti-MSLN TFP T cells is impacted by other cellular elements within the tumor microenvironment (e.g., regulatory T cells). Evaluating the “immune landscape” within the tumor is critical for optimizing cancer immunotherapy. For this reason, core needle biopsies are requested at screening and/or baseline (to evaluate the immune status of the tumor before T cell infusion), week 8 (±2 weeks), at the expected time of an active anti-tumor response by infused T cells) and after disease progression is confirmed, unless tumor is not safely accessible. When possible, biopsies should consist of multiple cores taken from more than one lesion. In cases where tumor lesions are not be amenable to core needle biopsy, fine needle aspirates may be obtained based on interventional radiology recommendations.

While fresh tissue is preferable for the screening biopsy assessment, archival tissue may be used provided the biopsy was taken in the 12 months prior to screening. If fresh tissue is provided for screening purposes and remaining tissue is sufficient for research studies, the baseline biopsy can be omitted. Otherwise the baseline biopsy material may be collected anytime between 2 months and 1 week prior to the start of lymphodepleting chemotherapy, favoring a time point closer to the time of anti-MSLN TFP infusion. Tumor tissue is either taken from non-target lesions or from target lesions >2 cm. Every attempt is made to obtain biopsies at both screening and subsequent time points from the same lesion(s). The radiological (or clinical) status of the lesion(s) biopsied is noted at the time (e.g., decreased, stable, increased size or activity).

Biopsy material is collected after confirmation of disease progression, ideally on lesions that have progressed and on new lesions, to address mechanisms of resistance and to determine eligibility for re-treatment.

In subjects who have a serosal effusion, should there be a clinical requirement for removal of the effusion fluid at any time during study, samples are requested to be collected for the conduction of research studies. When available, serosal effusion fluid is collected in addition to, and not instead of the requested tumor biopsies, with the exception of MPM cases where tumor biopsy is not accessible and/or available.

Serosal effusion specimens are used to interrogate the tumor microenvironment prior to and after anti-MSLN TFP infusion to address mechanisms of sensitivity or resistance to therapy as well as kinetics of tumor clearance.

Tumor tissue is collected for path review, mesothelin expression, gene and/or protein expression profiling, and analysis of DNA alterations. Remaining tumor samples are stored for future exploratory analysis of DNA, RNA, or protein markers.

### Endnotes

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure described herein may be employed in practicing the disclosure. It is intended that the following claims define the scope of the present disclosure and that methods and structures within the scope of these claims and their equivalents be covered thereby.

APPENDIX A
SEQUENCE SUMMARY

| SEQ | | |
|---|---|---|
| ID | | |
| NO. | Name | Sequence |
| 1 | Short | GGGGSGGGGSGGGGSLE |
| | Linker 1 | |
| 2 | Short | AAAGGGGSGGGGSGGGGSLE |
| | Linker 2 | |
| 3 | Long | AAAIEVMYPPPYLGGGGSGGGGSGGGGSLE |
| | Linker | |
| 4 | human | MQSGTHWRVLGLCLLSVGVWGQDGNEEMGGITQTPYKVSISGT |
| | CD3-ϵ | TVILTCPQYPGSEILWQHNDKNIGGDEDDKNIGSDEDHLSLKEFSE |
| | | LEQSGYYVCYPRGSKPEDANFYLYLRARVCENCMEMDVMSVAT |
| | | IVIVDICITGGLLLLVYYWSKNRKAKAKPVTRGAGAGGRQRGQN |
| | | KERPPPVPNPDYEPIRKGQRDLYSGLNQRRI |
| 5 | human | MEQGKGLAVLILAIILLQGTLAQSIKGNHLVKVYDYQEDGSVLLT |
| | CD3-γ | CDAEAKNITWFKDGKMIGFLTEDKKKWNLGSNAKDPRGMYQC |
| | | KGSQNKSKPLQVYYRMCQNCIELNAATISGFLFAEIVSIFVLAVG |
| | | VYFIAGQDGVRQSRASDKQTLLPNDQLYQPLKDREDDQYSHLQG |
| | | NQLRRN |
| 6 | human | MEHSTFLSGLVLATLLSQVSPFKIPIEELEDRVFVNCNTSITWVEG |
| | CD3-δ | TVGTLLSDITRLDLGKRILDPRGIYRCNGTDIYKDKESTVQVHYR |
| | | MCQSCVELDPATVAGIIVTDVIATLLLALGVFCFAGHETGRLSGA |
| | | ADTQALLRNDQVYQPLRDRDDAQYSHLGGNWARNKS |
| 7 | human | MKWKALFTAAILQAQLPITEAQSFGLLDPKLCYLLDGILFIYGVIL |
| | CD3-ζ | TALFLRVKFSRSADAPAYQQGQNQLYNELNLGRREEYDVLDKRR |
| | | GRDPEMGGKPQRRKNPQEGLYNELQKDKMAEAYSEIGMKGERR |
| | | RGKGHDGLYQGLSTATKDTYDALHMQALPPR |
| 8 | human | MAGTWLLLLLALGCPALPTGVGGTPFPSLAPPIMLLVDGKQQMV |
| | TCR α- | VVCLVLDVAPPGLDSPIWFSAGNGSALDAFTYGPSPATDGTWTN |
| | chain | LAHLSLPSEELASWEPLVCHTGPGAEGHSRSTQPMHLSGEASTAR |
| | | TCPQEPLRGTPGGALWLGVLRLLLFKLLLFDLLLTCSCLCDPAGP |
| | | LPSPATTTRLRALGSHRLHPATETGGREATSSPRPQPRDRRWGDT |
| | | PPGRKPGSPVWGEGSYLSSYPTCPAQAWCSRSALRAPSSSLGAFF |
| | | AGDLPPPLQAGA |
| 9 | human | PNIQNPDPAVYQLRDSKSSDKSVCLFTDFDSQTNVSQSKDSDVYI |
| | TCR α- | TDKTVLDMRSMDFKSNSAVAWSNKSDFACANAFNNSIIPEDTFFP |
| | chain C | SPESSCDVKLVEKSFETDTNLNFQNLSVIGFRILLLKVAGFNLLMT |
| | region | LRLWSS |
| 10 | human | MAMLLGASVLILWLQPDWVNSQQKNDDQQVKQNSPSLSVQEGR |
| | TCR α- | ISILNCDYTNSMFDYFLWYKKYPAEGPTFLISISSIKDKNEDGRFT |
| | chain V | VFLNKSAKHLSLHIVPSQPGDSAVYFCAAKGAGTASKLTFGTGTR |
| | region | LQVTL |
| | CTL-L17 | |
| 11 | human | EDLNKVFPPEVAVFEPSEAEISHTQKATLVCLATGFFPDHVELSW |
| | TCR β- | WVNGKEVHSGVSTDPQPLKEQPALNDSRYCLSSRLRVSATFWQN |
| | chain C | PRNHFRCQVQFYGLSENDEWTQDRAKPVTQIVSAEAWGRADCG |
| | region | FTSVSYQQGVLSATILYEILLGKATLYAVLVSALVLMAMVKRKD |
| | | F |
| 12 | human | MGTSLLCWMALCLLGADHADTGVSQNPRHNITKRGQNVTFRCD |
| | TCR β- | PISEHNRLYWYRQTLGQGPEFLTYFQNEAQLEKSRLLSDRFSAER |
| | chain V | PKGSFSTLEIQRTEQGDSAMYLCASSLAGLNQPQHFGDGTRLSIL |
| | region | |
| | CTL-L17 | |
| 13 | human | MDSWTFCCVSLCILVAKHTDAGVIQSPRHEVTEMGQEVTLRCKPI |
| | TCR β- | SGHNSLFWYRQTMMRGLELLIYFNNNVPIDDSGMPEDRFSAKMP |
| | chain V | NASFSTLKIQPSEPRDSAVYFCASSFSTCSANYGYTFGSGTRLTVV |
| | region | |
| | YT35 | |
| 14 | MSLN | acgcgtgtagtcttatgcaatactcttgtagtcttgcaacatggtaacgatgagttagcaacatgccttaca |
| | DNA | aggagagaaaaagcaccgtgcatgccgattggtggaagtaaggtggtacgatcgtgccttattaggaa |
| | Seq. | ggcaacagacgggtctgacatggattggacgaaccactgaattgccgcattgcagagatattgtatttaa |
| | | gtgcctagctcgatacaataaacgggtctctctggttagaccagatctgagcctgggagctctctggcta |
| | | actagggaacccactgcttaagcctcaataaagcttgccttgagtgcttcaagtagtgtgtgcccgtctgtt |
| | | gtgtgactctggtaactagagatccctcagacccttttagtcagtgtggaaaatctctagcagtggcgccc |
| | | gaacagggacctgaaagcgaaagggaaaccagagctctctcgacgcaggactcggcttgctgaagc |
| | | gcgcacggcaagaggcgaggggcggcgactggtgagtacgccaaaaattttgactagcggaggcta |
| | | gaaggagagagatgggtgcgagagcgtcagtattaagcgggggagaattagatcgcgatgggaaaa |
| | | aattcggttaaggccagggggaaagaaaaaatataaattaaaacatatagtatgggcaagcagggagct |
| | | agaacgattcgcagttaatcctggcctgttagaaacatcagaaggctgtagacaaatactgggacagcta |
| | | caaccatcccttcagacaggatcagaagaacttagatcattatataatacagtagcaaccctctattgtgtg |
| | | catcaaaggatagagataaaagacaccaaggaagctttagacaagatagaggaagagcaaaacaaaa |
| | | gtaagaccaccgcacagcaagcggccactgatcttcagacctggaggaggagatatgagggacaatt |
| | | ggagaagtgaattatataaatataaagtagtaaaaattgaaccattaggagtagcacccaccaaggcaaa |
| | | gagaagagtggtgcagagagaaaaaagagcagtgggaataggagctttgttccttgggttcttgggag |
| | | cagcaggaagcactatgggcgcagcctcaatgacgctgacggtacaggccagacaattattgtctggt |
| | | atagtgcagcagcagaacaatttgctgagggctattgaggcgcaacagcatctgttgcaactcacagtct |
| | | ggggcatcaagcagctccaggcaagaatcctggctgtggaaagatacctaaaggatcaacagctcctg |
| | | gggatttggggttgctctggaaaactcatttgcaccactgctgtgccttggaatgctagttggagtaataaa |
| | | tctctggaacagattggaatcacacgacctggatggagtgggacagagaaattaacaattacacaagctt |
| | | aatacactccttaattgaagaatcgcaaaaccagcaagaaaagaatgaacaagaattattggaattagat |
| | | aaatgggcaagtttgtggaattggtttaacataacaaattggctgtggtatataaaattattcataatgatagt |
| | | aggaggcttggtaggtttaagaatagtttttgctgtactttctatagtgaatagagttaggcagggatattca |
| | | ccattatcgtttcagacccacctcccaaccccgaggggacccgacaggcccgaaggaatagaagaag |
| | | aaggtggagagagagacagagacagatccattcgattagtgaacggatctcgacggtatcggttaactt |
| | | ttaaaagaaaaggggggattggggggtacagtgcaggggaaagaatagtagacataatagcaacaga |
| | | catacaaactaaagaattacaaaaacaaattacaaaattcaaaattttatcgatactagtattatgcccagta |
| | | catgaccttatgggactttcctacttggcagtacatctacgtattagtcatcgctattaccatggtgatgcgg |
| | | ttttggcagtacatcaatgggcgtggatagcggtttgactcacggggatttccaagtctccaccccattga |
| | | cgtcaatggga |
| | | gtttgttttggcaccaaaatcaacgggactttccaaaatgtcgtaacaactccgccccattgacgcaaatg |
| | | ggcggtaggcgtgtacggtgggaggtttatataagcagagctcgtttagtgaaccgtcagatcgcctgg |
| | | agacgccatccacgctgttttgacctccatagaagattctagagccgccaccatgcttctcctggtgacaa |
| | | gccttctgctctgtgagttaccacacccagcattcctcctgatcccagacattcagcaggtccagctccag |
| | | cagtctggccctgaactcgaaaaacctggcgctagcgtgaaaatttcctgtaaagcctccggctactcttt |
| | | tactggctacacaatgaattgggtgaaacagtctcacggcaaatccctcgaatggatcggactcatcaca |
| | | ccctacaatggcgcctcttcctacaaccagaaattccggggcaaggcaacactcactgtggacaaatca |
| | | tcctctaccgcctacatggatctgctctccctcacatctgaggactccgctgtctacttttgtgcccgagga |
| | | ggatacgacggacgaggattcgattactggggacagggaacaactgtgaccgtgtctagtggcggcg |
| | | gagggagtggaggcggaggatcttctggcgggggatccgatattgaactcacacagtctcccgctatc |
| | | atgtctgcttctcccggcgagaaagtgactatgacttgctctgcttcctcttctgtgtcctacatgcactggt |
| | | accagcagaaatctggcacatcccctaaacggtggatctacgatactagcaaactggcatccggcgtgc |
| | | ctgggcgattctctggctctggctctggcaactcttactctctcacaatctcatctgtcgaggctgaggacg |
| | | atgccacatactactgtcagcagtggtctaaacacccactcacattcggcgctggcactaaactggaaat |
| | | aaaagcggccgcaggtggcggcggttctggtggcggcggttctggtggcggcggttctctcgaggat |
| | | ggtaatgaagaaatgggtggtattacacagacaccatataaagtctccatctctggaaccacagtaatatt |
| | | gacatgccctcagtatcctggatctgaaatactatggcaacacaatgataaaaacataggcggtgatgag |
| | | gatgataaaaacataggcagtgatgaggatcacctgtcactgaaggaattttcagaattggagcaaagtg |
| | | gttattatgtctgctaccccagaggaagcaaaccagaagatgcgaacttttatctctacctgagggcaag |
| | | agtgtgtgagaactgcatggagatggatgtgatgtcggtggccacaattgtcatagtggacatctgcatc |
| | | actgggggcttgctgctgctggtttactactggagcaagaatagaaaggccaaggccaagcctgtgaca |
| | | cgaggagcgggtgctggcggcaggcaaaggggacaaaacaaggagaggccaccacctgttcccaa |
| | | cccagactatgagcccatccggaaaggccagcgggacctgtattctggcctgaatcagagacgcatct |
| | | gataagaattcgatccgcggccgcgaaggatctgcgatcgctccggtgcccgtcagtgggcagagcg |
| | | cacatcgcccacagtccccgagaagttggggggaggggtcggcaattgaacgggtgcctagagaag |
| | | gtggcgcggggtaaactgggaaagtgatgtcgtgtactggctccgcctttttcccgagggtgggggag |
| | | aaccgtatataagtgcagtagtcgccgtgaacgttctttttcgcaacgggtttgccgccagaacacagctg |
| | | aagcttcgaggggctcgcatctctccttcacgcgcccgccgccctacctgaggccgccatccacgccg |
| | | gttgagtcgcgttctgccgcctcccgcctgtggtgcctcctgaactgcgtccgccgtctaggtaagtttaa |
| | | agctcaggtcgagaccgggcctttgtccggcgctcccttggagcctacctagactcagccggctctcca |
| | | cgctttgcctgaccctgcttgctcaactctacgtctttgtttcgttttctgttctgcgccgttacagatccaagc |
| | | tgtgaccggcgcctacgctagatgaccgagtacaagcccacggtgcgcctcgccacccgcgacgacg |
| | | tccccagggccgtacgcaccctcgccgccgcgttcgccgactaccccgccacgcgccacaccgtcga |
| | | tccggaccgccacatcgagcgggtcaccgagctgcaagaactcttcctcacgcgcgtcgggctcgac |
| | | atcggcaaggtgtgggtcgcggacgacggcgccgcggtggcggtctggaccacgccggagagcgt |
| | | cgaagcgggggcggtgttcgccgagatcggcccgcgcatggccgagttgagcggttcccggctggc |
| | | cgcgcagcaacagatggaaggcctcctggcgccgcaccggcccaaggagcccgcgtggttcctggc |
| | | caccgtcggcgtctcgcccgaccaccagggcaagggtctgggcagcgccgtcgtgctccccggagt |
| | | ggaggcggccgagcgcgccggggtgcccgccttcctggagacctccgcgccccgcaacctcccctt |
| | | ctacgagcggctcggcttcaccgtcaccgccgacgtcgaggtgcccgaaggaccgcgcacctggtgc |
| | | atgacccgcaagcccggtgcctgagtcgacaatcaacctctggattacaaaatttgtgaaagattgactg |
| | | gtattcttaactatgttgctccttttacgctatgtggatacgctgctttaatgcctttgtatcatgctattgcttcc |
| | | cgtatggctttcattttctcctccttgtataaatcctggttgctgtctctttatgaggagttgtggcccgttgtca |
| | | ggcaacgtggcgtggtgtgcactgtgtttgctgacgcaacccccactggttggggcattgccaccacct |
| | | gtcagctcctttccgggactttcgctttccccctccctattgccacggcggaactcatcgccgcctgccttg |
| | | cccgctgctggacaggggctcggctgttgggcactgacaattccgtggtgttgtcggggaaatcatcgt |
| | | cctttccttggctgctcgcctgtgttgccacctggattctgcgcgggacgtccttctgctacgtcccttcgg |
| | | ccctcaatccagcggaccttccttcccgcggcctgctgccggctctgcggcctcttccgcgtcttcgcctt |
| | | cgccctcagacgagtcggatctccctttgggccgcctccccgcctggtacctttaagaccaatgacttac |
| | | aaggcagctgtagatcttagccactttttaaaagaaaaggggggactggaagggctaattcactcccaa |
| | | cgaaaataagatctgctttttgcttgtactgggtctctctggttagaccagatctgagcctgggagctctctg |
| | | gctaactagggaacccactgcttaagcctcaataaagcttgccttgagtgcttcaagtagtgtgtgcccgt |
| | | ctgttgtgtgactctggtaactagagatccctcagacccttttagtcagtgtggaaaatctctagcagtagta |
| | | gttcatgtcatcttattattcagtatttataacttgcaaagaaatgaatatcagagagtgagaggaacttgttt |
| | | attgcagcttataatggttacaaataaagcaatagcatcacaaatttcacaaataaagcatttttttcactgca |
| | | ttctagttgtggtttgtccaaactcatcaatgtatcttatcatgtctggctctagctatcccgcccctaactccg |
| | | cccagttccgcccattctccgccccatggctgactaattttttttatttatgcagaggccgaggccgcctcg |
| | | gcctctgagctattccagaagtagtgaggaggcttttttggaggcctagacttttgcagagacggcccaa |
| | | attcgtaatcatggtcatagctgtttcctgtgtgaaattgttatccgctcacaattccacacaacatacgagc |
| | | cggaagcataaagtgtaaagcctggggtgcctaatgagtgagctaactcacattaattgcgttgcgctca |
| | | ctgcccgctttccagtcgggaaacctgtcgtgccagctgcattaatgaatcggccaacgcgcggggag |
| | | aggcggtttgcgtattgggcgctcttccgcttcctcgctcactgactcgctgcgctcggtcgttcggctgc |
| | | ggcgagcggtatcagctcactcaaaggcggtaatacggttatccacagaatcaggggataacgcagga |
| | | aagaacatgtgagcaaaaggccagcaaaaggccaggaaccgtaaaaaggccgcgttgctggcgttttt |
| | | ccataggctccgcccccctgacgagcatcacaaaaatcgacgctcaagtcagaggtggcgaaacccg |
| | | acaggactataaagataccaggcgtttccccctggaagctccctcgtgcgctctcctgttccgaccctgc |
| | | cgcttaccggatacctgtccgcctttctcccttcgggaagcgtggcgctttctcatagctcacgctgtaggt |
| | | atctcagttcggtgtaggtcgttcgctccaagctgggctgtgtgcacgaaccccccgttcagcccgaccg |
| | | ctgcgccttatccggtaactatcgtcttgagtccaacccggtaagacacgacttatcgccactggcagca |
| | | gccactggtaacaggattagcagagcgaggtatgtaggcggtgctacagagttcttgaagtggtggcct |
| | | aactacggctacactagaaggacagtatttggtatctgcgctctgctgaagccagttaccttcggaaaaa |
| | | gagttggtagctcttgatccggcaaacaaaccaccgctggtagcggtggtttttttgtttgcaagcagcag |
| | | attacgcgcagaaaaaaaggatctcaagaagatcctttgatcttttctacggggtctgacgctcagtggaa |
| | | cgaaaactcacgttaagggattttggtcatgagattatcaaaaaggatcttcacctagatccttttaaattaa |
| | | aaatgaagttttaaatcaatctaaagtatatatgagtaaacttggtctgacagttaccaatgcttaatcagtga |
| | | ggcacctatctcagcgatctgtctatttcgttcatccatagttgcctgactccccgtcgtgtagataactacg |
| | | atacgggagggcttaccatctggccccagtgctgcaatgataccgcgagacccacgctcaccggctcc |
| | | agatttatcagcaataaaccagccagccggaagggccgagcgcagaagtggtcctgcaactttatccg |
| | | cctccatccagtctattaattgttgccgggaagctagagtaagtagttcgccagttaatagtttgcgcaacg |
| | | ttgttgccattgctacaggcatcgtggtgtcacgctcgtcgtttggtatggcttcattcagctccggttccca |
| | | acgatcaaggcgagttacatgatcccccatgttgtgcaaaaaagcggttagctccttcggtcctccgatc |
| | | gttgtcagaagtaagttggccgcagtgttatcactcatggttatggcagcactgcataattctcttactgtca |
| | | tgccatccgtaagatgcttttctgtgactggtgagtactcaaccaagtcattctgagaatagtgtatgcggc |
| | | gaccgagttgctcttgcccggcgtcaatacgggataataccgcgccacatagcagaactttaaaagtgct |
| | | catcattggaaaacgttcttcggggcgaaaactctcaaggatcttaccgctgttgagatccagttcgatgt |
| | | aacccactcgtgcacccaactgatcttcagcatcttttactttcaccagcgtttctgggtgagcaaaaacag |
| | | gaaggcaaaatgccgcaaaaaagggaataagggcgacacggaaatgttgaatactcatactatcctttt |
| | | tcaatattattgaagcatttatcagggttattgtctcatgagcggatacatatttgaatgtatttagaaaaataa |
| | | acaaataggggttccgcgcacatttccccgaaaagtgccacctgacgtctaagaaaccattattatcatg |
| | | acattaacctataaaaataggcgtatcacgaggccattcgtctcgcgcgtttcggtgatgacggtgaaaa |
| | | cctctgacacatgcagctcccggagacggtcacagcttgtctgtaagcggatgccgggagcagacaag |
| | | cccgtcagggcgcgtcagcgggtgttggcgggtgtcggggctggcttaactatgcggcatcagagca |
| | | gattgtactgagagtgcaccatatgcggtgtgaaataccgcacagatgcgtaaggagaaaataccgcat |
| | | caggcgccattcgccattcaggctgcgcaactgttgggaagggcgatcggtgcgggcctcttcgctatt |
| | | acgccagctggcgaaagggggatgtgctgcaaggcgattaagttgggtaacgccagggttttcccagt |
| | | cacgacgttgtaaaacgacggccagtgccaagctg |
| 15 | MSLN amino | MALPTARPLLGSCGTPALGSLLFLLFSLGWVQPSRTLAGETGQEA |
| | acid | APLDGVLANPPNISSLSPRQLLGFPCAEVSGLSTERVRELAVALAQ |
| | sequence: | KNVKLSTEQLRCLAHRLSEPPEDLDALPLDLLLFLNPDAFSGPQA |
| | human | CTRFFSRITKANVDLLPRGAPERQRLLPAALACWGVRGSLLSEAD |
| | mesothelin | VRALGGLACDLPGRFVAESAEVLLPRLVSCPGPLDQDQQEAARA |
| | sequence | ALQGGGPPYGPPSTWSVSTMDALRGLLPVLGQPIIRSIPQGIVAA |
| | (UniProt | WRQRSSRDPSWRQPERTILRPRFRREVEKTACPSGKKAREIDESLI |
| | Accession | FYKKWELEACVDAALLATQMDRVNAIPFTYEQLDVLKHKLDEL |
| | No. | YPQGYPESVIQHLGYLFLKMSPEDIRKWNVTSLETLKALLEVNKG |
| | Q13421) | HEMSPQVATLIDRFVKGRGQLDKDTLDTLTAFYPGYLCSLSPEEL |
| | | SSVPPSSIWAVRPQDLDTCDPRQLDVLYPKARLAFQNMNGSEYF |
| | | VKIQSFLGGAPTEDLKALSQQNVSMDLATFMKLRTDAVLPLTVA |
| | | EVQKLLGPHVEGLKAEERHRPVRDWILRQRQDDLDTLGLGLQG |
| | | GIPNGYLVLDLSMQEALSGTPCLLGPGPVLTVLALLLASTLA |
| 53 | Single | EVQLVESGGGLVQPGGSLRLSCAASGGDWSANFMYWYRQAPGK |
| | domain | QRELVARISGRGVVDYVESVKGRFTISRDNSKNTLYLQMNSLRA |
| | anti-MSLN | EDTAVYYCAVASYWGQGTLVTVSS |
| | binder 1 | |
| | (SD1) | |
| | (CDRs | |
| | under- | |
| | lined) | |
| 54 | Single | EVQLVESGGGLVQPGGSLRLSCAASGSTSSINTMYWYRQAPGKE |
| | domain | RELVAFISSGGSTNVRDSVKGRFTISRDNSKNTLYLQMNSLRAED |
| | anti-MSLN | TAVYYCNTYIPYGGTLHDFWGQGTLVTVSS |
| | binder 4 | |
| | (SD4) | |
| | (CDRs | |
| | under- | |
| | lined) | |
| 55 | Single | QVQLVESGGGVVQAGGSLRLSCAASGSTFSIRAMRWYRQAPGTE |
| | domain | RDLVAVIYGSSTYYADAVKGRFTISRDNSKNTLYLQMNSLRAED |
| | anti-MSLN | TAVYYCNADTIGTARDYWGQGTLVTVSS |
| | binder 6 | |
| | (SD6) | |
| | (CDRs | |
| | under- | |
| | lined) | |

What is claimed is:

1. A method for the treatment of a mesothelin (MSLN)-expressing unresectable, metastatic, or recurrent cancer in a human subject in need thereof, the method comprising administering to the human subject a first dose comprising an amount of transduced anti-MSLN T cell receptor fusion protein (TFP) T cells, and optionally administering one or more additional doses of transduced anti-MSLN TFP T cells, wherein the first dose and each of the one or more additional doses comprise from about 5×10⁷ to about 1×10⁹ transduced cells/m².

2. The method of claim 1, wherein the human subject has been diagnosed with the cancer.

3. The method of claim 1, wherein the cancer is malignant pleural mesothelioma (MPM).

4. The method of claim 1, wherein the cancer is non-small cell lung cancer (NSCLC).

5. The method of claim 1, wherein the cancer is serous ovarian adenocarcinoma.

6. The method of claim 1, wherein the cancer is cholangiocarcinoma.

7. The method of claim 1, wherein the method comprises administering the one or more additional doses of anti-MSLN TFP T cells.

8. The method of claim 7, wherein the one or more additional doses of anti-MSLN TFP T cells comprise one, two, three, or more additional doses of anti-MSLN TFP T cells.

9. The method of claim 7, wherein the one or more additional doses comprise one, two, three, or more additional doses of anti-MSLN TFP T cells administered in evenly spaced increments.

10. The method of claim 7, wherein the one or more additional doses of anti-MSLN TFP T cells comprise a second dose, a third dose, and a fourth dose of anti-MSLN TFP T cells administered in evenly spaced increments.

11. The method of claim 1, wherein the anti-MSLN TFP T cells are administered via intravenous infusion.

12. The method of claim 1, wherein the anti-MSLN TFP T cells are administered as a single agent.

13. The method of claim 1, wherein each dose of anti-MSLN TFP T cells is between about 4.25×10⁷/m² to about 5.75×10⁷/m².

14. The method of claim 1, wherein the first dose of anti-MSLN TFP T cells is about 4.25×10⁷/m².

15. The method of claim 1, wherein the first dose of anti-MSLN TFP T cells is about 5×10⁷/m².

16. The method of claim 1, wherein the first dose of anti-MSLN TFP T cells is about 5.75×10⁷/m².

17. The method of claim 1, wherein the first dose of anti-MSLN TFP T cells is about 7.5×10⁷/m².

18. The method of claim 1, wherein the first dose of anti-MSLN TFP T cells is about 1×10⁸/m².

19. The method of claim 1, wherein the first dose of anti-MSLN TFP T cells is about 2.5×10⁸/m².

20. The method of claim 1, wherein the first dose of anti-MSLN TFP T cells is about 5×10⁸/m².

21. The method of claim 1, wherein the first dose of anti-MSLN TFP T cells is about 7.5×10⁸/m².

22. The method of claim 1, wherein the first dose of anti-MSLN TFP T cells is about 1×10⁹/m².

23. The method of claim 1, wherein a dose range of ±15% of a target dose is administered.

24. The method of claim 7, wherein a second dose of anti-MSLN TFP T cells is administered no sooner than 60 days following administration of the first dose of anti-MSLN TFP T cells and no later than 12 months following administration of the first dose of anti-MSLN TFP T cells.

25. The method of claim 1, wherein the method further comprises administering to the human subject a lymphodepleting chemotherapy regimen prior to administration of the first dose of anti-MSLN TFP T cells.

26. The method of claim 25, wherein the lymphodepleting chemotherapy regimen comprises administration of about four doses of fludarabine and about three doses of cyclophosphamide.

27. The method of claim 25, wherein the lymphodepleting chemotherapy regimen comprises administration of four doses of fludarabine and three doses of cyclophosphamide.

28. The method of claim 25, wherein the lymphodepleting chemotherapy regimen comprises administration of fludarabine at a level of about 30 mg/m²/day on days −7 through −4 relative to administration of anti-MSLN TFP T cells, and further comprises administration of cyclophosphamide at a level of about 600 mg/m²/day on days −6 through −4 relative to administration of anti-MSLN TFP T cells

29. The method of claim 25, wherein the lymphodepleting chemotherapy regimen comprises administration of fludarabine at a level of 30 mg/m²/day on days −7 through −4 relative to administration of anti-MSLN TFP T cells, and further comprises administration of cyclophosphamide at a level of 600 mg/m²/day on days −6 through −4 relative to administration of anti-MSLN TFP T cells.

30. The method of claim 1, wherein the method further comprises administering a chemotherapeutic agent to the human subject.

31. The method of claim 30, wherein the chemotherapeutic agent is administered four times at three dose levels, comprising a first dose, a second dose, a third dose, and a fourth dose.

32. The method of claim 31, wherein the first dose of the chemotherapeutic agent is administered three weeks after administration of anti-MSLN TFP T cells, and wherein subsequent doses are administered every three weeks thereafter.

33. The method of claim 30, wherein the chemotherapeutic agent is administered every three weeks.

34. The method of claim 30, wherein the chemotherapeutic agent comprises chemotherapy.

35. The method of claim 30, wherein the chemotherapeutic agent comprises pembrolizumab.

36. A method for the treatment of a mesothelin (MSLN)-expressing unresectable, metastatic, or recurrent cancer in a human subject in need thereof, the method comprising:
(a) administering to the human subject a lymphodepleting chemotherapy regimen;
(b) administering to the human subject a plurality of doses, each dose of the plurality of doses comprising an amount of transduced anti-MSLN TFP T cells T cells, at an interval of less than about sixty days between doses; and
(c) optionally administering to the human subject an effective amount of a chemotherapeutic agent.

37. The method of claim 36, wherein the cancer is malignant pleural mesothelioma (MPM).

38. The method of claim 36, wherein the cancer is non-small cell lung cancer (NSCLC).

39. The method of claim 36, wherein the cancer is serous ovarian adenocarcinoma.

40. The method of claim 36, wherein the cancer is cholangiocarcinoma.

41. The method of claim 36, comprising wherein the plurality of doses comprises a first dose, a second dose, a third dose, and a fourth dose of anti-MSLN TFP T cells.

42. The method of claim 41, wherein the plurality of doses of anti-MSLN TFP T cells are administered in evenly spaced increments.

43. The method of claim 42, wherein each dose of the plurality of doses comprises from about 5×10⁷ to about 1×10⁹ transduced cells/m².

44. The method of claim 42, wherein each dose of anti-MSLN TFP T cells is between about 4.25×10⁷/m² to about 5.75×10⁷/m².

45. The method of claim 43, wherein the first dose of anti-MSLN TFP T cells is about 4.25×10⁷/m².

46. The method of claim 43, wherein each dose of the plurality of doses is about 5×10⁷/m².

47. The method of claim 36, wherein each dose of the plurality of doses is about 7.5×10⁷/m².

48. The method of claim 43, wherein each dose of the plurality of doses is about 1×10⁸/m².

49. The method of claim 36, wherein each dose of the plurality of doses is about 2.5×10⁸/m².

50. The method of claim 43, wherein each dose of the plurality of doses is about 5×10⁸/m².

51. The method of claim 36, wherein each dose of the plurality of doses is about 7.5×10⁸/m².

52. The method of claim 43, wherein each dose of the plurality of doses is about 1×10⁹/m².

53. The method of claim 43, wherein a dose range of ±15% of a target dose of each dose of the plurality of doses is administered.

54. The method of claim 43, wherein a second dose of the plurality of doses of anti-MSLN TFP T cells is administered no sooner than 60 days following administration of a first dose of the plurality of doses and no later than 12 months following administration of the first dose of the plurality of doses of anti-MSLN TFP T cells.

55. The method of claim 36, wherein each dose of the plurality of doses is administered via intravenous infusion.

56. The method of claim 36, wherein the method further comprises administering to the human subject a lymphodepleting chemotherapy regimen prior to administration of a first dose of the plurality of doses of anti-MSLN TFP T cells.

57. The method of claim 56, wherein the lymphodepleting chemotherapy regimen comprises administration of four doses of fludarabine and three doses of cyclophosphamide.

58. The method of claim 56, wherein the lymphodepleting chemotherapy regimen comprises administration of fludarabine at a level of 30 mg/m²/day on days −7 through −4 relative to administration of anti-MSLN TFP T cells, and further comprises administration of cyclophosphamide at a level of 600 mg/m²/day on days −6 through −4 relative to administration of anti-MSLN TFP T cells.

59. The method of claim 56, wherein the method further comprises administering a chemotherapeutic agent to the human subject.

60. The method of claim 56, wherein the chemotherapeutic agent is administered four times at three dose levels, comprising a first dose, a second dose, a third dose, and a fourth dose.

61. The method of claim 60, wherein the first dose of the chemotherapeutic agent is administered three weeks after administration of anti-MSLN TFP T cells, and wherein subsequent doses are administered every three weeks thereafter.

62. The method of claim 56, wherein the chemotherapeutic agent is administered every three weeks.

63. The method of claim 56, wherein the chemotherapeutic agent comprises chemotherapy.

64. The method of claim 56, wherein the chemotherapeutic agent comprises pembrolizumab.
